(19) United States
(12) Patent Application Publication
(10) Pub. No.: US 2024/0050545 A1
(43) Pub. Date: Feb. 15, 2024

(54) NOVEL TRYPANOSOMAL VACCINE

(71) Applicant: GENOME RESEARCH LIMITED, Hinxton (GB)

(72) Inventors: Gavin Wright, Hinxton (GB); Delphine Autheman, Hinxton (GB)

(21) Appl. No.: 18/249,030

(22) PCT Filed: Oct. 14, 2021
(86) PCT No.: PCT/GB2021/052666

§ 371 Date: Apr. 13, 2023

(30) Foreign Application Priority Data
Oct. 14, 2020 (GB) 2016270.7

(51) Int. Cl. A61K 39/005; A61P 33/02; A61P 37/04

(52) U.S. Cl. CPC A61K 39/005; A61P 33/02; A61P 37/04; A61K 2039/55577

(57) ABSTRACT

The present invention relates to a trypanosomal vaccine comprising an FLA1 binding protein, as well as to pharmaceutical compositions comprising said vaccine and their uses in vaccination to prevent or treat trypanosomal infection in a mammal. Thus, also provided are a method of preventing or treating trypanosomal infection comprising administering said vaccine and a kit of parts comprising a medical instrument or other means for administering.

### CROSS-REFERENCE TO RELATED APPLICATIONS

This application is a U.S. National Stage Application under 35 U.S.C. § 371 which claims the benefit of priority to International Patent Application No. PCT/GB2021/052666, filed Oct. 14, 2021, which claims the benefit of priority to GB Patent Application No. 2016270.7 filed Oct. 14, 2020, each of which is hereby incorporated by reference in its entirety.

### SEQUENCE LISTING

The application contains a Sequence Listing that has been filed electronically in the form of a text file, created Oct. 26, 2023, and named “WEL-C-P2899PCT_Corrected_Sequence_Listing_ST25.txt” (365 kilobytes), the contents of which are incorporated herein by reference in their entirety.

### FIELD OF THE INVENTION

The invention relates to a trypanosomal vaccine, to pharmaceutical compositions comprising said vaccine and to their uses in vaccination to prevent or treat trypanosomal infection in a mammal.

### BACKGROUND OF THE INVENTION

The livelihoods of millions of people living in Africa are at risk due to infectious diseases that affect the health of livestock animals that provide them with essential food, milk, clothing and draught power. One major livestock disease is animal African trypanosomiasis (AAT) which is caused by blood-dwelling Trypanosome parasites that affect many important farm animals including cattle, goats, sheep, horses, and pigs. AAT is endemic from the Southern edge of the Sahara to Zimbabwe/Mozambique and is estimated to cause annual productivity losses of over $1 billion, representing a major barrier for the socioeconomic advancement of many African countries. Such is the impact of this disease that the United Nations Food and Agricultural Organisation consider it to “lie at the heart of Africa's struggle against poverty”.

The disease is mainly caused by two species of trypanosome: T. congolense and T. vivax which are transmitted through the bite of an infected tsetse fly. The few drugs available for AAT are not satisfactory: they cause serious side effects, and parasite resistance to these drugs is increasing. Importantly, even if new effective drugs were developed, these trypanosome parasites are endemic in wild animals meaning there would be little chance of eradicating the disease, and so livestock animals would require constant monitoring and treatment. The best solution would be the deployment of an effective vaccine; however, vaccinating against trypanosome infections has long been considered unachievable because the surface of these parasites is immunologically protected by a highly abundant cell surface protein called the variable surface glycoprotein (VSG). VSGs comprise a large family of related but not identical proteins, and trypanosomes express a small number or even a single variant on their surface at any one time. Host antibodies to VSG alleles are able to kill parasites; however, individual parasites within a population of trypanosomes can switch between variants and those that have switched to an antigenically distinct variant are able to effectively evade the host immune response ensuring the survival of the population as a whole.

One commonly-used strategy in the development of vaccines is to use inactivated or attenuated parasites, however, these vaccines are difficult to manufacture and can sometimes cause outbreaks if not appropriately attenuated. Modern vaccines, therefore, are typically purified recombinant proteins that can elicit protective immune responses and are consequently chemically defined.

Leishmania is a related genus of trypanosomes which are responsible for the disease leishmaniasis. They are spread by sandflies of the genus Phlebotomus in the Old World, and of the genus Lutzomyia in the New World. At least 93 sandfly species are proven or probable vectors worldwide. Their primary hosts are vertebrates; Leishmania commonly infects hyraxes, canids, rodents, and humans.

There is therefore a great need to provide an alternative and effective vaccine against trypanosomes such as the Trypanosoma and Leishmania species.

### SUMMARY OF THE INVENTION

According to a first aspect of the invention, there is provided a trypanosomal vaccine comprising an FLA1 binding protein.

According to a further aspect of the invention, there is provided a pharmaceutical composition comprising the trypanosomal vaccine as defined herein.

According to a further aspect of the invention, there is provided a method of preventing or treating trypanosomal infection in a mammal which comprises administering to the mammal a therapeutically effective amount of the vaccine composition as defined herein.

According to a further aspect of the invention, there is provided a method of inducing an immune response in a mammal, wherein the method includes administering to the mammal, an effective amount of the vaccine composition as defined herein.

According to a further aspect of the invention, there is provided a kit of parts comprising a vaccine composition as defined herein, a medical instrument or other means for administering the vaccine composition and instructions for use.

### BRIEF DESCRIPTION OF THE FIGURES

FIG. 1: Expression and purification of the extracellular regions of TcIL3000_0_35140 and TcIL3000_0_17090. Proteins consisting of the entire ectodomains of TcIL3000_0_35140 and TcIL3000_0_17090 were expressed as a soluble recombinant protein in HEK293 cells and purified from spent tissue culture media using immobilised metal ion chromatography. Approximately one microgram of each purified protein was resolved by SDS-PAGE under reducing conditions. The protein migrated as a series of glycoforms around the expected mass.

FIG. 2: Vaccination with the ectodomains of TcIL3000_0_35140 confers protection in a murine model of T. congolense infection. (A) Five animals were vaccinated with TcIL3000_0_35140 (solid line, filled diamonds) show attenuated T. congolense parasitaemia relative to adjuvant-only control animals (dotted lines, open circles). Parasitaemia was quantified in each animal by bioluminescence using the firefly luciferase gene transgenically expressed by the T. congolense strain used and plotted as a function of time post-infection. Survival curves indicate when animals were withdrawn from the study. (B) Comparisons of the parasitaemia on the indicated days post-infection in the vaccinated and control animals. Data points represent individual animals and horizontal bar represents mean±s.d. Comparisons were made using an one-way ANOVA with Dunnett's multiple comparison test for statistical confidence where *P≤0.01; **P≤0.001; ****P≤0.00001. (C) Exemplar bioluminescence images of three control animals (numbers 1 to 3) and three vaccinated (numbers 4 to 6) on the indicated days post-infection. A cross indicates that the animal was removed from the study.

FIG. 3: Repeat vaccinations with an independent preparation of TcIL3000_0_35140 in a larger cohort conforms vaccine effect in a murine model of T. congolense infection. (A) Fifteen animals were vaccinated with TcIL3000_0_35140 (solid line filled diamonds) show attenuated T. congolense parasitaemia relative to adjuvant-only control animals (dotted lines open circles). Parasitaemia was quantified in each animal by bioluminescence using the firefly luciferase gene transgenically expressed by the T. congelense strain used and plotted as a function of time post-infection. Survival curves indicate when animals were withdrawn from the study. (B) Comparisons of the parasitaemia on the indicated days post-infection in the vaccinated and control animals. Data points represent individual animals and horizontal bar represents mean±s.d. Comparisons were made using the student t-test where statistical confidence is indicated as ****P≤0.00001.

FIG. 4: Vaccination with the ectodomains of TcIL3000_0_17090 confers protection in a murine model of T. congolense infection. (A) Five animals were vaccinated with TcIL3000_0_17090 (solid line, filled diamonds) show attenuated T. congolense parasitaemia relative to adjuvant-only control animals (dotted lines, open circles). Parasitaemia was quantified in each animal by bioluminescence using the firefly luciferase gene transgenically expressed by the T. congolense strain used and plotted as a function of time post-infection. Survival curves indicate when animals were withdrawn from the study. (B) Comparisons of the parasitaemia on the indicated days post-infection in the vaccinated and control animals. Data points represent individual animals and horizontal bar represents mean±s.d. Comparisons were made using a one-way ANOVA with Dunnett's multiple comparison test for statistical confidence where *P≤0.01; **P≤0.001; ****P≤0.00001. (C) Exemplar bioluminescence images of three control animals (numbers 1 to 3) and three vaccinated (numbers 4 to 6) on the indicated days post-infection. A cross indicates that the animal was removed from the study.

FIG. 5: Passive transfer of immunity to Trypanosoma congolense infections with anti-TcIL3000_0_17090 immune sera. Mice received three doses of 100 or 200 microlitres of immune sera from animals immunised with the ectodomain of TcIL3000_0_17090 or control sera and challenged with a bioluminescent T. congolense parasite. Mice dosed with immune sera showed a dose-dependent reduction in parasitaemia compared to those receiving control sera. Bars indicate mean t SD, groups were compared by one-way ANOVA with Sidak post-hoc test *P≤0.01, ****P≤0.00001.

FIG. 6: Mice vaccinated with recombinant TcIL3000_0_35140 representing the “Savannah” strain of T. congolense are able to cross-protect against challenge with a “Forest-type” strain. (A) Nine mice were immunized with purified soluble TcIL3000_0_35140 recombinant protein adjuvanted in Quil-A and challenged with “Forest-type” T. congolense strain called DIN80 (solid line, filled diamonds). Parasitaemia was quantified on the indicated days after parasite challenge by microscopy; controls are a cohort of eight animals treated with adjuvant only (dotted line, open circles). Vaccinated animals were partially protected from the infection compared to the control animals with one animal showing no evidence of parasitaemia up until day 22. (B) Statistical comparison of the data shown in (A) which compares the parasitaemia in the TcIL3000_0_35140-vaccinated animals (filled diamonds) to controls (open circles) on the indicated days. Bars indicate mean t SD, groups were compared by an unpaired t test; ns=not significant, ***P≤0.0001, ****P≤0.00001. Data points represent individual animals and grey shading indicates the limits of detection.

FIG. 7: Mice vaccinated with both recombinant IFX and TcIL3000_0_17090 are able to control infections from both T. vivax and T. congolense. (A) T. vivax challenge. Mice were vaccinated with purified soluble IFX (dot-dash line, filled circles) or a combination of both IFX and TcIL3000_0_17090 in a co-administration protocol using Quil-A as an adjuvant (solid line, filled squares) and challenged with a transgenic luciferase-expressing T. vivax line. Vaccinated animals from both groups were partially protected from the infection compared to the control animals (dotted line, unfilled circles) with seven out of ten IFX-alone vaccinated animals, and five out of ten IFX-TcIL3000_0_17090 vaccinated animals showing evidence of sterile protection. (B) T. congolense challenge. Vaccinated mice were challenged with a bioluminescent T. congolense parasite with those animals vaccinated with IFX-alone (dot-dash line, filled circles) showing no evidence of protection as expected. Animals vaccinated with either TcIL3000_0_17090 alone (dashed line, unfilled diamonds) or both IFX and TcIL3000_0_17090 (solid line, filled squares) showed evidence of protection against T. congolense infection compared to controls (dotted line, cross-filled diamonds). Data points represent individual animals and grey shading indicates the limits of detection.

### DETAILED DESCRIPTION OF THE INVENTION

According to a first aspect of the invention, there is provided a trypanosomal vaccine comprising an FLA1 binding protein.

References herein to FLA1 binding protein refer to the flagellum adhesion protein 1 (FLA1), a glycosylated, transmembrane protein essential for flagellum attachment and cell division.

The present invention relates to the identification of non-variant cell surface T. congolense proteins, which, when used in the context of a vaccine can elicit protective immune responses. Using the genome sequence to identify potential candidates, a pair of related vaccine target antigens have been identified which, when produced as a purified recombinant protein and administered with an appropriate immunostimulatory adjuvant, confers protection to T. congolense infections in mice. The key finding of the invention is recognition that these two candidate vaccines are both FLA1 binding proteins. The results presented herein indicate that these non-variant parasite proteins will be an important component of a vaccine to prevent AAT in livestock animals. This finding has applicability to vaccines in other species which contain orthologs of FLA11 binding proteins, such as Leishmania.

In one embodiment, the FLA binding protein comprises the amino acid sequence as set forth in SEQ ID NO: 1, or a protein having at least 90% sequence identity to said amino acid sequence, or a fragment of said amino acid sequence thereof, or a nucleic acid molecule encoding said protein.

References herein to the amino acid sequence set forth in SEQ ID NO: 1 refer to:

(SEQ ID NO: 1)
AVAHVKRNRRVETVAGAYGLTGMVDGVPPDSRLSSPMAICRGRTADEILV
GTASGLRTYSRSSGELGTLFTSSVKVVGGSTGGSAYGNPRSCVHRGIDNS
SVIYFVDGQKDVKYYKNNEVLSKDTVANASLTAMTIFGGSLYMTDQINKA
LVTCKLSADGAPHDCLSKKKLNDTCGENTFTGITSTAKGIFIAGQGSTTP
GNICWIGLDDTTVTKLGQGEYVDVSSTSSGDLYAVSKTQIFHLEPAGSAP
QLKTVAGVKGTPCLPTPDGEDIRFCELNKILAIADHELYVTSERSHLLRA
VILPPVRVQAVFSGRPVPVGYPEGDTLDWIVENLVKDVNEALQTTESLID
PSTVYVDPDTWTTRFVALVQQSDFDDAATERALGEGNYTYITAALDEYYN
ETDQAVYMDSVMVPYCSEAALDAIRRRIAEEARRVLDFPLIYADMPVELE
GSGVENVTMVKLLMPASFNNETVSELLEAADLTGFAHSAIKEMRGGETRV
SVVLPNPPFNFSGVTPDVDQDIRWYVHGNVMKQLDICEKLNAKGAAPAPE
PVEDGNESGGGVVYTGEFCQSSITNRTETQNLKPPYDQKNTYEIFLPNKY
DFNASWCVDIVDWRELNDWLSNVTAGSHIEDASWCGQGCII.

The amino acid sequence of SEQ ID NO: 1 corresponds to the ectodomain of a cell surface T. congolense protein known as TcIL3000_0_17090.

The full length amino acid sequence of TcIL3000_0_17090 is shown below:

(SEQ ID NO: 2)
MRTGRALQVLLHATIISLGLVECAVAHVKRNRRVETVAGAYGLTGMVDGV
PPDSRLSSPMAICRGRTADEILVGTASGLRTYSRSSGELGTLFTSSVKVV
GGSTGGSAYGNPRSCVHRGIDNSSVIYFVDGQKDVKYYKNNEVLSKDTVA
NASLTAMTIFGGSLYMTDQINKALVTCKLSADGAPHDCLSKKKLNDTCGE
NTFTGITSTAKGIFIAGQGSTTPGNICWIGLDDTTVTKLGQGEYVDVSST
SSGDLYAVSKTQIFHLEPAGSAPQLKTVAGVKGTPCLPTPDGEDIRFCEL
NKILAIADHELYVTSERSHLLRAVILPPVRVQAVESGRPVPVGYPEGDTL
DWIVENLVKDVNEALQTTESLIDPSTVYVDPDTWTTRFVALVQQSDEDDA
ATERALGEGNYTYITAALDEYYNETDQAVYMDSVMVPYCSEAALDAIRRR
IAEEARRVLDFPLIYADMPVELEGSGVENVTMVKLLMPASFNNETVSELL
EAADLTGFAHSAIKEMRGGETRVSVVLPNPPFNFSGVTPDVDQDIRWYVH
GNVMKQLDICEKLNAKGAAPAPEPVEDGNESGGGVVYTGEFCQSSITNRT
ETQNLKPPYDQKNTYEIFLPNKYDFNASWCVDIVDWRELNDWLSNVTAGS
HIEDASWCGQGCIIALAVVGALLTTGLVVVAVVLTSKRRRLAAVVAPPRP
KFVSATEDEED.

The underlined portion represents the ectodomain region of TcIL3000_0_17090.

Data is presented herein which surprisingly shows that vaccinating animals with a recombinant protein comprising the entire ectodomain of TcIL3000_0_17090 T. congolense cell surface protein confers protection in a mouse model of infection demonstrating that this protein could be an effective subunit vaccine and therefore represents a very attractive candidate for preventing or treating T. congolense infection.

In an alternative embodiment, the FLA1 binding protein comprises the amino acid sequence as set forth in SEQ ID NO: 3, or a protein having at least 90% sequence identity to said amino acid sequence, or a fragment of said amino acid sequence thereof, or a nucleic acid molecule encoding said protein.

References herein to the amino acid sequence set forth in SEQ ID NO: 3 refer to:

(SEQ ID NO: 3)
HVKRNRRVETVAGAYGLTGMVDGVPPDSRLSSPMAICRGRTADEILVGTA
SGLRTYSRSSGELGTLFTSSVKVVGGSTGGSAYGNPRSCVHRGIDNSSVI
YFVDGQKDVKYYKNNEVLSKDTVANASLTAMTIFGGSLYMTDQINKALVT
CKLSADGAPHDCLSKKKLNDTCGFNTFTGITSTAKGIFIAGQGSTTPGNI
CWIGLDDTTVTKLGQGEYVDVSSTSSGDLYAVSKTQIFHLEPAGSAPQLK
TVAGVKGTPCLPTPDGEDIRFCELNKILAIADHELYVTSERSHLLRAVIL
PPVRVQAVESGRPVPVGYPEGDTLDWIVENLVKDVNEALQTTESLIDPST
VYVDPDTWTTRFVALVQQSDFDDAATERALGEGNYTYITAALDEYYNETD
QAVYMDSVMVPYCSEAALDAIRRKIAEEARRVLDFPLIYADMPVELEGSG
AENVTMVKLLMPASFNNETVSELLEAADLTGFAHSAIKEMRGGETRVSVV
LPNPPFNFSGVTPDVDQDIRWYVHGNVMKQLDICEKLNAKGAAPAPEPVE
DSNESGGGVVYTGEFCQSSITNRTETQNLKPPYDQKNTYEIFLPNKYDFN
ASWCVDIVDWRELNDWLSNVTVGSHIEDASWCGQGCI.

The amino acid sequence of SEQ ID NO: 3 corresponds to the ectodomain of a cell surface T. congolense protein known as TcIL3000_0_35140.

The full length amino acid sequence of TcIL3000_0_35140 is shown below:

(SEQ ID NO: 4)
MRTGRALQLLLHATIIFLGLVECAVAHVKRNRRVETVAGAYGLTGMVDGVP
PDSRLSSPMAICRGRTADEILVGTASGLRTYSRSSGELGTLFTSSVKVVGG
STGGSAYGNPRSCVHRGIDNSSVIYFVDGQKDVKYYKNNEVLSKDTVANAS
LTAMTIFGGSLYMTDQINKALVTCKLSADGAPHDCLSKKKLNDTCGENTFT
GITSTAKGIFIAGQGSTTPGNICWIGLDDTTVTKLGQGEYVDVSSTSSGDL
YAVSKTQIFHLEPAGSAPQLKTVAGVKGTPCLPTPDGEDIRFCELNKILAI
ADHELYVTSERSHLLRAVILPPVRVQAVESGRPVPVGYPEGDTLDWIVENL
VKDVNEALQTTESLIDPSTVYVDPDTWTTRFVALVQQSDFDDAATERALGE
GNYTYITAALDEYYNETDQAVYMDSVMVPYCSEAALDAIRRKIAEEARRVL
DFPLIYADMPVELEGSGAENVTMVKLLMPASFNNETVSELLEAADLTGFAH
SAIKEMRGGETRVSVVLPNPPFNFSGVTPDVDQDIRWYVHGNVMKQLDICE
KLNAKGAAPAPEPVEDSNESGGGVVYTGEFCQSSITNRTETQNLKPPYDQK
NTYEIFLPNKYDFNASWCVDIVDWRELNDWLSNVTVGSHIEDASWCGQGCI
IALAVVGALLTTGLVVVAVVLTSKRRRLAAVVAPPRPKFVSATEDEED.

The underlined portion represents the ectodomain region of TcIL3000_0_35140.

Data is presented herein which surprisingly shows that vaccinating animals with a recombinant protein comprising the entire ectodomain of TcIL3000_0_35140 T. congolense cell surface protein confers protection in a mouse model of infection demonstrating that this protein could be an effective subunit vaccine and therefore represents a very attractive candidate for preventing or treating T. congolense infection.

It will be appreciated that references herein to “identity” are to be understood as meaning the percentage identity between two protein sequences, e.g.: SEQ ID NO: X and SEQ ID NO: 1 or SEQ ID NO: X and SEQ ID NO: 3, which is the sum of the common amino acids between aligned sequences SEQ ID NO: X and SEQ ID NO: 1 or SEQ ID NO: X and SEQ ID NO: 3, divided by the shorter length of either SEQ ID NO: X or SEQ ID NOs: 1 or 3, expressed as a percentage.

In one embodiment, the protein of the invention has greater than 90% sequence identity with the ectodomain region of TcIL3000_0_17090 (SEQ ID NO: 1), such as at least 91%, 92%, 93%, 94%, 95%, 96%, 97%, 98% or 99% sequence identity with the ectodomain region of TcIL3000_0_17090 (SEQ ID NO: 1).

In an alternative embodiment, the protein of the invention has greater than 90% sequence identity with the ectodomain region of TcIL3000_0_35140 (SEQ ID NO: 3), such as at least 91%, 92%, 93%, 94%, 95%, 96%, 97%, 98% or 99% sequence identity with the ectodomain region of TcIL3000_0_35140 (SEQ ID NO: 3).

References herein to ‘fragment’ include, for example, functional fragments with a C-terminal truncation, or with an N-terminal truncation. Fragments are suitably greater than 10 amino acids in length, for example greater than 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500 amino acids in length.

In a further embodiment, the protein of the invention consists of the amino acid sequence as set forth in SEQ ID NO: 1.

In an alternative embodiment, the protein of the invention consists of the amino acid sequence as set forth in SEQ ID NO: 3.

In an alternative embodiment, the vaccine comprises a nucleic acid molecule encoding said protein of the invention. References herein to “nucleic acid molecule” typically refers to DNA or RNA. In a further embodiment, the nucleic acid molecule comprises an oligonucleotide encoding said protein.

References herein to “trypanosomal” refer to a genus of kinetoplastids (class Kinetoplastida), a monophyletic group of unicellular parasitic flagellate protozoa. The name is derived from the Greek trypano-(borer) and soma (body) because of their corkscrew-like motion. Most trypanosomes are heteroxenous (requiring more than one obligatory host to complete life cycle) and most are transmitted via a vector. The majority of species are transmitted by blood-feeding invertebrates, but there are different mechanisms among the varying species. Some, such as Trypanosoma equiperdum, are spread by direct contact. In an invertebrate host they are generally found in the intestine, but normally occupy the bloodstream or an intracellular environment in the mammalian host.

It will be appreciated that references herein to trypanosomal include both Trypanosoma species and Leishmania species of bacteria.

Examples of Trypanosoma species include: T. ambystomae, T. antiquus, T. avium, T. boissoni, T. brucei, T. brucei gambiense, T. brucei rhodesiense, T. cruzi, T. congolense, T. equinum, T. equiperdum, T. evansi, T. everetti, T. hosei, T. irwini, T. lewisi, T. melophagium, T. paddae, T. parroti, T. percae, T. rangeli, T. rotatorium, T. rugosae, T. sergenti, T. simiae, T. sinipercae, T. suis, T. theileri, T. triglae, T. tungarae and T. vivax.

In one embodiment, the trypanosomal vaccine is a T. congolense, T. brucei, T. brucei gambiense, T. brucei rhodesiense, T. cruzi or T. evansi, vaccine. In a further embodiment, the trypanosomal vaccine is a T. congolense vaccine. Examples of FLA1 binding proteins from T. brucei, T. brucei gambiense, T. cruzi or T. evansi include the following:

Trypanosoma brucei

| | |
|---|---|
| Tb427.05.4570 | >Tb427.05.4570\|Trypanosoma brucei Lister strain 427\|hypothetical |
| | protein, conserved\|protein\|length = 818 |
| | MCFIFGVEMSNLAKRPMSLRKLPQLFLLIMIGIAFVAVECIGTPVKLP |
| | RRVDTVAGQFGVEGETNGYPNTTRLTEPYALCRGRTNDEILVGSSNS |
| | FRNYSRKTKETGXYLRYNVGDSVISGSSTINKPRSCVRRGSGNHTIIY |
| | FVDDQKDIKYIVGDDVSSFSVPTXGSLNAVAVHEGSLYVTDQNNKS |
| | VWKCGLGGAGKPQSCEEKKFTSVTLDAKPEGIAVTSKGIFVTARDSS |
| | NKGALLWLDMNGSNRKGNVSGGFVDVFSTESGMLYAATEKELYTV |
| | TATDTSLSVTLFAGKNTSSCYFPTNGEDIVLCDNSRLLVIEEYEMYVT |
| | SRAKHTMRALTLPPVNLTAIFRGRPAPVGYPNTTIMEQFVASLTEDV |
| | NKALGTNDSYVDPDSVRVDPDTWETNXTVFVQQTRFDNTTEEKLRS |
| | LTYTQTDKTVDEYYGLTDEYVYIDTVLVPFCDDASLVTIQRALAREA |
| | GRALNFSLVYADKPITFGSDVAENVTAVKLLMPHSFKNATTPKQLSA |
| | ANLTDFAHNLVKDLRASDTRVDITFPDPPFNFSAVVPEREQEVRWFV |
| | HGKVMKQLEICERLGSQGDAAVIAAAADATARGKANVTLNTSGVK |
| | ANDTGVGPNTTNTAGGANTTANVATNGTANVIVNPSTNATPTGTSN |
| | ASVTNTTERAVPVVAPTQPSNGYAECRSAITNRTETQNMEPPYDRKH |
| | RYEVFLPKKYDFNVSWCVDIIDWRDLDEMLNNRTDEVVEKSLSWCG |
| | HGCIIAFAVVGSLIAACLVVLAVVLTSKRRRLAAVVAPPRPKFVSTVE |
| | DDEEDRVSNIGVPLTDGKGTTAP (SEQ ID NO: 5) |
| Tb427.05.4580 | >Tb427.05.4580\|Trypanosoma brucei Lister strain 427\|hypothetical |
| | protein, conserved\|protein\|length = 818 |
| | MCFIFGVEMSNLAKRPMSLRKLPQLFLLIMIGIAFVAVECIGAPVKLP |
| | RRVDTVAGQFGFDGTTDGSSNVSMLSSPYALCRGRTNDEILVGSSNS |
| | FRNYSRKTKETGTFLRGGPTGGLVSADAKISKPRSCVRRGSGNHTIIY |
| | FVDDQNGLKYINDNEIQHVTVGDGLSLTSVAIYEKDLYVTDQNNKS |
| | VWRCNVGGAGKPQNCEEKKFTGLTFTAKPEGIAVTSKGIFVAARDSS |
| | NKGALLWLDMNGGGSKGNVSGGFVDVESTESGMLYAATEKELYTV |
| | TATDSXFSVTLFAGKNTSSCYSHANGEDIVLCDNSRLLVIEEYEMYV |
| | TSXEKHTMRALTLPPVNLTAIFRGRPAPVGYPNTTIMEQFVASLTEDV |
| | NKALGTNDSYVDPDSVRVDPDTWETNXTVFVQQTRFDNTTEEKLRS |
| | LTYTQTDKTVDEYYGLTDEYVYIDTVLVPFCDDASLVTIQRALAREA |
| | GRALNFSLIYADKPITFGSDVAENVTAVKLLMPHSFKNATTPKQLSA |
| | ANLTDFAHNLVKDLRASDTRVDITFPDPPFNFSAVVPEREQEVRWFV |
| | HGKVMKQLEICERLGSQGDAAVIAAAAAATARGKANVTLNTSGVK |
| | ANDTGVGPNTTNTAGGANTTANVVANGTANVIVNPSTNATPTGTSN |
| | ASVTNTTERAVPVVAPTQPSNGYAECRSAITNRTETQNMEPPYDRKH |
| | RYEVFLPKKYDFNVSWCVDIIDWRDLDEMLNNRTDEVVEKSLSWCG |
| | HGCIIAFAVVGSLIAACLVVLAVVLTSKRRRLAAVVAPPRPKFVSTVE |
| | DDEEDRVSNIGVPLTDGKGTTAP (SEQ ID NO: 6) |
| Tb427.08.4050 | >Tb427.08.4050\|Trypanosoma brucei Lister strain 427\|hypothetical |
| | protein, conserved\|protein\|length = 750 |
| | MPLWKQTNCEVETMNVREVVGTVHLGYVSQMLLLVATVXTIVVRS |
| | GAAPIELKRHVTTVAGKYGHIGDKDGFPGMSELSSPHAMCRGRNSD |
| | EILLGTVDRFRAFSRKNRETTTITAWETDEDQXSGSRSVKVDKPRAC |
| | VQWTVGGSTFVYFVESMGEVKYFKDSGVFSHDVVRNGSLTGVALY |
| | GNHLYLTEQNTNTVWTCEVGSDGDPIACHSHVALSAXCSIYGPIGIA |
| | ATQQGIFVVARGPAKQGTICWFDLQGHKIAEVDGEYVDITSTRSGDL |
| | LAATQNELHRVSTDGNKLTTKRFAGGSTNSCLPNTEGDDTLLCEITR |
| | LLVVTEYEMYVTSEKKSVLRSVTLPPVYVQGXFPGRPLPVGYPDKDI |
| | MEWIVGNLTEDINTALGTTESIVASSSVHVDSTTWLTNFTAGVQQPD |
| | FDDEKTEQALHKSNYEHTKEAADEYYNLTDEQVYMDSTMVPYCNR |
| | LSLDALRRKLAKEAGEVLNFTLIYADMPLKAESSDAGNITTVKLLMP |
| | ASFNNTVTHDLLSDANLTETAHSFIKYLRSSDTHVDVTFSNPPFNESS |
| | LTPDEEQEVRWYIHDEVMNQIKKCEERSTGRSMARREEVGDYSRTTI |
| | ATALDSNVTGVCQSTITNRTVSLFYQPPYVEMSLYEVFIPGNYTFDVS |
| | ECVGEIDWQDLNDHLNNDTVRPTTEKAPKCGRVCLIIIAVVCALIVA |
| | VLIVLAVVFTSKRRRLAAVVAPARPKFVSTLDEDEQDYASAYGNKE |
| | RVEQ (SEQ ID NO: 7) |
| Tb427.08.4100 | >Tb427.08.4100\|Trypanosoma brucei Lister strain 427\|hypothetical |
| | protein, conserved\|protein\|length = 750 |
| | MPLWKQTNCEVETMNVREVVGTVHLGYVSQMLLLVATVXTIVVRS |
| | GAAPIELKRHVTTVAGKYGHIGDKDGFPGMSELSSPHAMCRGRNSD |
| | EILLGTVDRFRAFSRKNRETTTITAWETDEDQXSGSRSVKVDKPRAC |
| | VQWTVGGSTFVYFVESMGEVKYFKDSGVFSHDVVRNGSLTGVALY |
| | GNHLYLTEQNTNTVWTCEVGSDGDPIACHSHVALSAXCSIYGPIGIA |
| | ATQQGIFVVARGPAKQGTICWFDLQGHKIAEVDGEYVDITSTRSGDL |
| | LAATQNELHRVSTDGNKLTTKRFAGGSTNSCLPNTEGDDTLLCEITR |
| | LLVVTEYEMYVTSEKKSVLRSVTLPPVYVQGXFPGRPLPVGYPDKDI |
| | MEWIVGNLTEDINTALGTTESIVASSSVHVDSTTWLTNFTAGVQQPD |
| | FDDEKTEQALHKSNYEHTKEAADEYYNLTDEQVYMDSTMVPYCNR |
| | LSLDALRRKLAKEAGEVLNFTLIYADMPLKAESSDAGNITTVKLLMP |
| | ASFNNTVTHDLLSDANLTETAHSFIKYLRSSDTHVDVTFSNPPFNESS |
| | LTPDEEQEVRWYIHDEVMNQIKKCEERSTGRSMARREEVGDYSRTTI |
| | ATALDSNVTGVCQSTITNRTVSLFYQPPYVEMSLYEVFIPGNYTFDVS |
| | ECVGEIDWQDLNDHLNNDTVRPTTEKAPKCGRVCLIIIAVVCALIVA |
| | VLIVLAVVFTSKRRRLAAVVAPARPKFVSTLDEDEQDYASAYGNKE |
| | RVEQ (SEQ ID NO: 8) |
| Tb427_050053100 | >Tb427_050053100\|Trypanosoma brucei Lister strain 427 2018\| |
| | hypothetical protein, conserved\|protein\|length = 818 |
| | MCFIFGVEMSNLAKRPMSLRKLPQLFLLIMIGIAFVAVECIGAPVKLP |
| | RRVDTVAGQFGVEGETNGYPNTTRLTEPYALCRGRTNDEILVGSSNS |
| | FRNYSRKTKETGTYLRYNVGDSVISGSSTINKPRSCVRRGSGNHTIIYF |
| | VDDQKDIKYIVGDDVSSFSVPTRGSLNAVAVHEGSLYVTDQNNKSV |
| | WKCGLGGAGKPQSCEEKKFTSVTLDAKPEGIAVTSKGIFVAARDSSN |
| | KGALLWLDMNGSNRKGNVSGGFVDVESTESGMLYAATEKELYTVT |
| | ATDSAFSVTLFAGKNTSQCYFPTNGEDIVLCDNSRLLVIEEYEMYVTS |
| | KEKHTMRALTLPPVNLTAIFRGRPAPVGYPNTTIMEQFVASLTEDVN |
| | KALGTNDSYVDPDSVRVDPDTWETNFTVFVQQTRFDNTTEEKLRSL |
| | TYTQTDKTVDEYYGLTDEYVYIDTVLVPFCDDASLVTIQRALAREAG |
| | RALNFSLIYADKPITFGSDVAENVTAVKLLMPHSFKNATTPKQLSAA |
| | NLTDFAHNLVKDLRASDTRVDITFPDPPFNFSAVVPEREQEVRWFVH |
| | GKVMKQLEICERLGSQGDAAVIAAAAAATARGKANVTLNTSGVKA |
| | NDTGVGPNTTNTAGGANTTANVVANGTANVIVNPSTNATPTGTSNA |
| | SVTNTTERAVPVVAPTQPSNGYAECRSAITNRTETQNMEPPYDRKHR |
| | YEVFLPKKYDFNVSWCVDIIDWRDLDEMLNNRTDEVVEKSLSWCG |
| | HGCIIAFAVVGSLIAACLVVLAVVLTSKRRRLAAVVAPPRPKFVSTVE |
| | DDEEDRVSNIGVPLTDGKGTTAP (SEQ ID NO: 9) |
| Tb427_050053300 | >Tb427_050053300\|Trypanosoma brucei Lister strain 427 2018\| |
| | hypothetical protein, conserved\|protein\|length = 818 |
| | MCFIFGVEMSNLAKRPMSLRKLPQLFLLIMIGIAFVAVECIGAPVKLP |
| | RRVDTVAGQFGVEGETNGYPNTTRLTEPYALCRGRTNDEILVGSSNS |
| | FRNYSRKTKETGTFLRGGPTGGLVSADAKISKPRSCVRRGSGNHTIIY |
| | FVDDQNGLKYINDNEIQHVTVGDGLSLTSVAIYEKDLYVTDQNNKS |
| | VWRCNVGGAGKPQNCEEKKFTGLTFTAKPEGIAVTSKGIFVAARDSS |
| | NKGALLWLDMNGSNRKGNVSGGFVDVFSTESGMLYAATEKELYTV |
| | TATDSAFSVTLFAGKNTSSCYSHANGEDIVLCDNSRLLVIEEYEMYV |
| | TSMEKHTMRALTLPPVNLTAIFRGRPAPVGYPNTTIMEQFVASLTED |
| | VNKALGTNDSYVDPDSVRVDPDTWETNFTVFVQQTRFDNTTEEKLR |
| | SLTYTQTDKTVDEYYGLTDEYVYIDTVLVPFCDDASLVTIQRALARE |
| | AGRALNFSLIYADKPITFGSDVAENVTAVKLLMPHSFKNATTPKQLS |
| | AANLTDFAHNLVKDLRASDTRVDITFPDPPFNFSAVVPEREQEVRWF |
| | VHGKVMKQLEICERLGSQGDAAVIAAAAAVTARGKANVTLNTSGV |
| | KANDTGVGPNTTNTAGGANTTANVVANGTANVIVNPSTNATPTGTS |
| | NASATNTTERAVPVVAPTQPSNGYAECRSAITNRTETQNMEPPYDRK |
| | HRYEVFLPKKYDFNVSWCVDIIDWRDLDEMLNNRTDEVVEKSLSWC |
| | GHGCIIAFAVVGSLIAACLVVLAVVLTSKRRRLAAVVAPPRPKFVST |
| | VEDDEEDRVSNIGVPLTDGKGTTAP (SEQ ID NO: 10) |
| Tb427_050053500 | >Tb427_050053500\|Trypanosoma brucei Lister strain 427 2018\| |
| | hypothetical protein, conserved\|protein\|length = 810 |
| | MCFIFGVEMSNLAKRPMSLRKLPQLFLLIMIGIAFVAVECIGAPVKLP |
| | RRVDTVAGQFGVEGETNGYPNTTRLTEPYALCRGRTNDEILVGSSNS |
| | FRNYSRKTKETGTFLRGGPTGGLVSADAKISKPRSCVRRGSGNHTIIY |
| | FVDDQNGLKYINDNEIQHVTVGDGLSLTSVAIYEKDLYVTDQNNKS |
| | VWRCNVGGAGKPQNCEEKKFTGLTFTAKPEGIAVTSKGIFVAARDSS |
| | NKGALLWLDMNGSNRKGNVSGGFVDVFSTESGMLYAATEKELYTV |
| | TATDSAFSVTLFAGKNTSSCYSHANGEDIVLCDNSRLLVIEEYEMYV |
| | TSKEKHTMRALTLPPVNLTAIFRGRPAPVGYPNTTIMEQFVASLTEDV |
| | NKALGTNDSYVDPDSVRVDPDTWETNYTVFVQQTRFDNTTEEKLRS |
| | LTYTQTDKTVDEYYGLTDEYVYIDTVLVPFCDDASLVTIQRALAREA |
| | GRALNFSLIYADKPITFGSDVAENVTAVKLLMPHSFKNATTPKQLSA |
| | ANLTDFAHNLVKDLRASDTRVDITFPDPPFNFSAVVPEREQEVRWFV |
| | HGKVMKQLEICERLGSQGDAAVIAAAADATARGKANVTLNTSGVK |
| | ANDTGVGPNTTNTAGGANTTANVATNGTANVIVNPSTNASVTNTTE |
| | RAVPVVAPTQPSNGYAECRSAITNRTETQNMEPPYDRKHRYEVFLPK |
| | KYDFNVSWCVDIIDWRDLDEMLNNRTDEVVEKSLSWCGHGCIIAFA |
| | VVGSLIAACLVVLAVVLTSKRRRLAAVVAPPRPKFVSTVEDDEEDRV |
| | SNIGVPLTDGKGTTAP (SEQ ID NO: 11) |
| Tb427_080045300 | >Tb427_080045300\|Trypanosoma brucei Lister strain 427 2018\| |
| | hypothetical protein, conserved\|protein\|length = 750 |
| | MPLWKQTNCEVETMNVREVVGTVHLGYVSQMLLLVATVATIVVRS |
| | GAAPIELKRHVTTVAGKYGHIGDKDGFPGMSELSSPHAMCRGRNSD |
| | EILLGTVDRFRAFSRKNRETTTITAWETDEDQKSGSRSVKVDKPRAC |
| | VQWTVGGSTFVYFVESMGEVKYFKDSGVFSHDVVRNGSLTGVALY |
| | GNHLYLTEQNTNTVWTCEVGSDGDPIACHSHVALSANCSIYGPIGIA |
| | ATQQGIFVVARGPAKQGTICWFDLQGHKIAEVDGEYVDITSTRSGDL |
| | LAATQNELHRVSTDGNKLTTKRFAGGSTNSCLPNTEGDDTLLCEITR |
| | LLVVTEYEMYVTSEKKSVLRSVTLPPVYVQGLFPGRPLPVGYPDKDI |
| | MEWIVGNLTEDINTALGTTESIVASSSVHVDSTTWLTNFTAGVQQPD |
| | FDDEKTEQALHKSNYEHTKEAADEYYNLTDEQVYMDSTMVPYCNR |
| | LSLDALRRKLAKEAGEVLNFTLIYADMPLKAESSDAENITTVKLLMP |
| | ASFNNTVTHDLLSDANLTETAHSFIKYLRSSDTHVDVTFSNPPFNFSS |
| | LTPDEEQEVRWYIHDEVMNQIKKCEERSTGRSMARREEVGDHSRTTI |
| | ATALDSNVTGVCQSTITNRTVSLFYQPPYVEMSLYEVFIPGNYTEDVS |
| | ECVGEIDWQDLNDHLNNDTVRPTTEKAPKCGRVCLIIIAVVCALIVA |
| | VLIVLAVVFTSKRRRLAAVVAPARPKFVSTLDEDEQDYASAYGNKE |
| | RVEQ (SEQ ID NO: 12) |
| Tb427_080045800 | >Tb427_080045800\|Trypanosoma brucei Lister strain 427 2018\| |
| | hypothetical protein, conserved\|protein\|length = 750 |
| | MPLWKQTNCEVETMNVREVVGTVHLGYVSQMLLLVATVETIVVRS |
| | GAAPIELKRHVTTVAGKYGHIGDKDGFPGMSELSSPHAMCRGRNSD |
| | EILLGTVDRFRAFSRKNRETTTITAWETDEDQNSGSRSVKVDKPRAC |
| | VQWTVGDSTFVYFVESMGEVKYFKDSGVFSHDVVRNGSLTGVALY |
| | GNHLYLTEQNTNTVWTCEVGSDGDPIACHSHVALSAKCSIYGPIGIA |
| | ATQQGIFVVARGPAKQGTICWFDLQGHKIAEVDGEYVDITSTRSGDL |
| | LAATQNELHRVSTDGNKLTTKRFAGGSTNSCLPNTEGDDTLLCEITR |
| | LLVVTEYEMYVTSEKKSVLRSVTLPPVYVQGVFPGRPLPVGYPDKDI |
| | MEWIVGNLTEDINTALGTTESIVASSSVHVDSTTWLTNFTAGVQQPD |
| | FDDEKTEQALHKSNYEHTKEAADEYYNLTDEQVYMDSTMVPYCNR |
| | LSLDALRRKLAKEAGEVLNFTLIYADMPLKAESSDAGNITTVKLLMP |
| | ASFNNTVTHDLLSDANLTETAHSFIKYLRSSDTHVDVTFSNPPFNESS |
| | LTPDEEQEVRWYIHDEVMNQIKKCEERSTGRSMARREEVGDYSRTTI |
| | ATALDSNVTGVCQSTITNRTVSLFYQPPYVEMSLYEVFIPGNYTFDVS |
| | ECVGEIDWQDLNDHLNNDTVRPTTEKAPKCGRVCLIIIAVVCALIVA |
| | VLIVLAVVFTSKRRRLAAVVAPARPKFVSTLDEDEQDYASAYGNKE |
| | RVEQ (SEQ ID NO: 13) |
| Tb927.5.4570 | >Tb927.5.4570\|Trypanosoma brucei brucei TREU927\|Flagellum adhesion |
| | protein 3\|protein\|length = 818 |
| | MCFIFGVEMSNLAKRPMSLRKLPQLLLLIMIGIAFVAVECIGAPVKLP |
| | RRVDTVAGQFGVEGETNGYPNTTRLTEPYALCRGRINDEILVGSSNS |
| | FRNYSRKTKETGTYLRYNVGDSVISGSSTINKPRSCVRRGSGNHTIIYF |
| | VDDQKDIKYIVGDDVSSFSVPTSGSLNAVAVHEGTLYVTDQNNKSV |
| | WKCGLGGAGKPQSCEEKKFTSVTLDAKPEGIAVTSKGIFVTARDSSN |
| | KGALLWLDMSGGNRKGNVSGGFVDVESTESGVLYAATEKELYTVT |
| | ATDTSLSVTSFAGKNTSQCYFPTNGEDIVLCDNSRLLVIEEYEMYVTS |
| | KAKHTMRALTLPPVNLTAIFRGRPAPVGYPNTTIMEQFVASLTEDVN |
| | KALGTNDSYVDPDSVRVDPDTWETNFTVFVQQTRFDNTTEEKLRSL |
| | TYTQTDKTVDEYYGLTDEYVYIDTVLVPFCDDASLVTIQRALAREAG |
| | RALNFSLVYADKPITFGSDVAENVTAVKLLMPHSFKNATTPKQLSAA |
| | NLTDFAHNLVKDLRASDTRVDITFPDPPFNFSAVVPEREQEVRWFVH |
| | GKVMKQLEICERLGSQGDAAVIAAAADATARGKANVTLNTSGVKA |
| | NDTGVGPNTTNTAGGANTTANVAANGTANVIVNPSTNATPTGTTNA |
| | SVTNTTERAVPVVAPTQPSNGYAECRSAITNRTETQNMEPPYDRKHR |
| | YEVFLPKKYDFNVSWCVDIIDWRDLDEMLNNRTDEVVEKSLSWCG |
| | HGCIIAFAVVGSLIAACLVVLAVVLTSKRRRLAAVVAPPRPKFVSTVE |
| | DDDEDRVSNIGVPLTDGKGTTAP (SEQ ID NO: 14) |
| Tb927.5.4580 | >Tb927.5.4580\|Trypanosoma brucei brucei TREU927\|Flagellum adhesion |
| | protein 3\|protein\|length = 818 |
| | MCFIFGVEMSNLAKRPMSLRKLPQLLLLIMIGIAFVAVECIGAPVKLP |
| | RRVDTVAGQFGFDGTTDGSSNVSMLSSPYALCRGRTNDEILVGSSNS |
| | FRNYSRKTKETGTFLRGGPTGGLVSADAKISKPRSCVRRGSGNHTIIY |
| | FVDDQNGLKYINDNEIQHVTVGNGLSLTSVAIYEKDLYVTDQNNKS |
| | VWRCNVGGAGKPQNCEEKKFTGLTFTAKPEGIAVTSKGIFVAARDSS |
| | NKGALLWLDMNGGGSKGNVSGGFVDVESTESGMLYAATEKELYTV |
| | TATGSAFSVTSFAGKNTSSCYSHANGEDIVLCDNSRLLVIEEYEMYV |
| | TSKEKHTMRALTLPPVNLTAIFRGRPAPVGYPNTTIMEQFVASLTEDV |
| | NKALGTNDSYVDPDSVRVDPDTWETNFTVFVQQTRFDNTTEEKLRS |
| | LTYTQTDKTVDEYYGLTDEYVYIDTVLVPFCDDASLVTIQRALAREA |
| | GRALNFSLIYADKPITFGSDVAENVTAVKLLMPHSFKNATTPKQLSA |
| | ANLTDFAHNLVKDLRASDTRVDITFPDPPFNFSAVVPEREQEVRWFV |
| | HGKVMKQLEICERLGSQGDAAVIAAAAAATARGKANVTLNTSGVK |
| | ANDTGVGPNTTNTAGGANTTANVVANGTANVIVNPSTNATPTGTTN |
| | ASVTNTTERAVPVVAPTQPSNGYAECRSAITNRTETQNMEPPYDRKH |
| | RYEVFLPKKYDFNVSWCVDIIDWRDLDEMLNNRTDEVVEKSLSWCG |
| | HGCIIAFAVVGSLIAACLVVLAVVLTSKRRRLAAVVAPPRPKFVSTVE |
| | DDEEDRVSNIGVPLTDGKGTTAP (SEQ ID NO: 15) |
| Tb927.8.4050 | >Tb927.8.4050\|Trypanosoma brucei brucei TREU927\|FLA1-binding |
| | protein\|protein\|length = 750 |
| | MPLWKQTNCEVETMNVREVVGTVHLGYVSQMLLLVATVATIVVRS |
| | GAAPIELKRHVTTVAGKYGHIGDKDGFPGMSELSSPHAMCRGRNSD |
| | EILLGTVDRFRAFSRKNRETTTITAWETDEDQKSGSRSVKVDKPRAC |
| | VQWTVGGSTFVYFVESMGEVKYFKDSGVFSHDVVRNGSLTGVALY |
| | GNHLYLTEQNTNTVWTCEVGSDGDPIACHSHVALSANCSIYGPIGIA |
| | ATQQGIFVVARGPAKQGTICWFDLQGHKIAEVDGEYVDITSTRSGDL |
| | LAATQNELHRVSTDGNKLTTKRFAGGSTNSCLPNTEGDDTLLCEITR |
| | LLVVTEYEMYVTSEKKSVLRSVTLPPVYVQGLFPGRPLPVGYPDKDI |
| | MEWIVGNLTEDINTALGTTESIVASSSVHVDSTTWLTNFTAGVQQPD |
| | FDDEKTEQALHESNYEHTKEAADEYYNLTDEQVYMDSTMVPYCNR |
| | LSLDALRRKLAKEAGEVLNFTLIYADMPLKAESSDAENITTVKLLMP |
| | ASFNNTVTHDLLSDANLTETAHSFIKYLRSSDTHVDVTFSNPPFNESS |
| | LTPDEEQEVRWYIHDEVMNQIKKCEERSTGRSMARREEVGDHSRTTI |
| | ATALDSNVTGVCQSTITNRTVSLFYQPPYVEMSLYEVFIPGNYTFDVS |
| | ECVGEIDWQDLNDHLNNDTVRPTTEKAPKCGRVCLIIIAVVCALIVA |
| | VLIVLAVVFTSKRRRLAAVVAPARPKFVSTLDEDEQDYASAYGNKE |
| | RVEQ (SEQ ID NO: 16) |
| Tb927.8.4100 | >Tb927.8.4100\|Trypanosoma brucei brucei TREU927\|FLA1-binding |
| | protein\|protein\|length = 750 |
| | MPLWKQTNCEVETMNVREVVGTVHLGYVSQMLLLVATVATIVVRS |
| | GAAPIELKRHVTTVAGKYGHIGDKDGFPGMSELSSPHAMCRGRNSD |
| | EILLGTVDRFRAFSRKNRETTTITAWETDEDQKSGSRSVKVDKPRAC |
| | VQWTVGGSTFVYFVESMGEVKYFKDSGVFSHDVVRNGSLTGVALY |
| | GNHLYLTEQNTNTVWTCEVGSDGDPIACHSHVALSANCSIYGPIGIA |
| | ATQQGIFVVARGPAKQGTICWFDLQGHKIAEVDGEYVDITSTRSGDL |
| | LAATQNELHRVSTDGNKLTTKRFAGGSTNSCLPNTEGDDTLLCEITR |
| | LLVVTEYEMYVTSEKKSVLRSVTLPPVYVQGLFPGRPLPVGYPDKDI |
| | MEWIVGNLTEDINTALGTTESIVASSSVHVDSTTWLTNFTAGVQQPD |
| | FDDEKTEQALHESNYEHTKEAADEYYNLTDEQVYMDSTMVPYCNR |
| | LSLDALRRKLAKEAGEVLNFTLIYADMPLKAESSDAENITTVKLLMP |
| | ASFNNTVTHDLLSDANLTETAHSFIKYLRSSDTHVDVTFSNPPFNESS |
| | LTPDEEQEVRWYIHDEVMNQIKKCEERSTGRSMARREEVGDHSRTTI |
| | ATALDSNVTGVCQSTITNRTVSLFYQPPYVEMSLYEVFIPGNYTFDVS |
| | ECVGEIDWQDLNDHLNNDTVRPTTEKAPKCGRVCLIIIAVVCALIVA |
| | VLIVLAVVFTSKRRRLAAVVAPARPKFVSTLDEDEQDYASAYGNKE |
| | RVEQ (SEQ ID NO: 17) |
| Tb05.5K5.210 | >Tb05.5K5.210\|Trypanosoma brucei brucei TREU927\|hypothetical |
| | protein\|protein\|length = 818 |
| | MCFIFGVEMSNLAKRPMSLRKLPQLLLLIMIGIAFVAVECIGAPVKLP |
| | RRVDTVAGQFGVEGETNGYPNTTRLTEPYALCRGRTNDEILVGSSNS |
| | FRNYSRKTKETGTYLRYNVGDSVISGSSTINKPRSCVRRGSGNHTIIYF |
| | VDDQKDIKYIVGDDVSSFSVPTSGSLNAVAVHEGSLYVTDQNNKSV |
| | WKCGLGGAGKPQSCEEKKFTSVTLDAKPEGIAVTSKGIFVTARDSSN |
| | KGALLWLDMSGSNRKGNVSGGFVDVESTESGVLYAATEKELYTVT |
| | ATDTSLSVTLFAGKNTSQCYFSTNGEDIVLCDNSRLLVIEEYEMYVTS |
| | KEKHTMRALTLPPVNLTAIFRGRPAPVGYPNTTIMEQFVASLTEDVN |
| | KALGTNDSYVDPDSVRVDPNTWETNYTVFVQQTRFDNTTEEKLRSL |
| | TYTQTDKTVDEYYGLTDEYVYIDTVLVPFCDDASLVTIQRALAREAG |
| | RALNFSLIYADKPITFGSDVAENVTAVKLLMPHSFKNATTPKQLSAA |
| | NLTDFAHNLVKDLRASDTRVDITFPDPPFNFSAVVPEREQEVRWFVH |
| | GKVMKQLEICERLGSQGDAAVIAAAADATARGKANVTLNTSGVKA |
| | NDTGVGPNTTNTAGGADTTANVAANGTANVIVNPSTNATPTGTTNA |
| | SVTNTTERAVPVVAPTQPSNGYAECRSAITNRTETQNMEPPYDRKHR |
| | YEVFLPKKYDFNVSWCVDIIDWRDLDEMLNNRTDEVVEKSLSWCG |
| | HGCIIAFAVVGSLIAACLVVLAVVLTSKRRRLAAVVAPPRPKFVSTVE |
| | DDEEDRVSNIGVPLTDGKGTTAP (SEQ ID NO: 18) |
| Tb05.5K5.220 | >Tb05.5K5.220\|Trypanosoma brucei brucei TREU927\|hypothetical |
| | protein\|protein\|length = 818 |
| | MCFIFGVEMSNLAKRPMSLRKLPQLLLLIMIGIAFVAVECIGAPVKLP |
| | RRVDTVAGQFGFDGTTDGSSNVSMLSSPYALCRGRTNDEILVGSSNS |
| | FRNYSRKTKETGTFLRGGPTGGLVSADAKISKPRSCVRRGSGNHTIIY |
| | FVDDQNGLKYINDNEIQHVTVGNGLSLTSVAIYEKDLYVTDQNNKS |
| | VWRCNVGGAGKPQNCEEKKFTGLTFTAKPEGIAVTSKGIFVAARDSS |
| | NKGALLWLDMNGGGSKGNVSGGFVDVESTESGMLYAATEKELYTV |
| | TATDSAFSVTSFAGKNTSSCYSHANGEDIVLCDNSRLLVIEEYEMYV |
| | TSKEKHTMRALTLPPVNLTAIFRGRPAPVGYPNTTIMEQFVASLTEDV |
| | NKALGTNDSYVDPDSVRVDPDTWETNFTVFVQQTRFDNTTEEKLRS |
| | LTYTQTDKTVDEYYGLTDEYVYIDTVLVPFCDDASLVTIQRALAREA |
| | GRALNFSLIYADKPITFGSDVAENVTAVKLLMPHSFKNATTPKQLSA |
| | ANLTDFAHNLVKDLRASDTRVDITFPDPPFNFSAVVPEREQEVRWFV |
| | HGKVMKQLEICERLGSQGDAAVIAAAAAATARGKANVTLNTSGVK |
| | ANDTGVGPNTTNTAGGANTTANVAANGTANVIVNPSTNATPTGTTN |
| | ASVTNTTERAVPVVAPTQPSNGYAECRSAITNRTETQNMEPPYDRKH |
| | RYEVFLPKKYDFNVSWCVDIIDWRDLDEMLNNRTDEVVEKSLSWCG |
| | HGCIIAFAVVGSLIAACLVVLAVVLTSKRRRLAAVVAPPRPKFVSTVE |
| | DDEEDRVSNIGVPLTDGKGTTAP (SEQ ID NO: 19) |
| Tb11.v5.0364 | >Tb11.v5.0364\|Trypanosoma brucei brucei TREU927\|hypothetical |
| | protein, conserved\|protein\|length = 820 |
| | LAMCFIFGVEMSNLAKRPMSLRKLPQLLLLIMIGIAFVAVECIGAPVK |
| | LPRRVDTVAGQFGFDGTTDGSSNVSMLSSPYALCRGRTNDEILVGSS |
| | NSFRNYSRKTKETGTFLRGGPTGGLVSADAKISKPRSCVRRGSGNHTI |
| | IYFVDDQNGLKYINDNEIQHVTVGNGLSLTSVAIYEKDLYVTDQNNK |
| | SVWRCNVGGAGKPQNCEEKKFTGLTFTAKPEGIAVTSKGIFVAARDS |
| | SNKGALLWLDMNGGGSKGNVSGGFVDVFSTESGMLYAATEKELYT |
| | VTATGSAFSVTSFAGKNTSSCYSHANGEDIVLCDNSRLLVIEEYEMY |
| | VTSKEKHTMRALTLPPVNLTAIFRGRPAPVGYPNTTIMEQFVASLTED |
| | VNKALGTNDSYVDPDSVRVDPDTWETNFTVFVQQTRFDNTTEEKLR |
| | SLTYTQTDKTVDEYYGLTDEYVYIDTVLVPFCDDASLVTIQRALARE |
| | AGRALNFSLIYADKPITFGSDVAENVTAVKLLMPHSFKNATTPKQLS |
| | AANLTDFAHNLVKDLRASDTRVDITFPDPPFNFSAVVPEREQEVRWF |
| | VHGKVMKQLEICERLGSQGDAAVIAAAADATARGKANVTLNTSGV |
| | KANDTGVGPNTTNTAGGANTTANVAANGTANVIVNPSTNATPTGTT |
| | NASVTNTTERAVPVVAPTQPSNGYAECRSAITNRTETQNMEPPYDRK |
| | HRYEVFLPKKYDFNVSWCVDIIDWRDLDEMLNNRTDEVVEKSLSWC |
| | GHGCIIAFAVVGSLIAACLVVLAVVLTSKRRRLAAVVAPPRPKFVST |
| | VEDDEEDRVSNIGVPLTDGKGTTAP (SEQ ID NO: 20) |

Trypanosoma brucei gambiense

| | |
|---|---|
| Tbg972.5.6160 | >Tbg972.5.6160\|Trypanosoma brucei gambiense |
| | DAL972\|hypothetical protein, conserved |
| | (fragment)\|protein\|length = 573 |
| | MSGSNRKGNVSGGFVDVFSTESGMLYAATEKELYTVTATDTSLSVT |
| | LFAGKNTSSCYSHANGEDIVLCDNSRLLVIEEYEMYVTSKEKHTMR |
| | ALTLPPVNLTAIFRGRPAPVGYPNTTIMEQFVASLTEDVNKALGTN |
| | DSYVDPDSVRVDPDTWETNYTVFVQQTRFDNTTEEKLRSLTYTQTD |
| | KTVDEYYGLTDEYVYIDTVLVPFCDDASLVTIQRALAREAGRALNF |
| | SLIYADKPITFGSDVAENVTAVKLLMPHSFKNATTPKQLSAANLTD |
| | FAHNLVKDLRASDTRVDITFPDPPFNFSAVVPEREQEVRWFVHGKV |
| | MKQLEICERLGSQGDAAVIAAAADATARGKANVTLNTSGVKANDTG |
| | VGPNTTNTAGGANTTANVATNGTANVIVNPSTNATPTGTSNASATN |
| | TTERAVPVVAPTQPSNGYAECRSAITNRTETQNMEPPYDRKHRYEV |
| | FLPKKYDFNVSWCVDIIDWRDLDEMLNNRTDEVVEKSLSWCGHGCI |
| | IAFAVVGSLIAACLVVLAVVLTSKRRRLAAVVAPPRPKFVSTVEDD |
| | EEDRVSNIGVPLTDGKGTTAP (SEQ ID NO: 21) |
| Tbg972.5.6180 | >Tbg972.5.6180\|Trypanosoma brucei gambiense |
| | DAL972\|hypothetical protein, conserved\| |
| | protein\|length = 818 |
| | MCFIFGVEMSNLAKRPMSLRKLPQLFLLIMIGIAFVAVECIGAPVK |
| | LPRRVDTVAGQFGFDGTTDGSSNVSMLSSPYALCRGRTNDEILVGS |
| | SNSFRNYSRKTKETGTFLRGGPTGGLVSADAKISKPRSCVRRGSGN |
| | HTIIYFVDDQNGLKYINDNEIQHVTVGNGLSLNAVAIHEKDLYVTD |
| | QNNKSVWRCNVGGAGKPQNCEEKKFTGVTFTAKPEGIAVTSKGIFV |
| | TARDSSNKGALLWLDMNGGGRKGNVSGGFVDVESTESGVLYAATEK |
| | ELYTVTATDTSLSVTLFAGKNTSQCYFPTNGEDIVLCDNSRLLVIE |
| | EYEMYVTSKAKHTMRALTLPPVNLTAIFRGRPAPVGYPNTTIMEQF |
| | VASLTEDVNKALGTNDSYVDPDSVRVDPDTWETNYTVFVQQTRFDN |
| | TTEEKLRSLTYTQTDKTVDEYYGLTDEYVYIDTVLVPFCDDASLVT |
| | IQRALAREAGRALNFSLIYADKPITFGSDVAENVTAVKLLMPHSFK |
| | NATTPKQLSAANLTDFAHNLVKDLRASDTRVDITFPDPPFNFSAVV |
| | PEREQEVRWFVHGKVMKQLEICERLGSQGDAAVIAAAADATARGKA |
| | NVTLNTSGVKANDTGVGPNTTNTAGGANTTANVATNGTANVIVNPS |
| | TNATPTGTSNASATNTTERAVPVVAPTQPSNGYAECRSAITNRTET |
| | QNMEPPYDRKHRYEVFLPKKYDFNVSWCVDIIDWRDLDEMLNNRTD |
| | EVVEKSLSWCGHGCIIAFAVVGSLIAACLVVLAVVLTSKRRRLAAV |
| | VAPPRPKFVSTVEDDEEDRVSNIGVPLTDGKGTTAP (SEQ ID |
| | NO: 22) |
| Tbg972.8.3750 | >Tbg972.8.3750\|Trypanosoma brucei gambiense |
| | DAL972\|FLA1-binding protein\|protein\|length = |
| | 750 |
| | MPLWKQTNCEVETMNVREVVGTVHLGYVSQMLLLVATVETIVVRSG |
| | AAPIELKRHVTTVAGKYGHIGDKDGFPGMSELSSPHAMCRGRNSDE |
| | ILLGTVDRFRAFSRKNRETTTITAWETDEDQNSGSRSVKVDKPRAC |
| | VQWTVGDSTFVYFVESMGEVKYFKDSGVFSHDVVRNGSLTGVALYG |
| | NHLYLTEQNTNTVWTCEVGSDGDPIACHSHVALSAKCSIYGPIGIA |
| | ATQQGIFVVARGPAKQGTICWFDLQGHKIAEVDGEYVDITSTRSGD |
| | LLAATQNELHRVSTDGNKLTTKRFAGGSTNSCLPNTEGDDTLLCEI |
| | TRLLVVTEYEMYVTSEKKSVLRSVTLPPVYVQGVFPGRPLPVGYPD |
| | KDIMEWIVGNLTEDINTALGTTESIVASSSVHVDSTTWLTNFTAGV |
| | QQPDFDDEKTEQALHKSNYEHTKEAADEYYNLTDEQVYMDSTMVPY |
| | CNRLSLDALRRKLAKEAGEVLNFTLIYADMPLKAESSDAGNITTVK |
| | LLMPASFNNTVTHDLLSDANLTETAHSFIKYLRSSDTHVDVTFSNP |
| | PFNFSSLTPDEEQEVRWYIHDEVMNQIKKCEERSTGRSMARREEVG |
| | DYSRTTIATALDSNVTGVCQSTITNRTVSLFYQPPYVEMSLYEVFI |
| | PGNYTFDVSECVGEIDWQDLNDHLNNDTVRPTTEKAPKCGRVCLII |
| | IAVVCALIVAVLIVLAVVFTSKRRRLAAVVAPARPKFVSTLDEDEQ |
| | DYASAYGNKERVEQ (SEQ ID NO: 23) |
| Tbg972.8.3800 | >Tbg972.8.3800\|Trypanosoma brucei gambiense |
| | DAL972\|FLA1-binding protein (fragment)\| |
| | protein\|length = 600 |
| | FVESIGEVKYFRDSGVFSHDVVRNGSLTGVALYGNHLYLTEQNTNT |
| | VWTCEVGSDGDPIACHSHVALSAKCSIYGPIGIAATQQGIFVVARG |
| | PAKQGTICWFDLQGHKIAEVDGEYVDITSTRSGDLLAATQNELHRV |
| | STDGNKLTTKRFAGGSTNSCLPNTEGDDTLLCEITRLLVVTEYEMY |
| | VTSEKKSVLRSVTLPPVYVQGVFPGRPLPVGYPDKDIMEWIVGNLT |
| | EDINTALGTTESIVASSSVHVDSTTWLTNFTAGVQQPDFDDEKTEQ |
| | ALHKSNYEHTKEAADEYYNLTDEQVYMDSTMVPYCNRLSLDALRRK |
| | LAKEAGEVLNFTLIYADMPLKAESSDAGNITTVKLLMPASFNNTVT |
| | HDLLSDANLTETAHSFIKYLRSSDTHVDVTFSNPPFNFSSLTPDEE |
| | QEVRWYIHDEVMNQIKKCEERSTGRSMARREEVGDYSRTTIATALD |
| | SNVTGVCQSTITNRTVSLFYQPPYVEMSLYEVFIPGNYTFDVSECV |
| | GEIDWQDLNDHLNNDTVRPTTEKAPKCGRVCLIIIAVVCALIVAVL |
| | IVLAVVFTSKRRRLAAVVAPARPKFVSTLDEDEQDYASAYGNKERV |
| | EQ (SEQ ID NO: 24) |

Trypanosoma cruzi

| TcBrA4_0018970 | >TcBrA4_0018970\|Trypanosoma cruzi Brazil A4\| |
|---|---|
| | hypothetical protein\|protein\|length = 151 |
| | MELPPPPLPIGYPDDNEVMKKIIQLMNEELNEHLKTNGTYVSQENMY |
| | VDANTWATKFAVMVQQHDFENATTPGEVLTTHFAQTKQFVKDYYDRV |
| | DEVLYMDTSIMPFCNDTMLNAVMHRLVSVVREVSVFRLFTPTRQRLG |
| | RNLILKTSPQ (SEQ ID NO: 25) |
| TcBrA4_0018980 | >TcBrA4_0018980\|Trypanosoma cruzi Brazil A4\| |
| | hypothetical protein, conserved\|protein\| |
| | length = 218 |
| | MDADMDAALLQILRELYGPENVVTLVFPMPEYDFSKLTDEQLVEIRW |
| | FILDMVRARLEECAVLSAGSVDASVSRHSGVCEAVITNRTETVISHP |
| | PFNIQSEYEVFVPSRYNFNASLCLDGIDWAVLEEVIKNYTEENKPRH |
| | KSACDRSCIIGLAVLAALVLTALIAVVVVLTSKRRRLAAVVAPVHPK |
| | FKSTLDEDEEEIETTNPLELKEEQRARDMY (SEQ ID NO: 26) |
| TcBrA4_0019750 | >TcBrA4_0019750\|Trypanosoma cruzi Brazil A4\| |
| | hypothetical protein, conserved\|protein\| |
| | length = 712 |
| | MFRFQSLLFALFTGLLFSFTSSVVVAMPLRYMVETVSGITGSIGHVN |
| | GGPGASLLTRPSAICQGRNEDELLFGTQGYFRNFSRSTKMTGILIGD |
| | GTAQILDGTWSQARIDGPRGCVRGIFNQKMIVYFVEGQSSLRYFTSN |
| | YVHTVIISINLSFTDVKLYEGKLYITEQTKDEVWVCDIDADGAPVSC |
| | ALKTGFKCDYGKYHGITVTKLGVFVVGESAAGICHFDMNGNKISVLG |
| | GNYIDVESLPSDELYIMSYTELLHLRVTGSAMAVEKFAGRADATCPP |
| | LIDGYDFTLCKNLRLFVIEQSEMYLATTLNTVRSVTLPPAIVWMELP |
| | PPPLPIGYPDDNEVMKKIIQLMNEELNEHLKTNGTYVSQENMYVDAN |
| | TWATKFAVMVQQHDFENATTPGEVLTTHFAQTKQFVKDYYDRVDEVL |
| | YMDTSIMPFCNDTMLNAVMHRLVSVVREVLGFPLIYANPPEARKEFN |
| | FENITTMKLLMPASFNNDTTRDALMDADMDAALLQILRELYGPENVV |
| | TLVFPMPEYDFSKLTDEQLVEIRWFILDMVRARLEECAVLSAGSVDA |
| | SVSRHSGVCEAVITNRTETVISHPPFNIQSEYEVFVPSRYNFNASLC |
| | LDGIDWAVLEEVIKNYTEENKPRHKSACDRSCIIGLAVLAALVLTAL |
| | IAVVVVLTSKRRRLAAVVAPVHPKFKSTLDEDEEEIETTNPLELKEE |
| | QRARDMY (SEQ ID NO: 27) |
| TcCLB.503571.19 | >TcCLB.503571.19\|Trypanosoma cruzi CL Brener |
| | Esmeraldo-like\|FLA1-binding protein\|protein\| |
| | length = 708 |
| | MSRFQRLLFALFAGFLFSFTASVVVAMPLRYMVETVSGITGSIGHVN |
| | GGPGTSLLTRPSAICQGRNEDELLFGTQGYFRNFSRSTKMTGILLGD |
| | GTVQILDGTWSQARIDGPRGCVRGIFNQKMIVYFVEGQSSLRYFTSN |
| | YVHTVTISINLSFTDVKLYEGKLYITEQTKDEVWGCDIDADGAPVSC |
| | ALKTGFKCDYGKYHGITVTKLGVFVVGESAAGICHFDMHGNKISVLG |
| | GNYIDVFSLPSDELYIMSYTELLHLRVIGSAMVVEKFAGRSDATCPP |
| | LIDGYDFTLCKNLRLFVIEQSEMYLATTLNTVRSVTLPPAIVWIELP |
| | PPPLPIGYPNDNEVMKKIIQLMNEELNKHLGTNGTYVSQETMHVDAN |
| | TWATKFAVMVQQQDFENATTPGEVLTTHFARTKQFVKDYYDRVNEVL |
| | YMDTSIMPFCNDTMLNAVMHRLVTVVREVLSFPLIYANPPEVRKEFD |
| | FENITTMKLLMPASFNNDTTREALMDADMDAALLQILRELYGPEHVV |
| | TLVFPMPQYDFSKLTDEQLVEVRWFILDLVRARLEECAVLSVDGVGA |
| | SVSSHSSVCEAVITNRTETVVSHPPFNIQSEYEVFVPSRYKFNASLC |
| | LDGIDWTVLEELIKNYTEENKPRHKSACDRSCIIGLAVLAALVLTAL |
| | IAVMVVLTSKRRRLAAVVAPVHPKFKSTLDEDEEEMETTNPLEVKDE |
| | QRA (SEQ ID NO: 28) |
| TcCLB.509561.9 | >TcCLB.509561.9\|Trypanosoma cruzi CL Brener |
| | Non-Esmeraldo-like\|FLA1-binding protein\| |
| | protein\|length = 366 |
| | MFWFQSLLFALFAGLLFSFTSSVVVAMPLRYMVETVSGITGSIGHAN |
| | GGPGTSLLTRPSAICQGRNEDELLFGTQGYFRNFSRSTKMTGILIGD |
| | GTAQILDGTWSQARIDGPRGCVRGIFSQKMIVYFVEGQSSLRYFTSD |
| | YVHTVMISINLFFTDVKLYEGKLYMTEQTKDEVWGCDIDADGAPVSC |
| | ALKTGFKCDYGKYHGITVTKQGVFVVGESAAGICHFDMHGNKLSVLG |
| | GNYIDVFSLPSDELYIMSYTELFHLRVIGSAMVVEKFAGRADATCPP |
| | LIDGYDFTLCKNLRLFVIDQSEMYLATTLNTVRSVTLPPAIVWMELP |
| | PPPLPIGYPDEYEVMKRIIQLMNEELNEHLRTNGTYV (SEQ ID |
| | NO: 29) |
| TCDM_03241 | >TCDM_03241\|Trypanosoma cruzi Dm28c 2014\| |
| | hypothetical protein\|protein\|length = 712 |
| | MFRFQSLLFALFTGLLFSFTSSVVVAMPLRYMVETVSGITGSIGHVN |
| | GGPGASLLTRPSAICKGRNEDELLFGTQGYFRNFSRSTKMTGILIGD |
| | GTAQILDGTWSQARIDGPRGCVRGIFNQKMIVYFVEGQSSLRYFTSN |
| | YVHTVIISINLSFTDVKLYEGKLYITEQTKDEVWVCDIDADGAPVSC |
| | ALKTGFKCDYGKYHGITVTKLGVFVVGESAAGICHFDMNGNKISVLG |
| | GNYIDVFSLPSDELYIMSYTELLHLRVTGSAMAVEKFAGRADATCPP |
| | LIDGYDFTLCKNLRLFVIEQSEMYLATTLNTVRSVTLPPAIVWMELP |
| | PPPLPIGYPDDNEVMKKIIQLMNEELNEHLKTNGTYVSQENMYVDAN |
| | TWATKFAVMVQQHDFENATTPGEVLTTHFAQTKQFVKDYYDRVDEVL |
| | YMDTSIMPFCNDTMLNAVMHRLVSVVREVLGFPLIYANPPEARKEFN |
| | FENITTMKLLMPASFNNDTTRDALMDADMDAALLQILWELYGPENVV |
| | TLVFPMPEYDFSKLTDEQLVEIRWFILDMVRARLEECAVLSAGSVDA |
| | SVSRHSGVCEAVITNRTETVISHPPFNIQSEYEVFVPSRYNFNASLC |
| | LDGIDWAVLEEVIKNYTEENKPRHKSACDRSCIIGLAVLAALVLTAL |
| | IAVVVVLTSKRRRLAAVVAPVHPKFKSTLDEDEEEIETTNPLELKEE |
| | QRARDMY (SEQ ID NO: 30) |
| BCY84_05332 | >BCY84_05332\|Trypanosoma cruzi Dm28c 2017\| |
| | hypothetical protein\|protein\|length = 712 |
| | MFRFQSLLFALFTGLLFSFTSSVVVAMPLRYMVETVSGITGSIGHVN |
| | GGPGASLLTRPSAICKGRNEDELLFGTQGYFRNFSRSTKMTGILIGD |
| | GTAQILDGTWSQARIDGPRGCVRGIFNQKMIVYFVEGQSSLRYFTSN |
| | YVHTVIISINLSFTDVKLYEGKLYITEQTKDEVWVCDIDADGAPVSC |
| | ALKTGFKCDYGKYHGITVTKLGVFVVGESAAGICHFDMNGNKISVLG |
| | GNYIDVFSLPSDELYIMSYTELLHLRVTGSAMAVEKFAGRADATCPP |
| | LIDGYDFTLCKNLRLFVIEQSEMYLATTLNTVRSVTLPPAIVWMELP |
| | PPPLPIGYPDDNEVMKKIIQLMNEELNEHLKTNGTYVSQENMYVDAN |
| | TWATKFAVMVQQHDFENATTPGEVLTTHFAQTKQFVKDYYDRVDEVL |
| | YMDTSIMPFCNDTMLNAVMHRLVSVVREVLGFPLIYANPPEARKEFN |
| | FENITTMKLLMPASFNNDTTRDALMDADMDAALLQILWELYGPENVV |
| | TLVFPMPEYDFSKLTDEQLVEIRWFILDMVRARLEECAVLSAGSVDA |
| | SVSRHSGVCEAVITNRTETVISHPPFNIQSEYEVFVPSRYNFNASLC |
| | LDGIDWAVLEEVIKNYTEENKPRHKSACDRSCIIGLAVLAALVLTAL |
| | IAVVVVLTSKRRRLAAVVAPVHPKFKSTLDEDEEEIETTNPLELKEE |
| | QRARDMY (SEQ ID NO: 31) |
| C4B63_21g106 | >C4B63_21g106\|Trypanosoma cruzi Dm28c 2018\| |
| | FLA1-binding protein\|protein\|length = 712 |
| | MFRFQSLLFALFTGLLFSFTSSVVVAMPLRYMVETVSGITGSIGHVN |
| | GGPGASLLTRPSAICKGRNEDELLFGTQGYFRNFSRSTKMTGILIGD |
| | GTAQILDGTWSQARIDGPRGCVRGIFNQKMIVYFVEGQSSLRYFTSN |
| | YVHTVIISINLSFTDVKLYEGKLYITEQTKDEVWVCDIDADGAPVSC |
| | ALKTGFKCDYGKYHGITVTKLGVFVVGESAAGICHFDMNGNKISVLG |
| | GNYIDVESLPSDELYIMSYTELLHLRVTGSAMAVEKFAGRADATCPP |
| | LIDGYDFTLCKNLRLFVIEQSEMYLATTLNTVRSVTLPPAIVWMELP |
| | PPPLPIGYPDDNEVMKKIIQLMNEELNEHLKTNGTYVSQENMYVDAN |
| | TWATKFAVMVQQHDFENATTPGEVLTTHFAQTKQFVKDYYDRVDEVL |
| | YMDTSIMPFCNDTMLNAVMHRLVSVVREVLGFPLIYANPPEARKEFN |
| | FENITTMKLLMPASFNNDTTRDALMDADMDAALLQILWELYGPENVV |
| | TLVFPMPEYDFSKLTDEQLVEIRWFILDMVRARLEECAVLSAGSVDA |
| | SVSRHSGVCEAVITNRTETVISHPPFNIQSEYEVFVPSRYNFNASLC |
| | LDGIDWAVLEEVIKNYTEENKPRHKSACDRSCIIGLAVLAALVLTAL |
| | IAVVVVLTSKRRRLAAVVAPVHPKFKSTLDEDEEEIETTNPLELKEE |
| | QRARDMY (SEQ ID NO: 32) |
| TcSYL_0095950 | >TcSYL_0095950\|Trypanosoma cruzi Sylvio X10/1\| |
| | unspecified product\|protein\|length = 217 |
| | MDADMDAALLQILRELYGPENVVTLVFPMPEYDFSKLTDEQLVEIRW |
| | FILDMVRARLEECAVLSAGSVDASVSRHSGVCEAVITNRTETVIPHP |
| | PFNIQSEYEVFVPSRYNFNASLCLDGIDWAVLEEVIKNYTEENKPRH |
| | KSACDRSCIIGLAVLAALVLTALIAVVVVLTSKRRRLAAVVAPVHPK |
| | FKSTLDEDEEEIETTNPLELKEEQRARDM (SEQ ID NO: 33) |
| TcSYL_0095960 | >TcSYL_0095960\|Trypanosoma cruzi Sylvio X10/1\| |
| | unspecified product\|protein\|length = 353 |
| | MIVYFVEGQSSLRYFTSNYVHTVIISINLSFTDVKLYEGKLYITEQT |
| | KDEVWVCDIDADGAPVSCALKTGFKCDYGKYHGITVTKLGVFVVGES |
| | AAGICHFDMNGNKISVLGGNYIDVFSLPSDELYIMSYTELLHLRVTG |
| | SAMAVEKFAGRADATCPPLIDGYDFTLCKNLRLFVIEQSEMYLATTL |
| | NTVRSVTLPPAIVWMELPPPPLPIGYPDDNEVMKKIIQLMNEELNEH |
| | LKTNGTYVSQENMYVDDNTWATKFAVMVQQHDFENATTPGEVLTTHF |
| | AQTKQFVKDYYDRVDEVLYMDTSIMPFCNDTMLNAVMHRLVSVVREV |
| | LVFRLFTPTRQRLGRNLILKTSPQ (SEQ ID NO: 34) |
| TCSYLVIO_000059 | >TCSYLVIO_000059\|Trypanosoma cruzi Sylvio |
| | X10/1-2012\| hypothetical protein\|protein\| |
| | length = 711 |
| | MFRFQSLLFALFTGLLFSFTSSVVVAMPLRYMVETVSGITGSIGHVN |
| | GGPGASLLTRPSAICQGRNEEELLFGTQGYFRNFSRSTKMTGILIGD |
| | GTAQILDGTWSQARIDGPRGCVRGIFNQKMIVYFVEGQSSLRYFTSN |
| | YVHTVIISINLSFTDVKLYEGKLYITEQTKDEVWVCDIDADGAPVSC |
| | ALKTGFKCDYGKYHGITVTKLGVFVVGESAAGICHFDMNGNKISVLG |
| | GNYIDVFSLPSDELYIMSYTELLHLRVTGSAMAVEKFAGRADATCPP |
| | LIDGYDFTLCKNLRLFVIEQSEMYLATTLNTVRSVTLPPAIVWMELP |
| | PPPLPIGYPDDNEVMKKIIQLMNEELNEHLKTNGTYVSQENMYVDAN |
| | TWATKFAVMVQQHDFENATTPGEVLTTHFAQTKQFVKDYYDRVDEVL |
| | YMDTSIMPFCNDTMLNAVMHRLVSVVREVLGFPLIYANPPEARKEFN |
| | FENITTMKLLMPASFNNDTTRDALMDADMDAALLQILRELYGPENVV |
| | TLVFPMPEYDFSKLTDEQLVEIRWFILDMVRARLEECAVLSAGSVDA |
| | SVSRHSGVCEAVITNRTETVIPHPPFNIQSEYEVFVPSRYNFNASLC |
| | LDGIDWAVLEEVIKNYTEENKPRHKSACDRSCIIGLAVLAALVLTAL |
| | IAVVVVLTSKRRRLAAVVAPVHPKFKSTLDEDEEEIETTNPLELKEE |
| | QRARDM (SEQ ID NO: 35) |
| C3747_125g76 | >C3747_125g76\|Trypanosoma cruzi TCC\|FLA1- |
| | binding protein\|protein\|length = 712 |
| | MSRFQRLLFALFAGFLFSFTASVVVAMPLRYMVETVSGITGSIGHVN |
| | GGPGTSLLTRPSAICQGRNEDELLFGTQGYFRNFSRSTKMTGILLGD |
| | GTVQILDGTWSQARIDGPRGCVRGIFNQKMIVYFVEGQSSLRYFTSN |
| | YVHTVTISINLSFTDVKLYEGKLYITEQTKDEVWGCDIDADGAPVSC |
| | ALKTGFKCDYGKYHGITVTKLGVFVVGESAAGICHFDMHGNKISVLG |
| | GNYIDVFSLPSDELYIMSYTELLHLRVIGSAMVVEKFAGRSDATCPP |
| | LIDGYDFTLCKNLRLFVIEQSEMYLATTLNTVRSVTLPPAIVWIELP |
| | PPPLPIGYPNDNEVMKKIIQLMNEELNKHLGTNGTYVSQETMHVDAN |
| | TWATKFAVMVQQQDFENATTPGEVLTTHFARTKQFVKDYYDRVNEVL |
| | YMDTSIMPFCNDTMLNAVMHRLVTVVREVLSFPLIYANPPEVRKEFD |
| | FENITTMKLLMPASFNNDTTREALMDADMDAALLQILRELYGPEHVV |
| | TLVFPMPQYDFSKLTDEQLVEVRWFILDLVRARLEECAVLSVDGVGA |
| | SVSSHSSVCEAVITNRTETVVSHPPFNIQSEYEVFVPSRYKFNASLC |
| | LDGIDWTVLEELIKNYTEENKPRHKSACDRSCIIGLAVLAALVLTAL |
| | IAVMVVLTSKRRRLAAVVAPVHPKFKSTLDEDEEEMETTNPLEVKDE |
| | QRARDMY (SEQ ID NO: 36) |
| C3747_66g107 | >C3747_66g107\|Trypanosoma cruzi TCC\|FLA1- |
| | binding protein\|protein length = 712 |
| | MFWFQSLLFALFAGLLFSFTSSVVVAMPLRYMVETVSGITGSIGHAN |
| | GGPGTSLLTRPSAICQGRNEDELLFGTQGYFRNFSRSTKMTGILIGD |
| | GTAQILDGTWSQARIDGPRGCVRGIFSQKMIVYFVEGQSSLRYFTSD |
| | YVHTVMISINLFFTDVKLYEGKLYMTEQTKDEVWGCDIDADGAPVSC |
| | ALKTGFKCDYGKYHGITVTKQGVFVVGESAAGICHFDMHGNKLSVLG |
| | GNYIDVFSLPSDELYIMSYTELFHLRVIGSAMVVEKFAGRADATCPP |
| | LIDGYDFTLCKNLRLFVIDQSEMYLATTLNTVRSVTLPPAIVWMELP |
| | PPPLPIGYPDDNEVMKRIIQLMNEELNEHLRTNGTYVSQENMHVDAD |
| | TWATKFAVMVQQHDFENATTPGEVLTTHFAQTTQFVKDYYDRVDEVL |
| | YMDTSIMPFCNDTMLNAVMHRLVSVVREVLGFPLIYANPPEARKELN |
| | FENITTMKLLMPASFNNDTTRDALMDADMDAALLQILRELYGPENVV |
| | TLVFPMPQYDFSKLTDEQLVEVRWFILDLVRARLEECDVLSASSVDA |
| | SVSSHSGVCEAVITNRTETVVSHPPFNVQSEYEVFVPLRYKFNASLC |
| | LDGIDWAVLEEIIKNYTEENKPRRKSACDRSCIIGLAVLAALVLTAL |
| | IAVVVVLTSKRRRLAAVVAPVHPKFKSTLDEDEEEIETTNPLEVKDE |
| | QRARDMY (SEQ ID NO: 37) |
| TcYC6_0063920 | >TcYC6_0063920\|Trypanosoma cruzi Y C6\| |
| | hypothetical protein, conserved\|protein\| |
| | length = 342 |
| | MHVDANTWATKFAVMVQQQDFENATTPGEVLTTHFTRTKQFVKDYYD |
| | RVNEVLYMDTSIMPFCNDTMLNAVMHRLVSVVREVLSFPLIYANPPE |
| | VRKEFDFENITTMKLLMPASFNNDTTREALMDADMDAALLQILRELY |
| | GPEHVVTLVFPMPQYDFSKLTDEQLVEVRWFILDLVRARLEECAVLS |
| | VDGVGASVSSHSSVCEAVITNRTETVVSHPPFNIQSEYEVFVPSRYK |
| | FNASLCLDGIDWAVLEELIKNYTEENKPRHKSACDRSCIIGLAVLAA |
| | LVLTALIAVMVVLTSKRRRLAAVVAPVHPKIKSTLDEDEEEMETTNP |
| | LEVKDEQRARDMY (SEQ ID NO: 38) |
| TcYC6_0064040 | >TcYC6_0064040\|Trypanosoma cruzi Y C6\|FLA1- |
| | binding protein\|protein\|length = 712 |
| | MSRFQSLLFALFAGFLFSFTASVVVAMPLRYMVETVSGITGSIGHVN |
| | GGPGTSLLTRPSAICQGRNEDELLFGTQGYFRNFSRSTKMTGILLGD |
| | GTAQILDGTWSQARIDGPRGCVRGIFNQKMIVYFVEGQSSLRYFTSN |
| | YVHTVTISINLSFTDVKLYEGKLYITEQTKDEVWGCDIDADGAPVSC |
| | ALKTGFKCDYGKYHGITVTKLGVFVVGESAAGICHFDMHGNKISVLG |
| | GNYIDVFSLPSDELYIMSYTELLHLRVIGSAMVVEKFAGRSDATCPP |
| | LTDGYDFTLCKNLRLFVIEQSEMYLATTLNTVRSVTLPPAIVWIELP |
| | PPPLPIGYPNDNEVMKKIIQLMNEELNKHLGTNGTYVSQETMHVDAN |
| | TWATKFAVMVQQQDFENATTPGEVLTTHFTRTKQFVKDYYDRVNEVL |
| | YMDTSIMPFCNDTMLNAVMHRLVSVVREVLSFPLIYANPPEVRKEFD |
| | FENITTMKLLMPASFNNDTTREALMDADMDAALLQILRELYGPEHVV |
| | TLVFPMPQYDFSKLTDEQLVEVRWFILDLVRARLEECAVLSVDGVGA |
| | SVSSHSSVCEAVITNRTETVVSHPPFNIQSEYEVFVPSRYKFNASLC |
| | LDGIDWAVLEELIKNYTEENKPRHKSACDRSCIIGLAVLAALVLTAL |
| | IAVMVVLTSKRRRLAAVVAPVHPKIKSTLDEDEEEMETTNPLEVKDE |
| | QRARDMY (SEQ ID NO: 39) |
| Tc_MARK_6973 | >Tc_MARK_6973\|Trypanosoma cruzi marinkellei |
| | strain B7\|hypothetical protein, conserved\| |
| | protein\|length = 599 |
| | QGIINDKTIIYFVEGQSSLRYITADYVHTVTISTKLSFTDVKLYGGK |
| | LYMTEQTKDEIWVCDIDMNGVPVTCVLQNGFKCDYGKYHGITVTKLG |
| | VFVVGESASGICHFDMHGNKISVLGGNYVDVYSLPTDTLFVMSFTEL |
| | LHLRVVGSMMVVEKFAGRVDATCPPLLDGYDFTLCMNLRLFVIDQNE |
| | MYLATKLNTVRSITLPPVVVWMELPPPPFPLGFPDDDEKKENVMHKI |
| | IQLMNEELNAHLRTQGTYVSLETMQVNDDTWNTKFAVMVQQQDFESA |
| | TTPAEVLSTDFVQTKKFIMDYYNRVNEVLYMDTSIIPFCDQTMLLQM |
| | MHKLVAIVRNVLGFPLIYANPPEALKDFHIENITIMKLLMPASFNND |
| | TTRDALMDTDMDAALLQVLRELYGPDHVVTLIFPMPKYEFSKLTDEQ |
| | LIQVRWFILDLVRARLAECEILSVDSMDTSSQGTMCEATITNRTETV |
| | VPTPPFNLQSEYEVFVPSRYKFNVSVCLDGIDWVALEELIANYTEEN |
| | KPRHKSACDRSCIIGLAVLTALVLTALIAVLVVLTSKRRRLAAVVAP |
| | VHPKFKSTLDEEEEEEMETSNPLEVKEEERTRDTY (SEQ ID NO: |
| | 40) |

Trypanosoma evansi

| | |
|---|---|
| TevSTIB805.5.5150 | >TevSTIB805.5.5150\|Trypanosoma evansi strain |
| | STIB 805\|hypothetical protein, conserved\| |
| | protein\|length = 807 |
| | MCFIFGVEMSNLAKRPMSLRKLPQLFLLIMIGIAFVAVECIGTPVKLP |
| | RRVDTVAGQFGVEGETNGYPNTTRLTEPYALCRGRTNDEILVGSSNSF |
| | RNYSRKTKETGTYLRYNVGDSVISGSSTINKPRSCVRRGSGNHTIIYF |
| | VDDQKDIKYIVGDDVSSFSVPTRGSLNAVAVHEGSLYVTDQNNKSVWK |
| | CGLGGAGKPQSCEEKKFTSVTLDAKPEGIAVTSKGIFVTARDSSNKGA |
| | LLWLDMSGSNRKGNVSGGFVDVESTESGMLYAATEKELYTVTATDTSL |
| | SVTLFAGKNTSQCYFPTNGEDIVLCDNSRLLVIEEYEMYVTSRAKHTM |
| | RALTLPPVNLTAIFRGRPAPVGYPNTTIMEQFVASLTEDVNKALGTND |
| | SYVDPDSVRVDPDTWETNYTVFVQQTRFDNTTEEKLRSLTYTQTDKTV |
| | DEYYGLTDEYVYIDTVLVPFCDDASLVTVDGRRAGYADKPITFGSDVA |
| | ENVTAVKLLMPHSFKNATTPKQLSAANLTDFAHNLVKDLRASDTRVDI |
| | TFPDPPFNFSAVVPEREQEVRWFVHGKVMKQLEICERLGSQGDAAVIA |
| | AAADATARGKANVTLNTSGVKANDTGVGPNTTNTAGGANTTANVATNG |
| | TANVIVNPSTNATPTGTTNASVTNTTERAVPVVAPTQPSNGYAECRSA |
| | ITNRTETQNMEPPYDRKHRYEVFLPKKYDFNVSWCVDIIDWRDLDEML |
| | NNRTDEVVEKSLSWCGHGCIIAFAVVGSLIAACLVVLAVVLTSKRRRL |
| | AAVVAPPRPKFVSTVEDDEEDRVSNIGMPLTDGKGTTAP (SEQ ID |
| | NO: 41) |
| TevSTIB805.5.5170 | >TevSTIB805.5.5170\|Trypanosoma evansi strain |
| | STIB 805\|hypothetical protein, conserved\| |
| | protein\|length = 818 |
| | MCFIFGVEMSNLAKRPMSLRKLPQLLLLIMIGIAFVAVECIGAPVKLP |
| | RRVDTVAGQFGFDGTTDGSSNVSMLSSPYALCRGRTNDEILVGSSNSF |
| | RNYSRKTKETGTFLRGGPTGGLVSADAKISKPRSCVRRGSGNHTIIYF |
| | VDDQNGLKYINDNEIQHVTVGNGLSLTSVAIYEKDLYVTDQNNKSVWR |
| | CNVGGAGKPQNCEEKKFTGLTFTAKPEGIAVTSKGIFVAARDSSNKGA |
| | LLWLDMNGGGSKGNVSGGFVDVFSTESGMLYAATEKELYTVTATDTSF |
| | SVTSFAGKNTSSCYSHANGEDIVLCDNSRLLVIEEYEMYVTSKEKHTM |
| | RALTLPPVNLTAIFRGRPAPVGYPNTTIMEQFVASLTEDVNKALGTND |
| | SYVDPDSVRVDPDTWETNYTVFVQQTRFDNTTEEKLRSLTYTQTDKTV |
| | DEYYGLTDEYVYIDTVLVPFCDDASLVTIQRALAREAGRALNFSLIYA |
| | DKPITFGSDVAENVTAVKLLMPHSFKNATTPKQLSAANLTDFAHNLVK |
| | DLRASDTRVDITFPDPPFNFSAVVPEREQEVRWFVHGKVMKQLEICER |
| | LGSQGDAAVIAAAADATARGKANVTLNTSGVKANDTGVGPNTTNTAGG |
| | ANTTANVVANGTANVIVNPSTNATPTGTSNASVTNTTERAVPVVAPTQ |
| | PSNGYAECRSAITNRTETQNMEPPYDRKHRYEVFLPKKYDFNVSWCVD |
| | IIDWRDLDEMLNNRTDEVVEKSLSWCGHGCIIAFAVVGSLIAACLVVL |
| | AVVLTSKRRRLAAVVAPPRPKFVSTVEDDEEDRVSNIGVPLTDGKGTT |
| | AP (SEQ ID NO: 42) |
| TevSTIB805.8.4170 | >TevSTIB805.8.4170\|Trypanosoma evansi strain |
| | STIB 805\|hypothetical protein, conserved\| |
| | protein\|length = 750 |
| | MPLWKQTNCEVETMNVREVVGTVHLGYVSQMLLLVATVETIVVRSGAA |
| | PIELKRHVTTVAGKYGHIGDKDGFPGMSELSSPHAMCRGRNSDEILLG |
| | TVDRFRAFSRKNRETTTITAWETDEDQNSGSRSVKVDKPRACVQWTVG |
| | DSTFVYFVESMGEVKYFKDSGVFSHDVVRNGSLTGVALYGNHLYLTEQ |
| | NTNTVWTCEVGSDGDPIACHSHVALSAKCSIYGPIGIAATQQGIFVVA |
| | RGPAKQGTICWFDLQGHKIAEVDGEYVDITSTRSGDLLAATQNELHRV |
| | STDGNKLTTKRFAGGSTNSCLPNTEGDDTLLCEITRLLVVTEYEMYVT |
| | SEKKSVLRSVTLPPVYVQGVFPGRPLPVGYPDKDIMEWIVGNLTEDIN |
| | TALGTTESIVASSSVHVDSTTWLTNFTAGVQQPDFDDEKTEQALHKSN |
| | YEHTKEAADEYYNLTDEQVYMDSTMVPYCNRLSLDALRRKLAKEAGEV |
| | LNFTLIYADMPLKAESSDAGNITTVKLLMPASFNNTVTHDLLSDANLT |
| | ETAHSFIKYLRSSDTHVDVTFSNPPFNESSLTPDEEQEVRWYIHDEVM |
| | NQIKKCEERSTGRSMARREEVGDYSRTTIATALDSNVTGVCQSTITNR |
| | TVSLFYQPPYVEMSLYEVFIPGNYTFDVSECVGEIDWQDLNDHLNNDT |
| | VRPTTEKAPKCGRVCLIIIAVVCALIVAVLIVLAVVFTSKRRRLAAVV |
| | APARPKFVSTLDEDEQDYASAYGNKERVEQ (SEQ ID NO: 43) |
| TevSTIB805.8.4220 | >TevSTIB805.8.4220\|Trypanosoma evansi strain |
| | STIB 805\|hypothetical protein, conserved\| |
| | protein\|length = 750 |
| | MPLWKQTNCEVETMNVREVVGTVHLGYVSQMLLLVATVETIVVRSGAA |
| | PIELKRHVTTVAGKYGHIGDKDGFPGMSELSSPHAMCRGRNSDEILLG |
| | TVDRFRAFSRKNRETTTITAWETDEDQNSGSRSVKVDKPRACVQWTVG |
| | DSTFVYFVESMGEVKYFKDSGVFSHDVVRNGSLTGVALYGNHLYLTEQ |
| | NTNTVWTCEVGSDGDPIACHSHVALSAKCSIYGPIGIAATQQGIFVVA |
| | RGPAKQGTICWFDLQGHKIAEVDGEYVDITSTRSGDLLAATQNELHRV |
| | STDGNKLTTKRFAGGSTNSCLPNTEGDDTLLCEITRLLVVTEYEMYVT |
| | SEKKSVLRSVTLPPVYVQGVFPGRPLPVGYPDKDIMEWIVGNLTEDIN |
| | TALGTTESIVASSSVHVDSTTWLTNFTAGVQQPDFDDEKTEQALHKSN |
| | YEHTKEAADEYYNLTDEQVYMDSTMVPYCNRLSLDALRRKLAKEAGEV |
| | LNFTLIYADMPLKAESSDAGNITTVKLLMPASFNNTVTHDLLSDANLT |
| | ETAHSFIKYLRSSDTHVDVTFSNPPFNFSSLTPDEEQEVRWYIHDEVM |
| | NQIKKCEERSTGRSMARREEVGDYSRTTIATALDSNVTGVCQSTITNR |
| | TVSLFYQPPYVEMSLYEVFIPGNYTFDVSECVGEIDWQDLNDHLNNDT |
| | VRPTTEKAPKCGRVCLIIIAVVCALIVAVLIVLAVVFTSKRRRLAAVV |
| | APARPKFVSTLDEDEQDYASAYGNKERVEQ (SEQ ID NO: 44) |

Examples of Leishmania species include: Leishmania aethiopica, Leishmania amazonensis, Leishmania arabica, Leishmania aristidesi, Leishmania donovani, Leishmania forattinii, Leishmania gerbilli, Leishmania infantum, Leishmania killicki, Leishmania major, Leishmania mexicana, Leishmania pifanoi, Leishmania tropica, Leishmania turanica, Leishmania venezeulensis, Leishmania waltoni, Leishmania enriettii, Leishmania macropodum, Leishmania martiniquensis, Leishmania orientalis, Leishmania adleri, Leishmania agamae, Leishmnania ceramodactyli, Leishmania gulikae, Leishmania gymnodactyli, Leishmania helioscopi, Leishmania hemidactyli, Leishmania hoogstraali, Leishmania nicollei, Leishunania platycephala, Leishmania phrynocephali, Leishmania senegalensis, Leishmania sofieFi, Leishmania tarentolae, Leishmania zmeevi, Leishmania zuckermani, Leishmania braziliensis, Leishmania guyanensis, Leishmania lainsoni, Leishmania lindenbergi, Leishmania naiffi, Leishmania panamensis, Leishmania peruviana. Leishmania shawi and Leishmania utingensis.

In one embodiment, the trypanosomal vaccine is a Leishmania aethiopica, Leishmania amazonensis, Leishmania braziliensis, Leishmania donovani, Leishmania infantum, Leishmania major, Leishmania mexicana, Leishmania panamensis or Leishmania tropica vaccine.

Examples of FLA1 binding proteins from Leishmania aethiopica, Leishmania amazonensis, Leishmania braziliensis, Leishmania donovani, Leishmania infantum, Leishmania major, Leishmania mexicana, Leishmania panamensis or Leishmania tropica include the following:

Leishmania aethiopica

| | |
|---|---|
| LAEL147_000137700 | >LAEL147_000137700\|Leishmania aethiopica |
| | L147\|hypothetical protein, conserved\| |
| | protein\|length = 756 |
| | MGRCIRRVPAAAAAALLLALVAAAAVSTTTARAYDHAGITVAGAIM |
| | VGQNLQGKAGASRILNPFAICANFDTADVEDTTLLIGGASYFFTLN |
| | RYSTYLGFWYGQGSVNLNSGPIDKVRLTGVFGCVTLRPNSSNSLVT |
| | STVYYVQNDGMLYWVSNSVVYLTPVKHGISFVDVTVHDNNVYLLST |
| | QNHIYRCGIGAGGAVVGSACTQITLTGSTKFDQLITTPSDFRGFVV |
| | SSCGIFIAPNSDLYWFNLSGVFIAKSAGVTFVDIKLTSNRDTANRG |
| | TPVLMAASTSAVYAVTASSATISYTLVSGKETKSCNPALNNVDSDT |
| | SPTFCGIARIYPLSTDMVYMTTGGASVVRAIIVGNTTISDTITRTP |
| | FPVYFLDNPSIIPLILDGMNYELVGNSNIPFPYVAINHSTPEVDDG |
| | TWDTTFSVDVSNRFFSTVSSAAVVSTPFMGSLHGLQAYYNRTNQIL |
| | FGDPNVLPMCNLTKMQMIERAVAADARAALQYPYIYTSKAQNFTVN |
| | AHPNLTLLKLLMPYPFGEILNESGFFENTTTPAALANVHFNTTMLA |
| | AVRNAYTPDFVYDCIFAGNAFPFHILTAAQQQLVRWIIYTAIQEQL |
| | AKCAENSPSYTGSDSSSSDSHDDMVPGCVPRVGIGNLTELVMPGMP |
| | YSNYNITVFIPEGLHYNFSISRCLDGTDWTNVTDYLQHATTPRTRQ |
| | CGTGCIVSIAVVSAVVAAILVVAIVIATSKRRRLATVVAPAFTVEP |
| | KFASTLDMSSEEGSRNPLNG (SEQ ID NO: 45) |

Leishmania amazonensis

| | |
|---|---|
| LAMA_000162600 | >LAMA_000162600\|Leishmania amazonensis |
| | MHOM/BR/71973/M2269 hypothetical protein, |
| | conserved\|protein\|length = 755 |
| | MGRSIRRVSAAAAALLMALVAAAAVAPTTARAYDHAGITVAGALM |
| | VGQNLQGTAATSRILNPFAICANFDTADVEDTTLLIGGASYFFTL |
| | NRYSTYLGFWYGQGSMNLNSGPIDKVRLTGVFGCVTLRPNPSNGL |
| | PTSIVYYVQNDGFLYWVSNSIVYLTQVESGISLFDVTVYNNSVYL |
| | LSAQNVIYRCGIGAGGAVVGSACTQILLTGSPAFHQLIAVSSDFR |
| | GFAVSASGIVVAPTADLFWFNLSGAFISKSAGVTFVDAKFTTNRD |
| | TANRGAPVLMAASTSAVYTVATSGPSITYTLVSGEETGRCNPALN |
| | NVDSDTSPTFCGIARIYPLSTDMVYMTTGGASVVRAILVGNTTVH |
| | DTITRTPFPVYFLDNSSIMPLMLDGMNYELVANSDIPFPYVAINE |
| | FTPEVGDSTWDTSFSVDVSNRFFSTASSAAVISTPFMGSLHGLQA |
| | YYNRTNQILFGDPNVLPMCNLTKMHMIERAVAADARAALQYPYIY |
| | TSRAQNFTVNAQPNLTLLKLLMPYPFGEILNESGFFENTTTPAAL |
| | ANVHFNKTMLAAVRNAYAPDFVYDSIFAGNAFPFHILTAAQQQVV |
| | RWVIYMAIQEQLAKCAENTPSYPDSDSSSSDSHDDMVPGCVPRVG |
| | ISNLTEQMIPGLPYSNFNITFFIPESLHYTFSISRCLDGTDWTNV |
| | TDYLQNATITSTRECGTGCIVSIAVASAVVAAILVVVIVIVTSKR |
| | RRLATVVAPALTVEPKFASTLDVTSEEGSRNPLNG (SEQ ID |
| | NO: 46) |

Leishmania braziliensis

| | |
|---|---|
| LBRM2903_100015700 | >LBRM2903_100015700\|Leishmania braziliensis |
| | MHOM/BR/75/M2903\|hypothetical protein, |
| | conserved\|protein\|length = 756 |
| | MGRCVYRVSSAAATLLTVLIAAVTVAATTARAYDHAGVTVAGALL |
| | VGQNEEGKLGTNRILNPFALCANFDTTDVTDTTLLIGGASYFFTF |
| | DRHSTYLSFWYGQGSMNLNSGPIDQVRLTGVFGCTTVRTTSSSGS |
| | PISAVYYVQNDGFLYWVMNSVVYVTLVKNGISLFDVTVYKGNLYL |
| | LSAQNRIYKCLIGPGGAVTGSACTQVMLTGSTAYANLSETSTSEF |
| | KGFAVSSAGIFIAPSSSLYWFNLAGGYIASTTTAVVFVDVKFTSN |
| | RDTANPGIPTLMAASTSAVYRVSTAGTSITYTLIAGKETATCNLA |
| | LDNVDSLTDPSFCGIARIYPLSLDVVYMTTAGASVVRAIVVSNTT |
| | IRDTITRTPFPLYFLDRASTIPVLLDGMNYEIVGHSNVPFPYVAI |
| | DQSTPEVNDTTWDTSFGVDISNRFFSMASSAAVTSTPFMGSLHGL |
| | ETYYNRTNQIIFGDPNVLPMCNLTKMLMIERAVATAARAALKYPY |
| | IYTSNAQNFTVNAQPNLTLVKLLMPYAFGEILNELGFFENTTTAA |
| | ALAAVQFNTTMLAAVRSAYMMDRVYDCIFSGNAYPFHVLTAAQQQ |
| | EVRWIIYSAIQNQLARCNQSIPYLVPDSNSSNSYNNMAPGCVPRI |
| | GINNLTEMLLPGLPYSNFNITVFIPESLYYNFGISRCLDGTDWTD |
| | VMGYLINATTRNNRKCNTGCIVGIAVASALVASFLVVAIVIMTSK |
| | RRRLATVVAPAATSEPKFISTLDMTSEEGSRNPLTR (SEQ ID |
| | NO: 47) |
| LbrM.10.0760 | >LbrM.10.0760\|Leishmania braziliensis MHOM/ |
| | BR/75/M2904\|FLA1-binding protein\|protein\| |
| | length = 756 |
| | MGRCVYRVSSAAATLLTVLIAAVTVAATTARAYDHAGVTVAGALL |
| | VGQNEEGKLGTNRILNPFALCANFDTTDVTDTTLLIGGASYFFTF |
| | DRHSTYLSFWYGQGSMNLNSGPIDQVRLTGVFGCTTVRTTSSSGS |
| | PISTVYYVQNDGFLYWVMNSVVYVTLVKNGISLFDVTVYKGNLYL |
| | LSAQNRIYKCLIGPGGAVTGSACTQVMLAGSTAYANLSETSTSEF |
| | KGFAVSSAGIFIAPSSSLYWFNLAGGYIASTTTAVVFVDVKFTSN |
| | RDTANPGIPTLMAASTSAVYRVSTAGTSITYTLIAGKETATCNLA |
| | LDNVDSLTDPSFCGIARIYPLSLDVVYMTTAGASVVRAIVVSNTT |
| | IRDTITRTPFPLYFLDRASTIPVLLDGMNYEIVGHSNVPFPYVAI |
| | DQSTPEVNDTTWDTSFGVDISNRFFSMASSAAVTSTPFMGSLHGL |
| | ETYYNRTNQIIFGDPNVLPMCNLTKMLMIERAVATAARAALKYPY |
| | IYTSNAQNFTVNAQPNLTLVKLLMPYAFGEILNELGFFENTTTAA |
| | ALAAVQFNTTMLAAVRSAYMMDRVYDCIFSGNAYPFHVLTAAQQQ |
| | EVRWIIYSAIQNQLARCNQSIPYLGPDSNSSNSYNNMAPGCVPRI |
| | GINNLTEMLLPGLPYSNFNITVFIPESLYYNFGISRCLDGTDWTD |
| | VMGYLINATTRNNRKCNTGCIVGIAVASALVASFLVVAIVIMTSK |
| | RRRLATVVAPAATSEPKFISTLDMTSEEGSRNPLTR (SEQ ID |
| | NO: 48) |
| LbrM.10.2.000760 | >LbrM.10.2.000760\|Leishmania braziliensis |
| | MHOM/BR/75/M29042019\|hypothetical protein, |
| | conserved\|protein\|length = 756 |
| | MGRCVYRVSSAAATLLTVLIAAVTVAATTARAYDHAGVTVAGALL |
| | VGQNEEGKLGTNRILNPFALCANFDTTDVTDTTLLIGGASYFFTF |
| | DRHSTYLSFWYGQGSMNLNSGPIDQVRLTGVFGCTTVRTTSSSGS |
| | PISTVYYVQNDGFLYWVMNSVVYVTLVKNGISLFDVTVYKGNLYL |
| | LSAQNRIYKCLIGPGGAVTGSACTQVMLAGSTAYANLSETSTSEF |
| | KGFAVSSAGIFIAPSSSLYWFNLAGGYIASTTTAVVFVDVKFTSN |
| | RDTANPGIPTLMAASTSAVYRVSTAGTSITYTLIAGKETATCNLA |
| | LDNVDSLTDPSFCGIARIYPLSLDVVYMTTAGASVVRAIVVSNTT |
| | IRDTITRTPFPLYFLDRASTIPVLLDGMNYEIVGHSNVPFPYVAI |
| | DQSTPEVNDTTWDTSFGVDISNRFFSMASSAAVTSTPFMGSLHGL |
| | ETYYNRTNQIIFGDPNVLPMCNLTKMLMIERAVATAARAALKYPY |
| | IYTSNAQNFTVNAQPNLTLVKLLMPYAFGEILNELGFFENTTTAA |
| | ALAAVQFNTTMLAAVRSAYMMDRVYDCIFSGNAYPFHVLTAAQQQ |
| | EVRWIIYSAIQNQLARCNQSIPYLGPDSNSSNSYNNMAPGCVPRI |
| | GINNLTEMLLPGLPYSNFNITVFIPESLYYNFGISRCLDGTDWTD |
| | VMGYLINATTRNNRKCNTGCIVGIAVASALVASFLVVAIVIMTSK |
| | RRRLATVVAPAATSEPKFISTLDMTSEEGSRNPLTR (SEQ ID |
| | NO: 49) |

Leishmania donovani

| | |
|---|---|
| LdBPK_100670.1 | >LdBPK_100670.1\|Leishmania donovani |
| | BPK282A1\|FLA1-binding protein\|protein\| |
| | length = 756 |
| | MGRCIRRVSSAAAAALLIALAAAAAVATTTARAYDHAGITVAGALL |
| | VGQNLQGKAGASRILNPFAICADFDTADVEDTTLLIGGASYFFSFN |
| | RYSTYLGFWYGQGSMNLNSGPIDKVRLTGVFGCVTLRPNSSNGLVT |
| | STVYYVQNDGMLYWVSNSVVYLTQVKHGISFVDVTVHDNNVYLLST |
| | QNGIYQCGIGAGGAVVGSACTQITLTGSTEFHQLIPISSDFRGFAV |
| | SSSGIFITPTSDLYWFNLSGAFIAKSVGVTFVDTKFTSSRDTANRG |
| | TSVLMAASTSAVYTVTTSDATISYALVSGKEVKSCNPALNNVDSDT |
| | SPTFCGIARIYPLSTDMVYMTTGGASVVRAIIVSNTTISDTITRTP |
| | FPVYFLDNSSIMPLILDGMNYELVSNSNIPFPYVAINHSTPKVDDS |
| | TWDTIFSVDVSNRFFSTVSRAAVIGTPFMSSLHGLQAYYNRTNHIL |
| | FGDPNVLPMCNLTKMQMIERAVAADARAALQYPYIYTSKAQNFTVN |
| | AHPNLTLLKLLMPYPFGEILNESGFFENTTTPASLANVHFNTTMLA |
| | AVRNAYTPDFVYDCIFAGNAFPFHILTAAQQQLVRWIIYTAIQEQL |
| | AKCAENSPSYTGSGSSSSDSHDDMVPGCVPRVGIDNLTELVMPGMP |
| | YSNYNITVFIPESLHYNFSISRCLDGTDWTNVTDYLQNATTTRTRK |
| | CGTGCIVSIAVVSAVVAAILVVAIVIATSKRRRLATVVAPALTVEP |
| | KFASTLDMGSEEGSRNPLNG (SEQ ID NO: 50) |
| LdCL_100013500 | >LdCL_100013500\|Leishmania donovani CL-SL\| |
| | hypothetical protein\|protein\|length = 756 |
| | MGRCIRRVSSAAAAALLIALAAAAAVATTTARAYDHAGITVAGALL |
| | VGQNLQGKAGASRILNPFAICADFDTADVEDTTLLIGGASYFFSFN |
| | RYSTYLGFWYGQGSMNLNSGPIDKVRLTGVFGCVTLRPNSSNGLVT |
| | STVYYVQNDGMLYWVSNSVVYLTQVKHGISFVDVTVHDNNVYLLST |
| | QNGIYQCGIGAGGAVVGSACTQITLTGSTEFHQLIPISSDFRGFAV |
| | SSSGIFITPTSDLYWFNLSGAFIAKSVGVTFVDTKFTSSRDTANRG |
| | TSVLMAASTSAVYTVTTSDATISYALVSGKEVKSCNPALNNVDSDT |
| | SPTFCGITRIYPLSTDMVYMTTGGASVVRAIIVSNTTISDTITRTP |
| | FPVYFLDNSSIMPLILDGMNYELVSNSNIPFPYVAINHSTPKVDDS |
| | TWDTIFSVDVSNRFFSTVSRAAVIGTPFMSSLHGLQAYYNRTNHIL |
| | FGDPNVLPMCNLTKMQMIERAVAADARAALQYPYIYTSKAQNFTVN |
| | AHPNLTLLKLLMPYPFGEILNESGFFENTTTPASLANVHFNTTMLA |
| | AVRNAYTPDFVYDCIFAGNAFPFHILTAAQQQLVRWIIYTAIQEQL |
| | AKCAENSPSYTGSGSSSSDSHDDMVPGCVPRVGIDNLTELVMPGMP |
| | YSNYNITVFIPESVHYNFSISRCLDGTDWTNVTDYLQNATTTRTRK |
| | CGTGCIVSIAVVSAVVAAILVVAIVIATSKRRRLATVVAPALTVEP |
| | KFASTLDMGSEEGSRNPLNG (SEQ ID NO: 51) |
| LdBPK.10.2.000670 | >LdBPK.10.2.000670\|Leishmania donovani strain |
| | LV9\|hypothetical protein, conserved\|protein\| |
| | length = 756 |
| | MGRCIRRVSSAAAAALLIALAAAAAVATTTARAYDHAGITVAGALL |
| | VGQNLQGKAGASRILNPFAICADFDTADVEDTTLLIGGASYFFSFN |
| | RYSTYLGFWYGQGSMNLNSGPIDKVRLTGVFGCVTLRPNSSNGLVT |
| | STVYYVQNDGMLYWVSNSVVYLTQVKHGISFVDVTVHDNNVYLLST |
| | QNGIYQCGIGAGGAVVGSACTQITLTGSTEFHQLIPISSDFRGFAV |
| | SSSGIFITPTSDLYWFNLSGAFIAKSVGVTFVDTKFTSSRDTANRG |
| | TSVLMAASTSAVYTVTTSDATISYALVSGKEVKSCNPALNNVDSDT |
| | SPTFCGIARIYPLSTDMVYMTTGGASVVRAIIVSNTTVSDTITRTP |
| | FPVYFLDNSSIMPLILDGMDYELVSNSNIPFPYVAINHSTPKVDDS |
| | TWDTIFSVDVSNRFFSTVSRAAVIGTPFMSSLHGLQAYYNRTNHIL |
| | FGDPNVLPMCNLTKMQMIERAVAADARAALQYPYIYTSKAQNFTVN |
| | AHPNLTLLKLLMPYPFGEILNESGFFENTTTPASLANVHFNTTMLA |
| | AVRNAYTPDFVYDCIFAGNAFPFHILTAAQQQLVRWIIYTAIQEQL |
| | AKCAENSPSYTGSGSSSSDSHDDMVPGCVPRVGIDNLTELVMPGMP |
| | YSNYNITVFIPESLHYNFSISRCLDGTDWTNVTDYLQNATTTRTRK |
| | CGTGCIVSIAVVSAVVAAILVVAIVIATSKRRRLATVVAPALTVEP |
| | KFASTLDMGSEEGSRNPLNG (SEQ ID NO: 52) |

Leishmania infantum

| | |
|---|---|
| LINF_100013000 | >LINF_100013000\|Leishmania infantum JPCM5\| |
| | hypothetical protein - conserved\|protein\| |
| | length = 756 |
| | MGRCIRRVSSAAAAALLIALAAAAAVATTTARAYDHAGITVAGALL |
| | VGQNLQGKAGASRILNPFAICADFDTADVEDTTLLIGGASYFFSFN |
| | RYSTYLGFWYGQGSMNLNSGPIDKVRLTGVFGCVTLRPNSSNGLVT |
| | STVYYVQNDGMLYWVSNSVVYLTQVKHGISFVDVTVHDNNVYLLST |
| | QNGIYQCGIGAGGAVVGSACTQITLTGSTEFHQLIPISSDFRGFAV |
| | SSSGIFITPTSDLYWFNLSGAFIAKSVGVTFVDTKFTSSRDTANRG |
| | TSVLMAASTSAVYTVTTSDATISYALVSGKEVKSCNPALNNVDSDT |
| | SPTFCGIARIYPLSTDMVYMTTGGASVVRAIIVSNTTVSDTITRTP |
| | FPVYFLDNSSIMPLILDGMNYELVSNSNIPFPYVAINHSTPKVDDS |
| | TWDTIFSVDVSNRFFSTVSRAAVIGTPFMSSLHGLQAYYNRTNHIL |
| | FGDPNVLPMCNLTKMQMIERAVAADARAALQYPYIYTSKAQNFTVN |
| | AHPNLTLLKLLMPYPFGEILNESGFFENTTTPASLANVHFNTTMLA |
| | AVRNAYTPDFVYDCIFAGNAFPFHILTAAQQQLVRWIIYTAIQEQL |
| | AKCAENSPSYTGSGSSSSDSHDDMVPGCVPRVGIDNLTELVMPGMP |
| | YSNYNITVFIPESLHYNFSISRCLDGTDWTNVTDYLQNATTTRTRK |
| | CGTGCIVSIAVVSAVVAAILVVAIVIATSKRRRLATVVAPALTVEP |
| | KFASTLDMGSEEGSRNPLNG (SEQ ID NO: 53) |

Leishmania major

| | |
|---|---|
| LmjF.10.0620 | >LmjF.10.0620\|Leishmania major strain |
| | Friedlin\|FLA1-binding protein\|protein\| |
| | length = 757 |
| | MGRCIRRVSALKAAAALLLALVAAAAVATTTARAYDHAGITVAGAI |
| | LVGQNLQGKAGASRILNPFAICANFDTTDAEDTTLLIGGASYFFTL |
| | NRYSTYLGFWYGQGSMNLNNGPIDKVRLTGVFGCVTLRPNSSNGLV |
| | TSTVYYVQNDGMLYWVSNSVVYLTQVKYGISFVDVTVHDNNVYLLS |
| | NQNEIYWCGIGAGGTVVGSACTHITLTGSTEFHQLITVSSDFRGFA |
| | VSSSGIFIAPTSDLYWFNLSGVFIAKSAGVTFVDTKFTSSRDTVNR |
| | ETPVLMAASTSAVYSVTTSGATISYTLVSGKETKSCNPALSNVDSD |
| | TSPTFCGIARIYPLNTDTVYMTTGGASVVRAIIVSNTTISDTITRT |
| | PFPVYFLDNSSIIPLILDGMNYELVGNSNIPFPYVAINHSTPEVDD |
| | STWDTTFSVDVSNRFFSSVSSAAVVSTPFMGSLHGLQAYYNRTNQI |
| | LFGDPNVLPMCNLTKMLMIERAVAADARAALQYPYIYTSKAQNFTV |
| | NAHPNLTLLKLLMPYPFGEILNESGFFENTTTPAALANVHFNTTML |
| | AAVRNAYTPDFVYDCIFAGNAFPFYILTAAQQQLVRWIIYTAIQEQ |
| | LAKCAENNPSYTGSDSSSSDSHDDMVPGCVPRVGISNLTELVMPGM |
| | PYSNYNIAVFIPESLHYNFSISRCLDGTDWTNVTEYLQQATVARTR |
| | KCGTGCIVSIAVVSAVVAAILVVAIVIATSKRRRLATVVAPALTVE |
| | PKFASTLDVSSEEGSRNPLNG (SEQ ID NO: 54) |
| LMJLV39_100012300 | >LMJLV39_100012300\|Leishmania major strain |
| | LV39c5\|hypothetical protein, conserved\| |
| | protein\|length = 757 |
| | MGRCIRRVSALKAAAALLLALVAAAAVATTTARAYDHAGITVAGAI |
| | LVGQNLQGKAGASRILNPFAICANFDTTDAEDTTLLIGGASYFFTL |
| | NRYSTYLGFWYGQGSMNLNNGPIDKVRLTGVFGCVTLRPNSSNGLV |
| | TSTVYYVQNDGMLYWVSNSVVYLTQVKYGISFVDVTVHDNNVYLLS |
| | NQNEIYWCGIGAGGTVVGSACTHITLTGSTEFHQLITVSSDFRGFA |
| | VSSSGIFIAPTSDLYWFNLSGVFIAKSAGVTFVDTKFTSSRDTVNR |
| | ETPVLMAASTSAVYSVTTSGATISYTLVSGKETKSCNPALSNVDSD |
| | TSPTFCGIARIYPLNTDTVYMTTGGASVVRAIIVSNTTISDTITRT |
| | PFPVYFLDNSSIIPLILDGMNYELVGNSNIPFPYVAINHSTPEVDD |
| | STWDTTFSVDVSNRFFSSVSSAAVVSTPFMGSLHGLQAYYNRTNQI |
| | LFGDPNVLPMCNLTKMLMIERAVAADARAALQYPYIYTSKAQNFTV |
| | NAHPNLTLLKLLMPYPFGEILNESGFFENTTTPAALANVHFNTTML |
| | AAVRNAYTPDFVYDCIFAGNAFPFYILTAAQQQLVRWIIYTAIQEQ |
| | LAKCAENNPSYTGSDSSSSDSHDDMVPGCVPRVGISNLTELVMPGM |
| | PYSNYNIAVFIPESLHYNFSISRCLDGTDWTNVTEYLQQATVARTR |
| | KCGTGCIVSIAVVSAVVAAILVVAIVIATSKRRRLATVVAPALTVE |
| | PKFASTLDVSSEEGSRNPLNG (SEQ ID NO: 55) |
| LMJSD75_100012400 | >LMJSD75_100012400\|Leishmania major strain |
| | SD 75.1\|hypothetical protein, conserved\| |
| | protein\|length = 757 |
| | MGRCIRRVSALKAAAALLLALVAAAAVATTTARAYDHAGITVAGAI |
| | LVGQNLQGKAGASRILNPFAICANFDTTDAEDTTLLIGGASYFFTL |
| | NRYSTYLGFWYGQGSMNLNNGPIDKVRLTGVFGCVTLRPNSSNGLV |
| | TSTVYYVQNDGMLYWVSNSVVYLTQVKYGISFVDVTVHDNNVYLLS |
| | NQNEIYWCGIGAGGTVVGSACTHITLTGSTEFHQLITVSSDFRGFA |
| | VSSSGIFIAPTSDLYWFNLSGVFIAKSAGVTFVDTKFTSSRDTVNR |
| | ETPVLMAASTSAVYSVTTSGATISYTLVSGKETKSCNPALSNVDSD |
| | TSPTFCGIARIYPLNTDTVYMTTGGASVVRAIIVSNTTISDTITRT |
| | PFPVYFLDNSSIIPLILDGMNYELVGNSNIPFPYVAINHSTPEVDD |
| | STWDTTFSVDVSNRFFSSVSSAAVVSTPFMGSLHGLQAYYNRTNQI |
| | LFGDPNVLPMCNLTKMLMIERAVAADARAALQYPYIYTSKAQNFTV |
| | NAHPNLTLLKLLMPYPFGEILNESGFFENTTTPAALANVHFNTTML |
| | AAVRNAYTPDFVYDCIFAGNAFPFYILTAAQQQLVRWIIYTAIQEQ |
| | LAKCAENNPSYTGSDSSSSDSHDDMVPGCVPRVGISNLTELVMPGM |
| | PYSNYNIAVFIPESLHYNFSISRCLDGTDWTNVTEYLQQATVARTR |
| | KCGTGCIVSIAVVSAVVAAILVVAIVIATSKRRRLATVVAPALTVE |
| | PKFASTLDVSSEEGSRNPLNG (SEQ ID NO: 56) |

Leishmania mexicana

| | |
|---|---|
| LmxM.10.0620 | >LmxM.10.0620\|Leishmania mexicana MHOM/GT/ |
| | 2001/U1103\|FLA1-binding protein\|protein\| |
| | length = 755 |
| | MGRCIRRVSAAAAALLMALVAAAAVAPTTARAYDHAGITVAGALM |
| | VGQNLQGTAGTSRILNPFAICANFDTADVEDTTLLIGGASYFFTL |
| | NRYSTYLGFWYGQGSMNLNSGPIDKVRLTGVFGCVTLRPNPSNAL |
| | PTSIVYYVQNDGFLYWVSNSIVYLTQLESGISLFDVTVYNNSVYL |
| | LSAQNVIYRCGIGAGGAVVGSACTQIPLTGSPAFHQLITVSSDFR |
| | GFAVSASGIVVAPTADLFWFDLSGAFISKSAGVTFVDAKFTTNRD |
| | TANRGAPVLMAASTSAVYTVATSGASITYTLVSGEETGRCNPALN |
| | NVDSDTSPTFCGIARIYPLSTDMVYMTTGGASVVRAILVGNTTVR |
| | DTITRTPFPVYFLDNSSIMPLMLDGMNYELVANSDIPFPYVAINE |
| | STPEVADSTWDTSFSVDVSNRFFSTASSAAVTSTPFMGSLHGLQA |
| | YYNRTNQILFGDPNVLPMCNLTKMHMIERAVAADARAALQYPYIY |
| | TSRAQNFTVNAQPNLTLLKLLMPYPFGEILNESGFFENTTTPAAL |
| | ANVHFNKTMLAAVRNAYAPDFVYDSIFAGNAFPFHILTAAQQQVV |
| | RWVIYMAIQEQLAKCAENTPSYPGSDSSSSDSQDDMVPGCVPRVG |
| | ISNLTEQMIPGLPYSNFNITVFIPESLHYTFSISRCLDGTDWTNV |
| | TDYLQNATITTTRKCGTGCIVSIAVASAVVAAILVVAIVIVTSKR |
| | RRLATVVAPALTVEPKFASTLDATSEEGSRNPLNG (SEQ ID |
| | NO: 57) |

Leishmania panamensis

| | |
|---|---|
| LPAL13_100012300 | >LPAL13_100012300\|Leishmania panamensis |
| | MHOM/COL/81/L13\|hypothetical protein, |
| | conserved\|protein\|length = 756 |
| | MGRCVYRVSSAAATLLTVLIAAVTVAATTARAYDHAGVTVAGALL |
| | VGQNEEGKLGTNRILNPFALCANFDTTDVTDTALLIGGASYFFTF |
| | DRHSTYLSFWYGQGSMNLNSGPIDQVRLTGVFGCTTVRTTSSSGS |
| | PISTVYYVQNDGFLYWVMNSVVYVTLVKNGISLFDVTVYKGNLYL |
| | LSAQNSIYKCLIGPGGAVTGSACTQVMLAGSTAYANLSETSTSEF |
| | KGFAVSSAGIFIAPSSSLYWFNLAGGYIASTTTAVVFVDVKLTSN |
| | RDTANPGIPTLMAASTSAVYRVSTAGTSITYTLIAGKETATCNLA |
| | LDNVDSLTDPSFCGIARIYPLSLDVVYMTTAGASVVRAIVVSNTT |
| | IRDTITRTPFPLYFLDRASTIPVLLDGMNYEIVGHSNVPFPYVAI |
| | DQSTPEVNDTTWDTSFGVDISNRFFSMASSAAVTSTPFMGSLHGL |
| | ETYYNRTNQIIFGDPNVLPMCNLTKMLMIERAVATAARAALKYPY |
| | IYTSNAQNFTVNAQPNLTLVKLLMPYAFGEILNELGFFENTTTAA |
| | ALAAVQFNTTMLAAVRSAYMMDRVYDCIFSGNAYPFHVLTAAQQQ |
| | EVRWIIYSAIQNQLARCNQSIPYLGPDSNSSNSYNNMAPGCVPRI |
| | GINNLTEMLLPGLPYSNFNITVFIPESLYYNFGISRCLDGTDWTD |
| | VMGYLINATTRNNRKCNTGCIVGIAVASALVASFLVVAIVIMTSK |
| | RRRLATVVAPAATSEPKFISTLDMTSEEGSRNPLTR (SEQ ID |
| | NO: 58) |
| LPMP_100580 | >LPMP_100580\|Leishmania panamensis strain |
| | MHOM/PA/94/PSC-1\|hypothetical protein\| |
| | protein\|length = 756 |
| | MGRCVYRVSSAAATLLTVLIAAVTVAATTARAYDHAGVTVAGALL |
| | VGQNEEGKLGTNRILNPFALCANFDTTDVTDTALLIGGASYFFTF |
| | DRHSTYLSFWYGQGSMNLNSGPIDQVRLTGVFGCTTVRTTSSSGS |
| | PISTVYYVQNDGFLYWVMNSVVYVTLVKNGISLFDVTVYKGNLYL |
| | LSAQNSIYKCLIGPGGAVTGSACTQVMLAGSTAYANLSETSTSEF |
| | KGFAVSSAGIFIAPSSSLYWFNLAGGYIASTTTAVVFVDVKLTSN |
| | RDTANPGIPTLMAASTSAVYRVSTAGTSITYTLIAGKETATCNLA |
| | LDNVDSLTDPSFCGIARIYPLSLDVVYMTTAGASVVRAIVVSNTT |
| | IRDTITRTPFPLYFLDRASTIPVLLDGMNYEIVGHSNVPFPYVAI |
| | DQSTPEVNDTTWDTSFGVDISNRFFSMASSAAVTSTPFMGSLHGL |
| | ETYYNRTNQIIFGDPNVLPMCNLTKMLMIERAVATAARAALKYPY |
| | IYTSNAQNFTVNAQPNLTLVKLLMPYAFGEILNELGFFENTTTAA |
| | ALAAVQFNTTMLAAVRSAYMMDRVYDCIFSGNAYPFHVLTAAQQQ |
| | EVRWIIYSAIQNQLARCNQSIPYLGPDSNSSNSYNNMAPGCVPRI |
| | GINNLTEMLLPGLPYSNFNITVFIPESLYYNFGISRCLDGTDWTD |
| | VMGYLINATTRNNRKCNTGCIVGIAVASALVASFLVVAIVIMTSK |
| | RRRLATVVAPAATSEPKFISTLDMTSEEGSRNPLTR (SEQ ID |
| | NO: 59) |

Leishmania tropica

| | |
|---|---|
| LTRL590_100007000 | >LTRL590_100007000\|Leishmania tropica L590\| |
| | hypothetical protein, conserved\|protein\| |
| | length = 757 |
| | MGRCIRRVPAAAAAAALLLALVAAAAVSTTTARAYDHAGITVAGAI |
| | MVGQNLQGKAGASRILNPFAICANFDTADVEDTTLLIGGASYFFTL |
| | NRYSTYLGFWYGQGSMNLNSGPIDKVRLTGVFGCVTLRPNSSNGLV |
| | TSTVYYVQNDGMLYWVSNSVVYLTQVTHGISFVDVTVHDNNVYLLS |
| | TQNRIYRCSIGTGGAVVGSACTQITLTGSTEFDQLITVPSDFRGFA |
| | VSSCGIFIAPTSDLYWFSLSGVFITKSAGVTFVDIKLTSSSDTANT |
| | GTSVFMAASTSAVYAVTASSATISYALVSGKETKSCNPALNNVDSD |
| | TSPTFCGIARIYPLNTDMVYMTTGVASVVRAIIVSNTTISDTITRT |
| | PFPVYFLDNSSIIPLILDGMNYELVGNSNIPFPYVAINHSTPEVDD |
| | STWDTTFSVDVSNRFFSTVSSAAVVSTPFMGSLHGLQAYYNRTNQI |
| | LFGDPNVLPMCNLTKMQRIERAVAADARAALQYPYIYTSKAQNFTV |
| | NAHPNLTLLKLLMPYPFGEILNESGFFENTTTPAVLANVHFNTTML |
| | AAVRNAYTPDFVYDCIFAGNAFPFQILTAAQQQLVRWIIYTAIQEQ |
| | LAKCAENSPSYTGSDSSSSDSHDDMVPGCVPRVGIGNLTELVMPGM |
| | PYSNYNITVFIPEGLHYNFSISRCLDGTDWTNVTDYLQHATTPRTR |
| | KCGTGCIVSIAVVSAVVAAILVVAIVIATSKRRRLATVVAPAFTVE |
| | PKFASTLDMSSEEGSRNPLNR (SEQ ID NO: 60) |

According to a further aspect of the invention, there is provided a pharmaceutical composition comprising a trypanosomal vaccine as defined herein. Thus, such a pharmaceutical composition may also be referred to as a vaccine composition.

In one embodiment, the vaccine composition additionally comprises invariant flagellum antigen. In one embodiment, the invariant flagellum antigen comprises the amino acid sequence as set forth in SEQ ID NO: 61, or a protein having at least 90% sequence identity to said amino acid sequence, or a fragment of said amino acid sequence thereof, or a nucleic acid molecule encoding said protein.

The amino acid sequence of SEQ ID NO: 61 is an invariant flagellum antigen from T. vivax as detailed in WO 2020/144465, the contents of which are hereby incorporated by reference (in particular sequences, compositions and methods contained therein). Thus, in one embodiment the invariant flagellum antigen is from T. vivax.

References herein to the amino acid sequence set forth in SEQ ID NO: 61 refer to:

(SEQ ID NO: 61)
MRCHEPPTPPQLSATCCVAEEIDTYNKHLDALMQIIGDAIKNISTNEDNA
RARAEGLKGCNLHYVQFAVAHTEGSVVAARREAVKAQNTIKGSTSLLKKV
TIDISNSFRNISSKCNELREKYPSLIPADKNSPPNITFKKAVQLYVKNFS
TCNVMYAKKLLRLVAQSEKIEAEVSRAVERTNASTMELAKLDKVAVQLNK
DITSNRTWAGCKLAEYHGQMNFVFMGFYVLLSDILDELHSLLKKSKSMQP
TRLTQEEVRRALSKAEQVCHDVSRFVKSLGSTLRDFTNFVHRLRKEYLHG
ILRNASGFRESFERCYKVATNNSVTRLESTVEEITANNENIAAWESMTVH
QWKDVSKKLRQSLLTVLGGSNEYILLYGYFQEFDSMSVREFSNTVRAFRQ
SITEMSVARNVVGVAAKTVAADRKRILCRSVLMFNKGTAGSESARKLYEL
CKTRMPVEEPDSSREDGVVGTSGSEEEISGKDGGTSFSVSDADYWEWDVP
PKVLEESSGDLLYDTAVDLHTKRKSPFYQVGS.

The amino acid sequence of SEQ ID NO: 61 corresponds to the ectodomain of a cell surface T. vivax protein known as TvY486_0807240.

The full length amino acid sequence of TvY486_0807240 is shown below:

(SEQ ID NO: 62)
MEVMLFDYFHVLPISCKPRNFCIAFMLMFLRFCPVFAMRCHEPPTPPQLS
ATCCVAEEIDTYNKHLDALMQIIGDAIKNISTNEDNARARAEGLKGCNLH
YVQFAVAHTEGSVVAARREAVKAQNTIKGSTSLLKKVTIDISNSFRNISS
KCNELREKYPSLIPADKNSPPNITFKKAVQLYVKNFSTCNVMYAKKLLRL
VAQSEKIEAEVSRAVERTNASTMELAKLDKVAVQLNKDITSNRTWAGCKL
AEYHGQMNFVFMGFYVLLSDILDELHSLLKKSKSMQPTRLTQEEVRRALS
KAEQVCHDVSRFVKSLGSTLRDFTNFVHRLRKEYLHGILRNASGFRESFE
RCYKVATNNSVTRLESTVEEITANNENIAAWESMTVHQWKDVSKKLRQSL
LTVLGGSNEYILLYGYFQEFDSMSVREFSNTVRAFRQSITEMSVARNVVG
VAAKTVAADRKRILCRSVLMFNKGTAGSESARKLYELCKTRMPVEEPDSS
REDGVVGTSGSEEEISGKDGGTSESVSDADYWEWDVPPKVLEESSGDLLY
DTAVDLHTKRKSPFYQVGSIAFGVFLLVVSCGVGILMFVRRWYAACVARS
ADGGTDC.

The underlined portion represents the ectodomain region of TvY486_0807240.

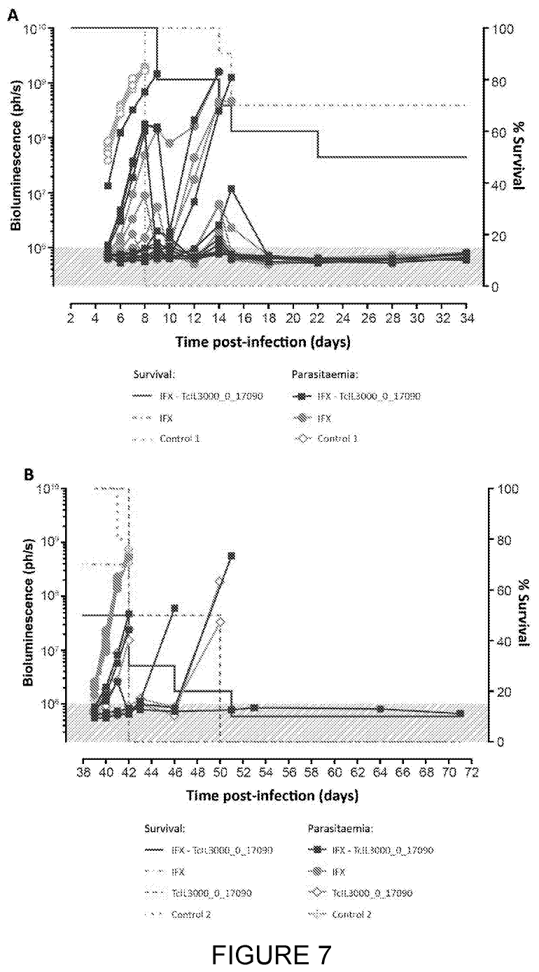

TvY486_0807240 is also referred to herein as either V23 or IFX (invariant flagellum antigen from T. vivax). Data is presented herein which surprisingly shows that IFX together with TcIL3000_0_35140 or TcIL3000_0_17090 elicited protection in vaccinated animals to both T. congolense and T. vivax (see Example 4 and FIG. 7). This, coupled with the remaining data presented herein, indicates that a vaccine composition comprising TcIL3000_0_35140 or TcIL3000_0_17090 together with IFX represents a good candidate for trypanosomal infection, such as animal African trypanosomiasis (AAT) caused by T. congolense and/or T. vivax. Thus, a composition comprising TcIL3000_0_35140 or TcIL3000_0_17090 together with IFX offers the possibility of vaccinating animals to provide protection against both T. congolense and T. vivax.

In another embodiment, the vaccine composition comprises a protein which consists of the amino acid sequence as set forth in SEQ ID NO: 61.

In a further embodiment, the vaccine composition additionally comprises one or more adjuvants. References herein to the term “adjuvant” refer to a compound that, when used in combination with a specific immunogen in a formulation, will augment or otherwise alter or modify the resultant immune response. Modification of the immune response can include intensification or broadening the specificity of either or both antibody and cellular immune responses. Modification of the immune response can also mean decreasing or suppressing certain antigen-specific immune responses.

In one embodiment, at least about 1 ng and up to about 50 ng adjuvant is present within the vaccine composition. In a further embodiment, at least about 1 μg and up to about 20 μg adjuvant is present within the vaccine composition. Examples of suitable adjuvants include: alum; aluminum hydroxide; aluminum phosphate; calcium phosphate hydroxide; paraffin oil; killed bacteria such as Bordetella pertussis, Mycobacterium bovis and toxoids; squalene, detergents; plant saponins from quillaja, soybean, polygala senega; cytokines such as IL-1, IL-2, IL-12; Freund's complete adjuvant; and Freund's incomplete adjuvant. One further example of a suitable adjuvant includes TiterMax® Gold Adjuvant (Sigma-Aldrich) which contains three essential ingredients: a block copolymer, CRL-8300, squalene (a metabolizable oil) and a sorbitan monooleate.

In a yet further embodiment, said adjuvant comprises aluminium hydroxide, such as a wet gel suspension of aluminium hydroxide, in particular Alhydrogel®, more particularly Alhydrogel® 2%. In one particular embodiment, said adjuvant comprises Montanide® ISA 201 VG. This adjuvant is a water-in-oil-in-water adjuvant and full details of this adjuvant may be found: https://www.seppic.com/montanide-isa-w-o-w. In an alternative embodiment, said adjuvant comprises Quil-A®. Quil-A® adjuvant is a saponin adjuvant which is used in a wide variety of veterinary vaccines. Full details of Quil-A® may be found: https://www.invivogen.com/quila.

In one embodiment, the vaccine composition additionally comprises a pharmaceutically acceptable carrier, diluent, excipient, or combination thereof, in which the immunogen (i.e. the proteins as defined herein) is/are suspended or dissolved.

Pharmaceutically acceptable carriers are known, and include but are not limited to, water for injection, saline solution, buffered saline, dextrose, water, glycerol, sterile isotonic aqueous buffer, and combinations thereof. For parenteral administration, such as subcutaneous injection, the carrier may include water, saline, alcohol, a fat, a wax, a buffer or combinations thereof. Pharmaceutically acceptable carriers, diluents, and other excipients are described in detail in Remington's Pharmaceutical Sciences (Mack Pub. Co. N.J. current edition). The formulation should suit the mode of administration. In a preferred embodiment, the formulation is suitable for administration to humans, preferably is sterile, non-particulate and/or non-pyrogenic.

In other embodiments, the vaccine composition can include one or more diluents, preservatives, solubilizers and/or emulsifiers. For example, the vaccine composition can include minor amounts of wetting or emulsifying agents, or pH buffering agents to improve vaccine efficacy. The composition can be a solid form, such as a lyophilized powder suitable for reconstitution, a liquid solution, suspension, emulsion, tablet, pill, capsule, sustained release formulation, or powder. Oral formulation can include standard carriers such as pharmaceutical grades of mannitol, lactose, starch, magnesium stearate, sodium saccharine, cellulose, magnesium carbonate, etc.

It may also be desirable to include other components in a vaccine composition, such as delivery vehicles including but not limited to aluminum salts, water-in-oil emulsions, biodegradable oil vehicles, oil-in-water emulsions, biodegradable microcapsules, and liposomes. In other embodiments, the vaccine composition can include antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

Administration of the vaccine composition can be systemic or local. Methods of administering a vaccine composition include, but are not limited to, parenteral administration (e.g., intradermal, intramuscular, intravenous and subcutaneous), epidural, and mucosal (e.g., intranasal and oral or pulmonary routes or by suppositories). In a specific embodiment, compositions described herein are administered intramuscularly, intravenously, subcutaneously, transdermally or intradermally. The compositions may be administered by any convenient route, for example by infusion or bolus injection, by absorption through epithelial or mucocutaneous linings (e.g., oral mucous, colon, conjunctiva, nasopharynx, oropharynx, vagina, urethra, urinary bladder and intestinal mucosa, etc.) and may be administered together with other biologically active agents. In some embodiments, intranasal or other mucosal routes of administration of a composition may induce an antibody or other immune response that is substantially higher than other routes of administration. In another embodiment, intranasal or other mucosal routes of administration of a composition described herein may induce an antibody or other immune response at the site of immunization.

In one embodiment, the vaccine composition has a volume of between about 50 μl and about 10 ml, such as 1 ml.

According to a further aspect of the invention, there is provided a method of preventing or treating trypanosomal infection in a mammal which comprises administering to the mammal a therapeutically effective amount of the vaccine composition as defined herein.

References herein to “trypanosomal infection” refer to infection by a trypanosome as defined herein, in particular T. congolense. Thus, in one embodiment, the trypanosomal infection is an infection mediated by Trypanosoma congolense. In another embodiment, the the trypanosomal infection is an infection mediated by Trypanosoma vivax.

In one embodiment, the trypanosomal infection is animal African trypanosomiasis (AAT).

References herein to “effective amount” refer to a dose which is sufficient or most likely to elicit antibodies such that the immunized subject has reduced severity of infection.

According to a further aspect of the invention, there is provided a method of inducing an immune response in a mammal, wherein the method includes administering to the mammal, an effective amount of the vaccine composition as defined herein.

Examples of suitable mammals include ungulates, such as those selected from humans, cattle, goats, sheep, horses, pigs, dogs and camels.

In one embodiment, the vaccine composition is administered in a single dose regimen. In another embodiment, the vaccine composition is administered in a two dose regimen that includes a first and a second dose. In one embodiment, the second dose is administered at least about 1 week, 2 weeks, 3 weeks, 1 month or 1 year after the first dose. In another embodiment, the vaccine composition is administered in a three dose regimen.

According to a further aspect of the invention, there is provided a kit of parts comprising a vaccine composition as defined herein, a medical instrument or other means for administering the vaccine composition and instructions for use.

In one embodiment, the vaccine composition is packaged in a hermetically sealed container such as an ampoule or sachette indicating the quantity of composition. In one embodiment, the composition is supplied as a liquid. In another embodiment, the composition is supplied as a dry sterilized lyophilized powder or water free concentrate in a hermetically sealed container, wherein the composition can be reconstituted, for example, with water or saline, to obtain an appropriate concentration for administration to a subject.

When the vaccine composition is systemically administered, for example, by subcutaneous or intramuscular injection, a needle and syringe, or a needle-less injection device can be used. The vaccine formulation can be enclosed in ampoules, disposable syringes or multiple dose vials made of glass or plastic.

The invention is further described below with reference to the following examples.

### Example 1—Materials and Methods

Design, Synthesis and Purification of T. congolense TcIL3000_0_17090 and TcIL3000_0_35140

The regions corresponding to the entire extracellular domains of TcIL3000_0_17090 and TcIL3000_0_35140 were determined by using transmembrane (TMHMMv2.0 (Sonnhammer et al., (1998) Proceedings International Conference on Intelligent Systems for Molecular Biology 6, 175-182) and signal peptide prediction software (SignalP v4.0 (Petersen et al., (2011) Nature methods 8, 785-786). Sequences encoding the entire extracellular domains of these proteins (see sequences appendix) from the IL3000 strain of Trypanosoma congolense, with the exception of their signal peptide, were made by gene synthesis (Twist Biosciences, USA). All sequences were codon-optimized for expression in human cells. The coding sequences were flanked by unique NotI and AscI sites and cloned into a derivative of the pTT3 expression vector between the leader sequence of the mouse variable light chain 7-33 (Crosnier et al., (2013) Molecular & cellular proteomics: MCP 12, 3976-3986). The ectodomains were expressed as a soluble recombinant protein in HEK293 cells as described (Crosnier et al., 2013, supra). Protein was purified by Ni2+ immobilised metal ion affinity chromatography using HisTRAP columns (GEHealthcare, UK), eluted in 400 mM imidazole as described (Bartholdson et al., (2012) PLoS pathogens 8, e1003031), dialysed into HBS, aliquoted and snap-frozen prior to immunisation.

Animals, Immunisations, Challenge and Bioluminescence Measurement

All animal experiments were performed in accordance with UK Home office legislation and according to local ethical review board approval. Six to eight-week old female BALB/c mice were bred and housed at the Research Support Facility of the Wellcome Trust Sanger Institute. Recombinant proteins were adjuvanted in QuilA and animals immunised subcutaneously with an initial prime followed by two further booster immunisations given at two week intervals.

Vaccinated animals were rested for 4 weeks after the final immunisation to mitigate any possible non-specific protective effects elicited by residual adjuvant. Animal challenges were performed using a transgenic form of the T. congolense IL3000 strain genetically engineered to ubiquitously express the firefly luciferase enzyme. Parasites were maintained by weekly passage in wild type BALB/c mice. For infection challenges, bloodstream forms of T. congolense parasites were obtained from the blood of an infected donor mouse at the peak of parasitaemia and between 100 to 1000 parasites were used to infect mice by intravenous injection.

From day three post-infection, animals were injected intraperitoneally with luciferase substrate, D-luciferin (D-Luciferin potassium salt, Source BioScience, Nottingham, UK) at a dose of 200 mg/kg, 10 minutes before bioluminescence acquisitions. The mice were anaesthetized with 3% isoflurane and placed in the imaging chamber for analysis. Emitted photons were acquired by a charge coupled device (CCD) camera (IVIS Spectrum Imaging System, Perkin Elmer). Total photons emitted from the image of each mouse were quantified using Living Image software (Xenogen Corporation, Almeda, California), and results were expressed as number of photons/sec/ROI.

Immune sera was elicited by subcutaneously immunising a cohort of female BALB/c mice with the purified ectodomain of TcIL3000_0_17090 using QuilA as an adjuvant with a prime followed by two booster immunisations separated by two week intervals. Immune sera were collected from immunised mice by cardiac puncture, aliquoted and stored frozen until use. Control sera were taken from unimmunised mice. Immune and control sera were passively transferred to groups of recipient female BALB/c mice by intravenous injection on the day before, on the day, and the day after inoculation with the transgenic T. congolense parasite. Parasitaemia was quantified by bioluminescent imaging using an IVIS instrument.

### Example 2

To discover potential subunit vaccine candidates for T. congolense, the genome sequence was analysed to identify proteins that fulfilled the following criteria: 1) were predicted to encode cell surface proteins that would be accessible to vaccine-elicited host antibodies; 2) did not belong to a paralogous group of parasite proteins that might indicate functional redundancy; 3) contained more than 300 amino acids and so are likely to project beyond the VSG coat on the parasite membrane. Two protein that met these criteria were the related proteins known by their accession numbers TcIL3000_0_35140 and TcIL3000_0_17090.

### Results

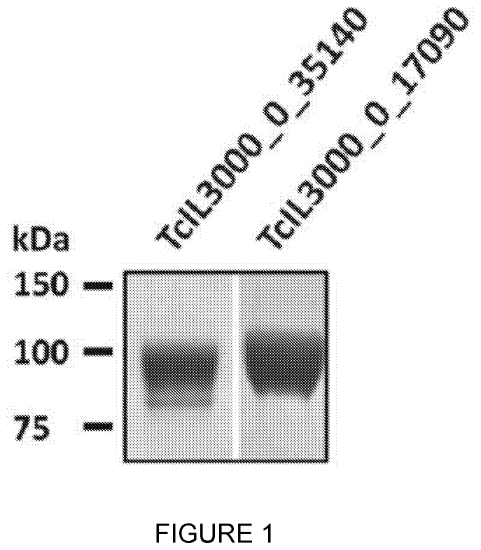

To increase the chances that the extracellular regions of the protein were expressed in a correctly folded conformation and therefore elicit antibodies that would bind to the native parasite protein, we expressed both these proteins using a mammalian expression system to promote the formation of structurally-critical disulphide bonds. The entire ectodomain region was identified and the genes constructed by gene synthesis using codons optimised for expression in human cells. These gene constructs were cloned into a mammalian protein expression plasmid. Human embryonic kidney (HEK)293 cells were transfected with these plasmids and the proteins secreted into the tissue culture medium. The proteins were purified from the tissue culture supernatant by immobilised metal ion chromatography (IMAC) and resolved as a series of glycoforms by SDS-PAGE (FIG. 1).

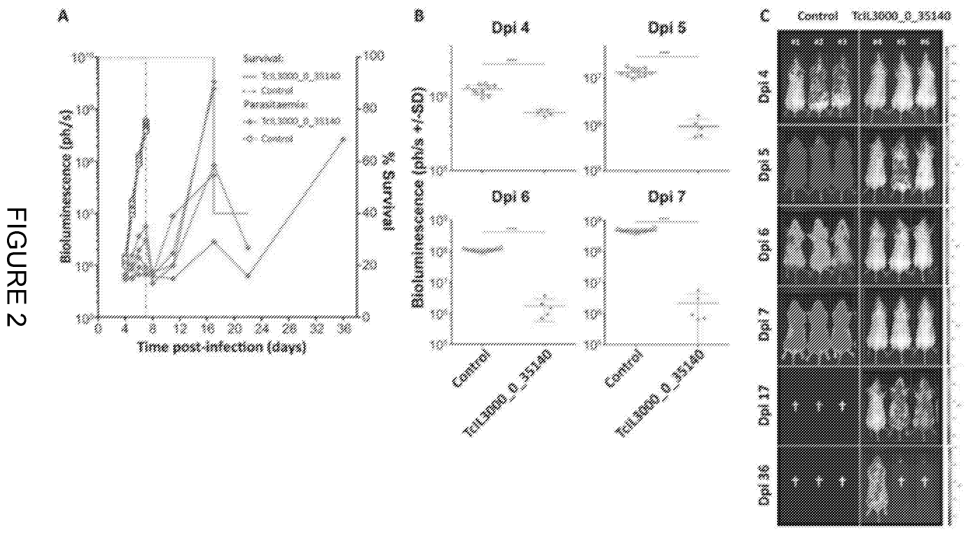

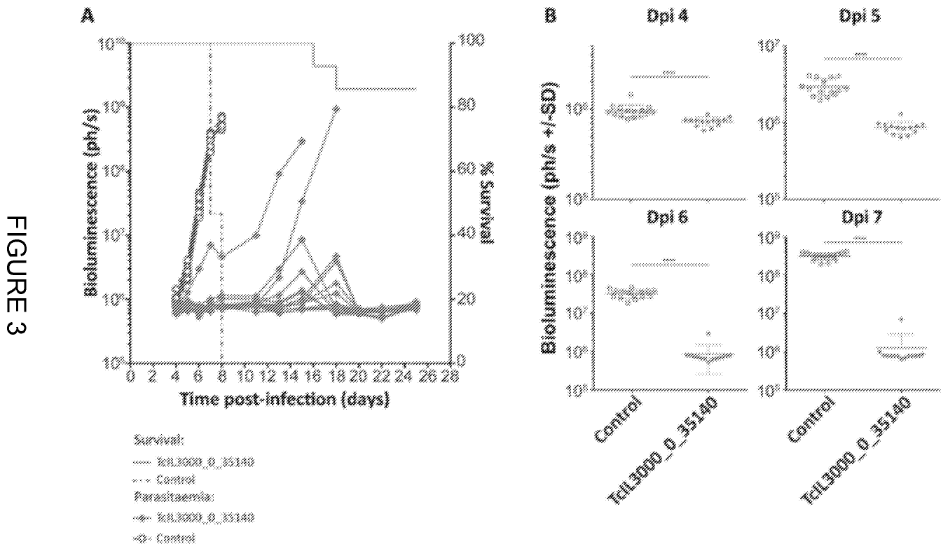

Groups of five female BALB/c mice were immunised subcutaneously with the purified ectodomain of TcIL3000_0_35140 using a prime followed by two boost regime with the protein adjuvanted with QuilA; control animals were immunised with adjuvant only. Vaccinated animals were challenged with T. congolense parasites delivered intravenously from the blood of an infected donor animal. Animals immunised with TcIL3000_0_35140 were protected from infection relative to adjuvant-only control mice over the first seven days of infection (FIG. 2A, B, C). Two of the five mice immunised with the ectodomain of TcIL3000_0_35140 survived the infection challenge beyond day 20. To confirm these results, a larger cohort of 15 mice were immunised with an independent preparation of the TcIL3000_0_35140 ectodomain and again all vaccinated animals were protected up to day 9, a time at which all adjuvant-only controls were removed from the study (FIG. 3A, B). Thirteen of the fifteen (87%) vaccinated animals showed no evidence of parasitaemia at 25 days post infection (FIG. 3A).

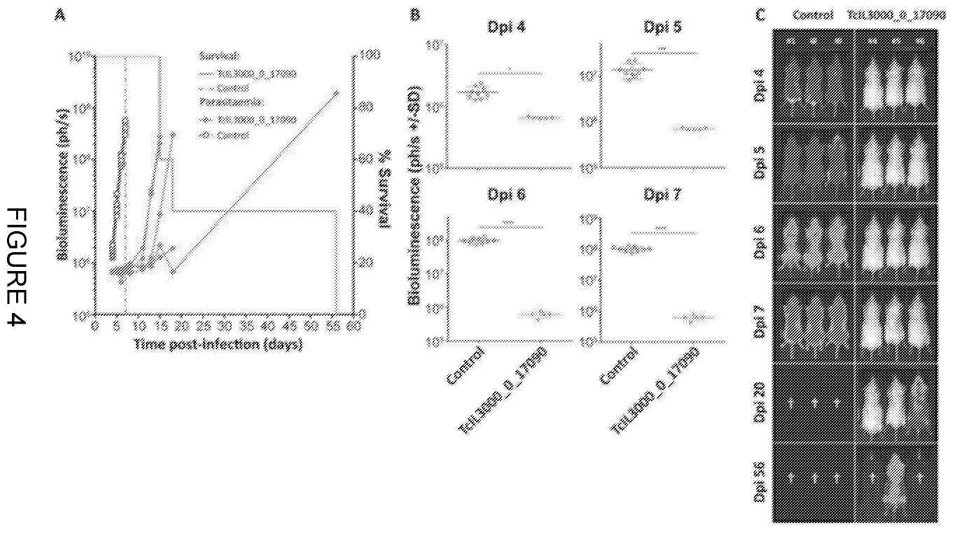

To further confirm these results, a group of five mice were vaccinated with a different but related protein encoded in the genome of T. congolenese called TcIL3000_0_17090. TcIL3000_0_35140 and TcIL3000_0_17090 are almost identical in their predicted extracellular region, sharing greater than 98% amino acid identity in their sequence. We again used a protein-in-adjuvant formulation using a prime and two boosts of the protein adjuvanted in QuilA and a control group of five mice receiving adjuvant alone. Again, we observed robust protection of the mice with all vaccinated animals surviving beyond day 10, a time at which all control animals had to be removed from the study (FIG. 4A, B, C).

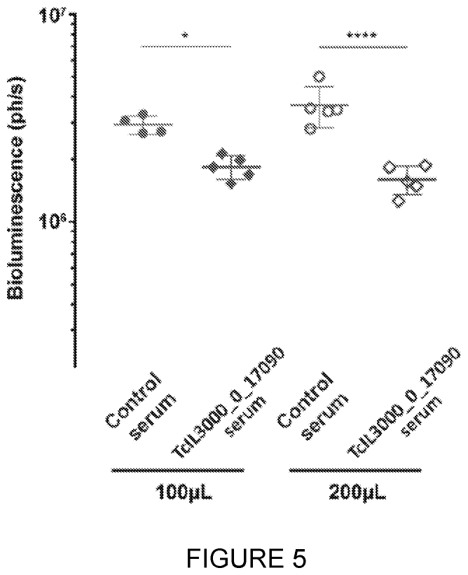

To begin to determine the immunological mechanisms of protection and further validate the protective effects of vaccination, we next asked whether animals could be passively protected from infection by the transfer of immune serum from vaccinated animals. To obtain immune sera, a cohort of animals were vaccinated with the purified extracellular region of TcIL3000_0_17090 and the immune sera collected; non-immune sera were obtained from unimmunised animals. Animals were dosed with the immune sera by delivering either 100 or 200 microlitres of sera intravenously on three consecutive days and challenged with T. congolense parasite. Those animals receiving immune sera showed reduced levels of parasitameia compared to controls, and showed evidence of a dose-dependent effect (FIG. 5).

### Discussion

Animal African trypanosomiasis continues to be a significant impediment in the successful raising of livestock animals in sub-Saharan Africa and previous attempts to vaccinate against the trypanosome parasites that cause this disease have been unsuccessful. Here we have shown that vaccinating animals with a recombinant protein comprising the entire ectodomain of either TcIL3000_0_17090 or TcIL3000_0_35140 T. congolense cell surface proteins confers protection in a mouse model of infection demonstrating that either protein could be an effective subunit vaccine. We note that the disease is acute in the BALB/c mice used in our infection trials since control mice develop rapid uncontrolled parasitaemia whereas in livestock animals such as goats and cattle the infection is typically a chronic disease with lower parasitaemia suggesting the mouse infection model provides a stringent test of these vaccine candidates. We envisage that a vaccine containing either TcIL3000_0_17090 or TcIL3000_0_35140 in whole or in part and in the context of an appropriate adjuvant will constitute a vaccine to treat this disease in livestock animals.

### Example 3

The species of parasite known as T. congolense is composed of three recognised strains known as “Savannah”, “Forest” and “Kilifi”. The Savannah strain is generally recognised as the most prevalent and the IL3000 isolate used in the above vaccine screens belongs to this strain. Parasite vaccines, however, are known to show strain-specific protective effects and so to show that the TcIL3000_0_35140 and TcIL3000_0_17090 vaccine candidates are able to elicit strain-transcending immunity, mice vaccinated with the TcIL3000_0_35140 protein were challenged with a strain known as DIN80 which is a “Forest-type” strain.

### Results & Discussion

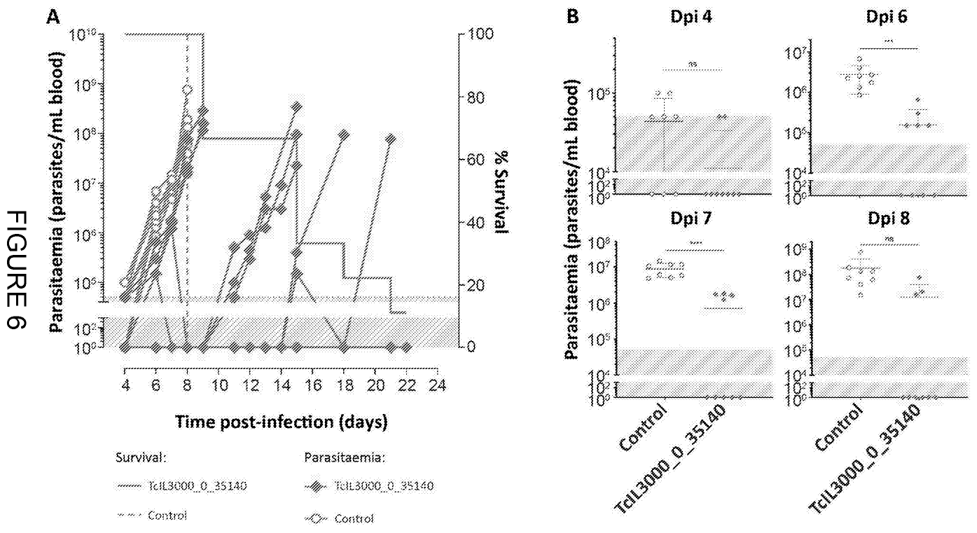

Mice vaccinated using TcIL3000_0_35140 and TcIL3000_0_17090 were able to control infection of the “Forest-type” DIN80 strain when compared to controls with one out of nine animals being sterilely protected (FIG. 6A, B). Together, these data demonstrate that the TcIL3000_0_35140 and TcIL3000_0_17090 subunit vaccines are able to elicit cross-protection to different strains of T. congolense.

### Example 4

While T. congolense is a major etiological agent of animal African trypanosomiasis, another species of trypanosome that is genetically very distinct called T. vivax, can also cause this disease. While the geographic distributions of these parasites differ, there is a need to vaccinate livestock animals against both T. congolense and T. vivax. Earlier research by the inventors has already identified a subunit vaccine candidate for T. vivax called invariant flagellum antigen from T. vivax or “IFX” which offers the possibility of vaccinating animals with both proteins to protect both T. congolense and T. vivax.

### Results & Discussion

Three groups of animals were therefore vaccinated with either IFX alone, TcIL3000_0_17090 alone or with both proteins using a co-administration procedure. The animals that had been vaccinated with either IFX alone or the IFX-TcIL3000_0_17090 combination were first challenged with T. vivax (FIG. 7A). We observed that the IFX-vaccinated animals were protected against T. vivax infection as expected, with over half of the animals showing sterile protection. The mice that were vaccinated with both IFX and TcIL3000_0_17090 were also able to control the infection with five of the ten animals showing sterile protection (FIG. 7A). After waiting 35 days after the challenge with T. vivax to ensure there was no recrudescence, we next challenged all three groups with T. congolense. As expected, the animals vaccinated with IFX showed no protection to T. congolense, and those animals vaccinated with TcIL3000_0_17090 alone were able to control a T. congolense infection (FIG. 7B). The animals vaccinated with both IFX and TcIL3000_0_17090 and which had already survived the T. vivax challenge were also able to control the T. congolense infection, with one animal sterilely protected against challenge with both parasite species (FIG. 7B). Together, these data provide evidence that vaccinating animals with both IFX and TcIL3000_0_35140 or TcIL3000_0_17090 can elicit protection to both T. congolense and T. vivax.

1. A trypanosomal vaccine, comprising: a flagellum adhesion protein 1 (FLA1) binding protein.

2. The trypanosomal vaccine of claim 1, wherein the FLA1 binding protein comprises a sequence selected from an amino acid sequence as set forth in SEQ ID NO: 1, a sequence having at least 90% sequence identity to SEQ ID NO: 1, a fragment of SEQ ID NO: 1, and a nucleic acid sequence encoding said protein.

3. The trypanosomal vaccine of claim 2, comprising a protein which consists of the amino acid sequence as set forth in SEQ ID NO: 1.

4. The trypanosomal vaccine of claim 1, wherein the FLA1 binding protein comprises a sequence selected from an amino acid sequence as set forth in SEQ ID NO: 3, a sequence having at least 90% sequence identity to SEO ID NO: 3, a fragment of SEQ ID NO: 3, and a nucleic acid sequence encoding said protein.

5. The trypanosomal vaccine of claim 4, comprising a protein which consists of the amino acid sequence as set forth in SEQ ID NO: 3.

6. The trypanosomal vaccine of claim 1, which is a Trypanosoma congolense vaccine.

7. The trypanosomal vaccine of claim 1, which is a Trypanosoma vaccine and wherein the FLA1 binding protein is selected from a protein of any one of SEQ ID NOs: 5 to 44.

8. The trypanosomal vaccine of claim 1, which is a Leishmania vaccine and wherein the FLA1 binding protein is selected from a protein of any one of SEQ ID NOs: 45 to 60.

9. A pharmaceutical composition comprising a trypanosomal vaccine of claim 1.

10. The pharmaceutical composition of claim 9, which additionally comprises invariant flagellum antigen.

11. The pharmaceutical composition of claim 10, wherein the invariant flagellum antigen comprises a sequence selected from an amino acid sequence as set forth in SEQ ID NO: 61, a sequence having at least 90% sequence identity to SEQ ID NO: 61, a fragment of SEQ ID NO: 61, and a nucleic acid sequence encoding said protein.

12. The pharmaceutical composition of claim 11, comprising a protein which consists of the amino acid sequence as set forth in SEQ ID NO: 61.

13. The pharmaceutical composition of claim 9, which additionally comprises one or more adjuvants.

14. The pharmaceutical composition of claim 13, wherein said adjuvant comprises a saponin adjuvant.

15. The pharmaceutical composition of claim 9, which additionally comprises a pharmaceutically acceptable carrier, diluent, excipient, or combination thereof.

16. The pharmaceutical composition claim 9, which is adapted for parenteral administration, epidural administration, or mucosal administration.

17. A method of preventing or treating trypanosomal infection in a mammal, comprising: administering to the mammal a therapeutically effective amount of the vaccine composition of claim 9.

18. The method of claim 17, wherein the trypanosomal infection is animal African trypanosomiasis (AAT) or is an infection mediated by Trypanosoma congolense or by Trypanosoma vivax.

19. (canceled)

20. (canceled)

21. A method of inducing an immune response in a mammal, comprising: administering to the mammal, an effective amount of the vaccine composition of claim 9.

22. (canceled)

23. A kit of parts, comprising a vaccine composition of claim 9, a medical instrument or other means for administering the vaccine composition and instructions for use.


SEQUENCE LISTING (62 sequences)

SEQ ID NO: 1 - protein, 641 aa, Trypanosoma congolense
AVAHVKRNRRVETVAGAYGLTGMVDGVPPDSRLSSPMAICRGRTADEILVGTASGLRTYSRSSGELGTLFTSSVKVVGGSTGGSAYGNPRSCVHRGIDNSSVIYFVDGQKDVKYYKNNEVLSKDTVANASLTAMTIFGGSLYMTDQINKALVTCKLSADGAPHDCLSKKKLNDTCGFNTFTGITSTAKGIFIAGQGSTTPGNICWIGLDDTTVTKLGQGEYVDVSSTSSGDLYAVSKTQIFHLEPAGSAPQLKTVAGVKGTPCLPTPDGEDIRFCELNKILAIADHELYVTSERSHLLRAVILPPVRVQAVFSGRPVPVGYPEGDTLDWIVENLVKDVNEALQTTESLIDPSTVYVDPDTWTTRFVALVQQSDFDDAATERALGEGNYTYITAALDEYYNETDQAVYMDSVMVPYCSEAALDAIRRRIAEEARRVLDFPLIYADMPVELEGSGVENVTMVKLLMPASFNNETVSELLEAADLTGFAHSAIKEMRGGETRVSVVLPNPPFNFSGVTPDVDQDIRWYVHGNVMKQLDICEKLNAKGAAPAPEPVEDGNESGGGVVYTGEFCQSSITNRTETQNLKPPYDQKNTYEIFLPNKYDFNASWCVDIVDWRELNDWLSNVTAGSHIEDASWCGQGCII

SEQ ID NO: 2 - protein, 711 aa, Trypanosoma congolense
MRTGRALQVLLHATIISLGLVECAVAHVKRNRRVETVAGAYGLTGMVDGVPPDSRLSSPMAICRGRTADEILVGTASGLRTYSRSSGELGTLFTSSVKVVGGSTGGSAYGNPRSCVHRGIDNSSVIYFVDGQKDVKYYKNNEVLSKDTVANASLTAMTIFGGSLYMTDQINKALVTCKLSADGAPHDCLSKKKLNDTCGFNTFTGITSTAKGIFIAGQGSTTPGNICWIGLDDTTVTKLGQGEYVDVSSTSSGDLYAVSKTQIFHLEPAGSAPQLKTVAGVKGTPCLPTPDGEDIRFCELNKILAIADHELYVTSERSHLLRAVILPPVRVQAVFSGRPVPVGYPEGDTLDWIVENLVKDVNEALQTTESLIDPSTVYVDPDTWTTRFVALVQQSDFDDAATERALGEGNYTYITAALDEYYNETDQAVYMDSVMVPYCSEAALDAIRRRIAEEARRVLDFPLIYADMPVELEGSGVENVTMVKLLMPASFNNETVSELLEAADLTGFAHSAIKEMRGGETRVSVVLPNPPFNFSGVTPDVDQDIRWYVHGNVMKQLDICEKLNAKGAAPAPEPVEDGNESGGGVVYTGEFCQSSITNRTETQNLKPPYDQKNTYEIFLPNKYDFNASWCVDIVDWRELNDWLSNVTAGSHIEDASWCGQGCIIALAVVGALLTTGLVVVAVVLTSKRRRLAAVVAPPRPKFVSATEDEED

SEQ ID NO: 3 - protein, 637 aa, Trypanosoma congolense
HVKRNRRVETVAGAYGLTGMVDGVPPDSRLSSPMAICRGRTADEILVGTASGLRTYSRSSGELGTLFTSSVKVVGGSTGGSAYGNPRSCVHRGIDNSSVIYFVDGQKDVKYYKNNEVLSKDTVANASLTAMTIFGGSLYMTDQINKALVTCKLSADGAPHDCLSKKKLNDTCGFNTFTGITSTAKGIFIAGQGSTTPGNICWIGLDDTTVTKLGQGEYVDVSSTSSGDLYAVSKTQIFHLEPAGSAPQLKTVAGVKGTPCLPTPDGEDIRFCELNKILAIADHELYVTSERSHLLRAVILPPVRVQAVFSGRPVPVGYPEGDTLDWIVENLVKDVNEALQTTESLIDPSTVYVDPDTWTTRFVALVQQSDFDDAATERALGEGNYTYITAALDEYYNETDQAVYMDSVMVPYCSEAALDAIRRKIAEEARRVLDFPLIYADMPVELEGSGAENVTMVKLLMPASFNNETVSELLEAADLTGFAHSAIKEMRGGETRVSVVLPNPPFNFSGVTPDVDQDIRWYVHGNVMKQLDICEKLNAKGAAPAPEPVEDSNESGGGVVYTGEFCQSSITNRTETQNLKPPYDQKNTYEIFLPNKYDFNASWCVDIVDWRELNDWLSNVTVGSHIEDASWCGQGCI

SEQ ID NO: 4 - protein, 711 aa, Trypanosoma congolense
MRTGRALQLLLHATIIFLGLVECAVAHVKRNRRVETVAGAYGLTGMVDGVPPDSRLSSPMAICRGRTADEILVGTASGLRTYSRSSGELGTLFTSSVKVVGGSTGGSAYGNPRSCVHRGIDNSSVIYFVDGQKDVKYYKNNEVLSKDTVANASLTAMTIFGGSLYMTDQINKALVTCKLSADGAPHDCLSKKKLNDTCGFNTFTGITSTAKGIFIAGQGSTTPGNICWIGLDDTTVTKLGQGEYVDVSSTSSGDLYAVSKTQIFHLEPAGSAPQLKTVAGVKGTPCLPTPDGEDIRFCELNKILAIADHELYVTSERSHLLRAVILPPVRVQAVFSGRPVPVGYPEGDTLDWIVENLVKDVNEALQTTESLIDPSTVYVDPDTWTTRFVALVQQSDFDDAATERALGEGNYTYITAALDEYYNETDQAVYMDSVMVPYCSEAALDAIRRKIAEEARRVLDFPLIYADMPVELEGSGAENVTMVKLLMPASFNNETVSELLEAADLTGFAHSAIKEMRGGETRVSVVLPNPPFNFSGVTPDVDQDIRWYVHGNVMKQLDICEKLNAKGAAPAPEPVEDSNESGGGVVYTGEFCQSSITNRTETQNLKPPYDQKNTYEIFLPNKYDFNASWCVDIVDWRELNDWLSNVTVGSHIEDASWCGQGCIIALAVVGALLTTGLVVVAVVLTSKRRRLAAVVAPPRPKFVSATEDEED

SEQ ID NO: 5 - protein, 818 aa, Trypanosoma brucei
  misc_feature (108)..(108): Xaa can be any naturally occurring amino acid
  misc_feature (167)..(167): Xaa can be any naturally occurring amino acid
  misc_feature (403)..(403): Xaa can be any naturally occurring amino acid
MCFIFGVEMSNLAKRPMSLRKLPQLFLLIMIGIAFVAVECIGTPVKLPRRVDTVAGQFGVEGETNGYPNTTRLTEPYALCRGRTNDEILVGSSNSFRNYSRKTKETGXYLRYNVGDSVISGSSTINKPRSCVRRGSGNHTIIYFVDDQKDIKYIVGDDVSSFSVPTXGSLNAVAVHEGSLYVTDQNNKSVWKCGLGGAGKPQSCEEKKFTSVTLDAKPEGIAVTSKGIFVTARDSSNKGALLWLDMNGSNRKGNVSGGFVDVFSTESGMLYAATEKELYTVTATDTSLSVTLFAGKNTSSCYFPTNGEDIVLCDNSRLLVIEEYEMYVTSRAKHTMRALTLPPVNLTAIFRGRPAPVGYPNTTIMEQFVASLTEDVNKALGTNDSYVDPDSVRVDPDTWETNXTVFVQQTRFDNTTEEKLRSLTYTQTDKTVDEYYGLTDEYVYIDTVLVPFCDDASLVTIQRALAREAGRALNFSLVYADKPITFGSDVAENVTAVKLLMPHSFKNATTPKQLSAANLTDFAHNLVKDLRASDTRVDITFPDPPFNFSAVVPEREQEVRWFVHGKVMKQLEICERLGSQGDAAVIAAAADATARGKANVTLNTSGVKANDTGVGPNTTNTAGGANTTANVATNGTANVIVNPSTNATPTGTSNASVTNTTERAVPVVAPTQPSNGYAECRSAITNRTETQNMEPPYDRKHRYEVFLPKKYDFNVSWCVDIIDWRDLDEMLNNRTDEVVEKSLSWCGHGCIIAFAVVGSLIAACLVVLAVVLTSKRRRLAAVVAPPRPKFVSTVEDDEEDRVSNIGVPLTDGKGTTAP

SEQ ID NO: 6 - protein, 818 aa, Trypanosoma brucei
  misc_feature (287)..(287): Xaa can be any naturally occurring amino acid
  misc_feature (331)..(331): Xaa can be any naturally occurring amino acid
  misc_feature (403)..(403): Xaa can be any naturally occurring amino acid
MCFIFGVEMSNLAKRPMSLRKLPQLFLLIMIGIAFVAVECIGAPVKLPRRVDTVAGQFGFDGTTDGSSNVSMLSSPYALCRGRTNDEILVGSSNSFRNYSRKTKETGTFLRGGPTGGLVSADAKISKPRSCVRRGSGNHTIIYFVDDQNGLKYINDNEIQHVTVGDGLSLTSVAIYEKDLYVTDQNNKSVWRCNVGGAGKPQNCEEKKFTGLTFTAKPEGIAVTSKGIFVAARDSSNKGALLWLDMNGGGSKGNVSGGFVDVFSTESGMLYAATEKELYTVTATDSXFSVTLFAGKNTSSCYSHANGEDIVLCDNSRLLVIEEYEMYVTSXEKHTMRALTLPPVNLTAIFRGRPAPVGYPNTTIMEQFVASLTEDVNKALGTNDSYVDPDSVRVDPDTWETNXTVFVQQTRFDNTTEEKLRSLTYTQTDKTVDEYYGLTDEYVYIDTVLVPFCDDASLVTIQRALAREAGRALNFSLIYADKPITFGSDVAENVTAVKLLMPHSFKNATTPKQLSAANLTDFAHNLVKDLRASDTRVDITFPDPPFNFSAVVPEREQEVRWFVHGKVMKQLEICERLGSQGDAAVIAAAAAATARGKANVTLNTSGVKANDTGVGPNTTNTAGGANTTANVVANGTANVIVNPSTNATPTGTSNASVTNTTERAVPVVAPTQPSNGYAECRSAITNRTETQNMEPPYDRKHRYEVFLPKKYDFNVSWCVDIIDWRDLDEMLNNRTDEVVEKSLSWCGHGCIIAFAVVGSLIAACLVVLAVVLTSKRRRLAAVVAPPRPKFVSTVEDDEEDRVSNIGVPLTDGKGTTAP

SEQ ID NO: 7 - protein, 750 aa, Trypanosoma brucei
  misc_feature (39)..(39): Xaa can be any naturally occurring amino acid
  misc_feature (124)..(124): Xaa can be any naturally occurring amino acid
  misc_feature (220)..(220): Xaa can be any naturally occurring amino acid
  misc_feature (356)..(356): Xaa can be any naturally occurring amino acid
MPLWKQTNCEVETMNVREVVGTVHLGYVSQMLLLVATVXTIVVRSGAAPIELKRHVTTVAGKYGHIGDKDGFPGMSELSSPHAMCRGRNSDEILLGTVDRFRAFSRKNRETTTITAWETDEDQXSGSRSVKVDKPRACVQWTVGGSTFVYFVESMGEVKYFKDSGVFSHDVVRNGSLTGVALYGNHLYLTEQNTNTVWTCEVGSDGDPIACHSHVALSAXCSIYGPIGIAATQQGIFVVARGPAKQGTICWFDLQGHKIAEVDGEYVDITSTRSGDLLAATQNELHRVSTDGNKLTTKRFAGGSTNSCLPNTEGDDTLLCEITRLLVVTEYEMYVTSEKKSVLRSVTLPPVYVQGXFPGRPLPVGYPDKDIMEWIVGNLTEDINTALGTTESIVASSSVHVDSTTWLTNFTAGVQQPDFDDEKTEQALHKSNYEHTKEAADEYYNLTDEQVYMDSTMVPYCNRLSLDALRRKLAKEAGEVLNFTLIYADMPLKAESSDAGNITTVKLLMPASFNNTVTHDLLSDANLTETAHSFIKYLRSSDTHVDVTFSNPPFNFSSLTPDEEQEVRWYIHDEVMNQIKKCEERSTGRSMARREEVGDYSRTTIATALDSNVTGVCQSTITNRTVSLFYQPPYVEMSLYEVFIPGNYTFDVSECVGEIDWQDLNDHLNNDTVRPTTEKAPKCGRVCLIIIAVVCALIVAVLIVLAVVFTSKRRRLAAVVAPARPKFVSTLDEDEQDYASAYGNKERVEQ

SEQ ID NO: 8 - protein, 750 aa, Trypanosoma brucei; identical to SEQ ID NO: 7
  misc_feature (39)..(39): Xaa can be any naturally occurring amino acid
  misc_feature (124)..(124): Xaa can be any naturally occurring amino acid
  misc_feature (220)..(220): Xaa can be any naturally occurring amino acid
  misc_feature (356)..(356): Xaa can be any naturally occurring amino acid

SEQ ID NO: 9 - protein, 818 aa, Trypanosoma brucei
MCFIFGVEMSNLAKRPMSLRKLPQLFLLIMIGIAFVAVECIGAPVKLPRRVDTVAGQFGVEGETNGYPNTTRLTEPYALCRGRTNDEILVGSSNSFRNYSRKTKETGTYLRYNVGDSVISGSSTINKPRSCVRRGSGNHTIIYFVDDQKDIKYIVGDDVSSFSVPTRGSLNAVAVHEGSLYVTDQNNKSVWKCGLGGAGKPQSCEEKKFTSVTLDAKPEGIAVTSKGIFVAARDSSNKGALLWLDMNGSNRKGNVSGGFVDVFSTESGMLYAATEKELYTVTATDSAFSVTLFAGKNTSQCYFPTNGEDIVLCDNSRLLVIEEYEMYVTSKEKHTMRALTLPPVNLTAIFRGRPAPVGYPNTTIMEQFVASLTEDVNKALGTNDSYVDPDSVRVDPDTWETNFTVFVQQTRFDNTTEEKLRSLTYTQTDKTVDEYYGLTDEYVYIDTVLVPFCDDASLVTIQRALAREAGRALNFSLIYADKPITFGSDVAENVTAVKLLMPHSFKNATTPKQLSAANLTDFAHNLVKDLRASDTRVDITFPDPPFNFSAVVPEREQEVRWFVHGKVMKQLEICERLGSQGDAAVIAAAAAATARGKANVTLNTSGVKANDTGVGPNTTNTAGGANTTANVVANGTANVIVNPSTNATPTGTSNASVTNTTERAVPVVAPTQPSNGYAECRSAITNRTETQNMEPPYDRKHRYEVFLPKKYDFNVSWCVDIIDWRDLDEMLNNRTDEVVEKSLSWCGHGCIIAFAVVGSLIAACLVVLAVVLTSKRRRLAAVVAPPRPKFVSTVEDDEEDRVSNIGVPLTDGKGTTAP

SEQ ID NO: 10 - protein, 818 aa, Trypanosoma brucei
MCFIFGVEMSNLAKRPMSLRKLPQLFLLIMIGIAFVAVECIGAPVKLPRRVDTVAGQFGVEGETNGYPNTTRLTEPYALCRGRTNDEILVGSSNSFRNYSRKTKETGTFLRGGPTGGLVSADAKISKPRSCVRRGSGNHTIIYFVDDQNGLKYINDNEIQHVTVGDGLSLTSVAIYEKDLYVTDQNNKSVWRCNVGGAGKPQNCEEKKFTGLTFTAKPEGIAVTSKGIFVAARDSSNKGALLWLDMNGSNRKGNVSGGFVDVFSTESGMLYAATEKELYTVTATDSAFSVTLFAGKNTSSCYSHANGEDIVLCDNSRLLVIEEYEMYVTSMEKHTMRALTLPPVNLTAIFRGRPAPVGYPNTTIMEQFVASLTEDVNKALGTNDSYVDPDSVRVDPDTWETNFTVFVQQTRFDNTTEEKLRSLTYTQTDKTVDEYYGLTDEYVYIDTVLVPFCDDASLVTIQRALAREAGRALNFSLIYADKPITFGSDVAENVTAVKLLMPHSFKNATTPKQLSAANLTDFAHNLVKDLRASDTRVDITFPDPPFNFSAVVPEREQEVRWFVHGKVMKQLEICERLGSQGDAAVIAAAAAVTARGKANVTLNTSGVKANDTGVGPNTTNTAGGANTTANVVANGTANVIVNPSTNATPTGTSNASATNTTERAVPVVAPTQPSNGYAECRSAITNRTETQNMEPPYDRKHRYEVFLPKKYDFNVSWCVDIIDWRDLDEMLNNRTDEVVEKSLSWCGHGCIIAFAVVGSLIAACLVVLAVVLTSKRRRLAAVVAPPRPKFVSTVEDDEEDRVSNIGVPLTDGKGTTAP

SEQ ID NO: 11 - protein, 810 aa, Trypanosoma brucei
MCFIFGVEMSNLAKRPMSLRKLPQLFLLIMIGIAFVAVECIGAPVKLPRRVDTVAGQFGVEGETNGYPNTTRLTEPYALCRGRTNDEILVGSSNSFRNYSRKTKETGTFLRGGPTGGLVSADAKISKPRSCVRRGSGNHTIIYFVDDQNGLKYINDNEIQHVTVGDGLSLTSVAIYEKDLYVTDQNNKSVWRCNVGGAGKPQNCEEKKFTGLTFTAKPEGIAVTSKGIFVAARDSSNKGALLWLDMNGSNRKGNVSGGFVDVFSTESGMLYAATEKELYTVTATDSAFSVTLFAGKNTSSCYSHANGEDIVLCDNSRLLVIEEYEMYVTSKEKHTMRALTLPPVNLTAIFRGRPAPVGYPNTTIMEQFVASLTEDVNKALGTNDSYVDPDSVRVDPDTWETNYTVFVQQTRFDNTTEEKLRSLTYTQTDKTVDEYYGLTDEYVYIDTVLVPFCDDASLVTIQRALAREAGRALNFSLIYADKPITFGSDVAENVTAVKLLMPHSFKNATTPKQLSAANLTDFAHNLVKDLRASDTRVDITFPDPPFNFSAVVPEREQEVRWFVHGKVMKQLEICERLGSQGDAAVIAAAADATARGKANVTLNTSGVKANDTGVGPNTTNTAGGANTTANVATNGTANVIVNPSTNASVTNTTERAVPVVAPTQPSNGYAECRSAITNRTETQNMEPPYDRKHRYEVFLPKKYDFNVSWCVDIIDWRDLDEMLNNRTDEVVEKSLSWCGHGCIIAFAVVGSLIAACLVVLAVVLTSKRRRLAAVVAPPRPKFVSTVEDDEEDRVSNIGVPLTDGKGTTAP

SEQ ID NO: 12 - protein, 750 aa, Trypanosoma brucei
MPLWKQTNCEVETMNVREVVGTVHLGYVSQMLLLVATVATIVVRSGAAPIELKRHVTTVAGKYGHIGDKDGFPGMSELSSPHAMCRGRNSDEILLGTVDRFRAFSRKNRETTTITAWETDEDQKSGSRSVKVDKPRACVQWTVGGSTFVYFVESMGEVKYFKDSGVFSHDVVRNGSLTGVALYGNHLYLTEQNTNTVWTCEVGSDGDPIACHSHVALSANCSIYGPIGIAATQQGIFVVARGPAKQGTICWFDLQGHKIAEVDGEYVDITSTRSGDLLAATQNELHRVSTDGNKLTTKRFAGGSTNSCLPNTEGDDTLLCEITRLLVVTEYEMYVTSEKKSVLRSVTLPPVYVQGLFPGRPLPVGYPDKDIMEWIVGNLTEDINTALGTTESIVASSSVHVDSTTWLTNFTAGVQQPDFDDEKTEQALHKSNYEHTKEAADEYYNLTDEQVYMDSTMVPYCNRLSLDALRRKLAKEAGEVLNFTLIYADMPLKAESSDAENITTVKLLMPASFNNTVTHDLLSDANLTETAHSFIKYLRSSDTHVDVTFSNPPFNFSSLTPDEEQEVRWYIHDEVMNQIKKCEERSTGRSMARREEVGDHSRTTIATALDSNVTGVCQSTITNRTVSLFYQPPYVEMSLYEVFIPGNYTFDVSECVGEIDWQDLNDHLNNDTVRPTTEKAPKCGRVCLIIIAVVCALIVAVLIVLAVVFTSKRRRLAAVVAPARPKFVSTLDEDEQDYASAYGNKERVEQ

SEQ ID NO: 13 - protein, 750 aa, Trypanosoma brucei
MPLWKQTNCEVETMNVREVVGTVHLGYVSQMLLLVATVETIVVRSGAAPIELKRHVTTVAGKYGHIGDKDGFPGMSELSSPHAMCRGRNSDEILLGTVDRFRAFSRKNRETTTITAWETDEDQNSGSRSVKVDKPRACVQWTVGDSTFVYFVESMGEVKYFKDSGVFSHDVVRNGSLTGVALYGNHLYLTEQNTNTVWTCEVGSDGDPIACHSHVALSAKCSIYGPIGIAATQQGIFVVARGPAKQGTICWFDLQGHKIAEVDGEYVDITSTRSGDLLAATQNELHRVSTDGNKLTTKRFAGGSTNSCLPNTEGDDTLLCEITRLLVVTEYEMYVTSEKKSVLRSVTLPPVYVQGVFPGRPLPVGYPDKDIMEWIVGNLTEDINTALGTTESIVASSSVHVDSTTWLTNFTAGVQQPDFDDEKTEQALHKSNYEHTKEAADEYYNLTDEQVYMDSTMVPYCNRLSLDALRRKLAKEAGEVLNFTLIYADMPLKAESSDAGNITTVKLLMPASFNNTVTHDLLSDANLTETAHSFIKYLRSSDTHVDVTFSNPPFNFSSLTPDEEQEVRWYIHDEVMNQIKKCEERSTGRSMARREEVGDYSRTTIATALDSNVTGVCQSTITNRTVSLFYQPPYVEMSLYEVFIPGNYTFDVSECVGEIDWQDLNDHLNNDTVRPTTEKAPKCGRVCLIIIAVVCALIVAVLIVLAVVFTSKRRRLAAVVAPARPKFVSTLDEDEQDYASAYGNKERVEQ

SEQ ID NO: 14 - protein, 818 aa, Trypanosoma brucei
MCFIFGVEMSNLAKRPMSLRKLPQLLLLIMIGIAFVAVECIGAPVKLPRRVDTVAGQFGVEGETNGYPNTTRLTEPYALCRGRTNDEILVGSSNSFRNYSRKTKETGTYLRYNVGDSVISGSSTINKPRSCVRRGSGNHTIIYFVDDQKDIKYIVGDDVSSFSVPTSGSLNAVAVHEGTLYVTDQNNKSVWKCGLGGAGKPQSCEEKKFTSVTLDAKPEGIAVTSKGIFVTARDSSNKGALLWLDMSGGNRKGNVSGGFVDVFSTESGVLYAATEKELYTVTATDTSLSVTSFAGKNTSQCYFPTNGEDIVLCDNSRLLVIEEYEMYVTSKAKHTMRALTLPPVNLTAIFRGRPAPVGYPNTTIMEQFVASLTEDVNKALGTNDSYVDPDSVRVDPDTWETNFTVFVQQTRFDNTTEEKLRSLTYTQTDKTVDEYYGLTDEYVYIDTVLVPFCDDASLVTIQRALAREAGRALNFSLVYADKPITFGSDVAENVTAVKLLMPHSFKNATTPKQLSAANLTDFAHNLVKDLRASDTRVDITFPDPPFNFSAVVPEREQEVRWFVHGKVMKQLEICERLGSQGDAAVIAAAADATARGKANVTLNTSGVKANDTGVGPNTTNTAGGANTTANVAANGTANVIVNPSTNATPTGTTNASVTNTTERAVPVVAPTQPSNGYAECRSAITNRTETQNMEPPYDRKHRYEVFLPKKYDFNVSWCVDIIDWRDLDEMLNNRTDEVVEKSLSWCGHGCIIAFAVVGSLIAACLVVLAVVLTSKRRRLAAVVAPPRPKFVSTVEDDDEDRVSNIGVPLTDGKGTTAP

SEQ ID NO: 15 - protein, 818 aa, Trypanosoma brucei
MCFIFGVEMSNLAKRPMSLRKLPQLLLLIMIGIAFVAVECIGAPVKLPRRVDTVAGQFGFDGTTDGSSNVSMLSSPYALCRGRTNDEILVGSSNSFRNYSRKTKETGTFLRGGPTGGLVSADAKISKPRSCVRRGSGNHTIIYFVDDQNGLKYINDNEIQHVTVGNGLSLTSVAIYEKDLYVTDQNNKSVWRCNVGGAGKPQNCEEKKFTGLTFTAKPEGIAVTSKGIFVAARDSSNKGALLWLDMNGGGSKGNVSGGFVDVFSTESGMLYAATEKELYTVTATGSAFSVTSFAGKNTSSCYSHANGEDIVLCDNSRLLVIEEYEMYVTSKEKHTMRALTLPPVNLTAIFRGRPAPVGYPNTTIMEQFVASLTEDVNKALGTNDSYVDPDSVRVDPDTWETNFTVFVQQTRFDNTTEEKLRSLTYTQTDKTVDEYYGLTDEYVYIDTVLVPFCDDASLVTIQRALAREAGRALNFSLIYADKPITFGSDVAENVTAVKLLMPHSFKNATTPKQLSAANLTDFAHNLVKDLRASDTRVDITFPDPPFNFSAVVPEREQEVRWFVHGKVMKQLEICERLGSQGDAAVIAAAAAATARGKANVTLNTSGVKANDTGVGPNTTNTAGGANTTANVVANGTANVIVNPSTNATPTGTTNASVTNTTERAVPVVAPTQPSNGYAECRSAITNRTETQNMEPPYDRKHRYEVFLPKKYDFNVSWCVDIIDWRDLDEMLNNRTDEVVEKSLSWCGHGCIIAFAVVGSLIAACLVVLAVVLTSKRRRLAAVVAPPRPKFVSTVEDDEEDRVSNIGVPLTDGKGTTAP

SEQ ID NO: 16 - protein, 750 aa, Trypanosoma brucei
MPLWKQTNCEVETMNVREVVGTVHLGYVSQMLLLVATVATIVVRSGAAPIELKRHVTTVAGKYGHIGDKDGFPGMSELSSPHAMCRGRNSDEILLGTVDRFRAFSRKNRETTTITAWETDEDQKSGSRSVKVDKPRACVQWTVGGSTFVYFVESMGEVKYFKDSGVFSHDVVRNGSLTGVALYGNHLYLTEQNTNTVWTCEVGSDGDPIACHSHVALSANCSIYGPIGIAATQQGIFVVARGPAKQGTICWFDLQGHKIAEVDGEYVDITSTRSGDLLAATQNELHRVSTDGNKLTTKRFAGGSTNSCLPNTEGDDTLLCEITRLLVVTEYEMYVTSEKKSVLRSVTLPPVYVQGLFPGRPLPVGYPDKDIMEWIVGNLTEDINTALGTTESIVASSSVHVDSTTWLTNFTAGVQQPDFDDEKTEQALHESNYEHTKEAADEYYNLTDEQVYMDSTMVPYCNRLSLDALRRKLAKEAGEVLNFTLIYADMPLKAESSDAENITTVKLLMPASFNNTVTHDLLSDANLTETAHSFIKYLRSSDTHVDVTFSNPPFNFSSLTPDEEQEVRWYIHDEVMNQIKKCEERSTGRSMARREEVGDHSRTTIATALDSNVTGVCQSTITNRTVSLFYQPPYVEMSLYEVFIPGNYTFDVSECVGEIDWQDLNDHLNNDTVRPTTEKAPKCGRVCLIIIAVVCALIVAVLIVLAVVFTSKRRRLAAVVAPARPKFVSTLDEDEQDYASAYGNKERVEQ

SEQ ID NO: 17 - protein, 750 aa, Trypanosoma brucei; identical to SEQ ID NO: 16

SEQ ID NO: 18 - protein, 818 aa, Trypanosoma brucei
MCFIFGVEMSNLAKRPMSLRKLPQLLLLIMIGIAFVAVECIGAPVKLPRRVDTVAGQFGVEGETNGYPNTTRLTEPYALCRGRTNDEILVGSSNSFRNYSRKTKETGTYLRYNVGDSVISGSSTINKPRSCVRRGSGNHTIIYFVDDQKDIKYIVGDDVSSFSVPTSGSLNAVAVHEGSLYVTDQNNKSVWKCGLGGAGKPQSCEEKKFTSVTLDAKPEGIAVTSKGIFVTARDSSNKGALLWLDMSGSNRKGNVSGGFVDVFSTESGVLYAATEKELYTVTATDTSLSVTLFAGKNTSQCYFSTNGEDIVLCDNSRLLVIEEYEMYVTSKEKHTMRALTLPPVNLTAIFRGRPAPVGYPNTTIMEQFVASLTEDVNKALGTNDSYVDPDSVRVDPNTWETNYTVFVQQTRFDNTTEEKLRSLTYTQTDKTVDEYYGLTDEYVYIDTVLVPFCDDASLVTIQRALAREAGRALNFSLIYADKPITFGSDVAENVTAVKLLMPHSFKNATTPKQLSAANLTDFAHNLVKDLRASDTRVDITFPDPPFNFSAVVPEREQEVRWFVHGKVMKQLEICERLGSQGDAAVIAAAADATARGKANVTLNTSGVKANDTGVGPNTTNTAGGADTTANVAANGTANVIVNPSTNATPTGTTNASVTNTTERAVPVVAPTQPSNGYAECRSAITNRTETQNMEPPYDRKHRYEVFLPKKYDFNVSWCVDIIDWRDLDEMLNNRTDEVVEKSLSWCGHGCIIAFAVVGSLIAACLVVLAVVLTSKRRRLAAVVAPPRPKFVSTVEDDEEDRVSNIGVPLTDGKGTTAP

SEQ ID NO: 19 - protein, 818 aa, Trypanosoma brucei
MCFIFGVEMSNLAKRPMSLRKLPQLLLLIMIGIAFVAVECIGAPVKLPRRVDTVAGQFGFDGTTDGSSNVSMLSSPYALCRGRTNDEILVGSSNSFRNYSRKTKETGTFLRGGPTGGLVSADAKISKPRSCVRRGSGNHTIIYFVDDQNGLKYINDNEIQHVTVGNGLSLTSVAIYEKDLYVTDQNNKSVWRCNVGGAGKPQNCEEKKFTGLTFTAKPEGIAVTSKGIFVAARDSSNKGALLWLDMNGGGSKGNVSGGFVDVFSTESGMLYAATEKELYTVTATDSAFSVTSFAGKNTSSCYSHANGEDIVLCDNSRLLVIEEYEMYVTSKEKHTMRALTLPPVNLTAIFRGRPAPVGYPNTTIMEQFVASLTEDVNKALGTNDSYVDPDSVRVDPDTWETNFTVFVQQTRFDNTTEEKLRSLTYTQTDKTVDEYYGLTDEYVYIDTVLVPFCDDASLVTIQRALAREAGRALNFSLIYADKPITFGSDVAENVTAVKLLMPHSFKNATTPKQLSAANLTDFAHNLVKDLRASDTRVDITFPDPPFNFSAVVPEREQEVRWFVHGKVMKQLEICERLGSQGDAAVIAAAAAATARGKANVTLNTSGVKANDTGVGPNTTNTAGGANTTANVAANGTANVIVNPSTNATPTGTTNASVTNTTERAVPVVAPTQPSNGYAECRSAITNRTETQNMEPPYDRKHRYEVFLPKKYDFNVSWCVDIIDWRDLDEMLNNRTDEVVEKSLSWCGHGCIIAFAVVGSLIAACLVVLAVVLTSKRRRLAAVVAPPRPKFVSTVEDDEEDRVSNIGVPLTDGKGTTAP

SEQ ID NO: 20 - protein, 820 aa, Trypanosoma brucei
LAMCFIFGVEMSNLAKRPMSLRKLPQLLLLIMIGIAFVAVECIGAPVKLPRRVDTVAGQFGFDGTTDGSSNVSMLSSPYALCRGRTNDEILVGSSNSFRNYSRKTKETGTFLRGGPTGGLVSADAKISKPRSCVRRGSGNHTIIYFVDDQNGLKYINDNEIQHVTVGNGLSLTSVAIYEKDLYVTDQNNKSVWRCNVGGAGKPQNCEEKKFTGLTFTAKPEGIAVTSKGIFVAARDSSNKGALLWLDMNGGGSKGNVSGGFVDVFSTESGMLYAATEKELYTVTATGSAFSVTSFAGKNTSSCYSHANGEDIVLCDNSRLLVIEEYEMYVTSKEKHTMRALTLPPVNLTAIFRGRPAPVGYPNTTIMEQFVASLTEDVNKALGTNDSYVDPDSVRVDPDTWETNFTVFVQQTRFDNTTEEKLRSLTYTQTDKTVDEYYGLTDEYVYIDTVLVPFCDDASLVTIQRALAREAGRALNFSLIYADKPITFGSDVAENVTAVKLLMPHSFKNATTPKQLSAANLTDFAHNLVKDLRASDTRVDITFPDPPFNFSAVVPEREQEVRWFVHGKVMKQLEICERLGSQGDAAVIAAAADATARGKANVTLNTSGVKANDTGVGPNTTNTAGGANTTANVAANGTANVIVNPSTNATPTGTTNASVTNTTERAVPVVAPTQPSNGYAECRSAITNRTETQNMEPPYDRKHRYEVFLPKKYDFNVSWCVDIIDWRDLDEMLNNRTDEVVEKSLSWCGHGCIIAFAVVGSLIAACLVVLAVVLTSKRRRLAAVVAPPRPKFVSTVEDDEEDRVSNIGVPLTDGKGTTAP

SEQ ID NO: 21 - protein, 573 aa, Trypanosoma brucei gambiense
MSGSNRKGNVSGGFVDVFSTESGMLYAATEKELYTVTATDTSLSVTLFAGKNTSSCYSHANGEDIVLCDNSRLLVIEEYEMYVTSKEKHTMRALTLPPVNLTAIFRGRPAPVGYPNTTIMEQFVASLTEDVNKALGTNDSYVDPDSVRVDPDTWETNYTVFVQQTRFDNTTEEKLRSLTYTQTDKTVDEYYGLTDEYVYIDTVLVPFCDDASLVTIQRALAREAGRALNFSLIYADKPITFGSDVAENVTAVKLLMPHSFKNATTPKQLSAANLTDFAHNLVKDLRASDTRVDITFPDPPFNFSAVVPEREQEVRWFVHGKVMKQLEICERLGSQGDAAVIAAAADATARGKANVTLNTSGVKANDTGVGPNTTNTAGGANTTANVATNGTANVIVNPSTNATPTGTSNASATNTTERAVPVVAPTQPSNGYAECRSAITNRTETQNMEPPYDRKHRYEVFLPKKYDFNVSWCVDIIDWRDLDEMLNNRTDEVVEKSLSWCGHGCIIAFAVVGSLIAACLVVLAVVLTSKRRRLAAVVAPPRPKFVSTVEDDEEDRVSNIGVPLTDGKGTTAP

SEQ ID NO: 22 - protein, 818 aa, Trypanosoma brucei gambiense
MCFIFGVEMSNLAKRPMSLRKLPQLFLLIMIGIAFVAVECIGAPVKLPRRVDTVAGQFGFDGTTDGSSNVSMLSSPYALCRGRTNDEILVGSSNSFRNYSRKTKETGTFLRGGPTGGLVSADAKISKPRSCVRRGSGNHTIIYFVDDQNGLKYINDNEIQHVTVGNGLSLNAVAIHEKDLYVTDQNNKSVWRCNVGGAGKPQNCEEKKFTGVTFTAKPEGIAVTSKGIFVTARDSSNKGALLWLDMNGGGRKGNVSGGFVDVFSTESGVLYAATEKELYTVTATDTSLSVTLFAGKNTSQCYFPTNGEDIVLCDNSRLLVIEEYEMYVTSKAKHTMRALTLPPVNLTAIFRGRPAPVGYPNTTIMEQFVASLTEDVNKALGTNDSYVDPDSVRVDPDTWETNYTVFVQQTRFDNTTEEKLRSLTYTQTDKTVDEYYGLTDEYVYIDTVLVPFCDDASLVTIQRALAREAGRALNFSLIYADKPITFGSDVAENVTAVKLLMPHSFKNATTPKQLSAANLTDFAHNLVKDLRASDTRVDITFPDPPFNFSAVVPEREQEVRWFVHGKVMKQLEICERLGSQGDAAVIAAAADATARGKANVTLNTSGVKANDTGVGPNTTNTAGGANTTANVATNGTANVIVNPSTNATPTGTSNASATNTTERAVPVVAPTQPSNGYAECRSAITNRTETQNMEPPYDRKHRYEVFLPKKYDFNVSWCVDIIDWRDLDEMLNNRTDEVVEKSLSWCGHGCIIAFAVVGSLIAACLVVLAVVLTSKRRRLAAVVAPPRPKFVSTVEDDEEDRVSNIGVPLTDGKGTTAP

SEQ ID NO: 23 - protein, 750 aa, Trypanosoma brucei gambiense; identical to SEQ ID NO: 13

SEQ ID NO: 24 - protein, 600 aa, Trypanosoma brucei gambiense
FVESIGEVKYFRDSGVFSHDVVRNGSLTGVALYGNHLYLTEQNTNTVWTCEVGSDGDPIACHSHVALSAKCSIYGPIGIAATQQGIFVVARGPAKQGTICWFDLQGHKIAEVDGEYVDITSTRSGDLLAATQNELHRVSTDGNKLTTKRFAGGSTNSCLPNTEGDDTLLCEITRLLVVTEYEMYVTSEKKSVLRSVTLPPVYVQGVFPGRPLPVGYPDKDIMEWIVGNLTEDINTALGTTESIVASSSVHVDSTTWLTNFTAGVQQPDFDDEKTEQALHKSNYEHTKEAADEYYNLTDEQVYMDSTMVPYCNRLSLDALRRKLAKEAGEVLNFTLIYADMPLKAESSDAGNITTVKLLMPASFNNTVTHDLLSDANLTETAHSFIKYLRSSDTHVDVTFSNPPFNFSSLTPDEEQEVRWYIHDEVMNQIKKCEERSTGRSMARREEVGDYSRTTIATALDSNVTGVCQSTITNRTVSLFYQPPYVEMSLYEVFIPGNYTFDVSECVGEIDWQDLNDHLNNDTVRPTTEKAPKCGRVCLIIIAVVCALIVAVLIVLAVVFTSKRRRLAAVVAPARPKFVSTLDEDEQDYASAYGNKERVEQ

SEQ ID NO: 25 - protein, 151 aa, Trypanosoma cruzi
MELPPPPLPIGYPDDNEVMKKIIQLMNEELNEHLKTNGTYVSQENMYVDANTWATKFAVMVQQHDFENATTPGEVLTTHFAQTKQFVKDYYDRVDEVLYMDTSIMPFCNDTMLNAVMHRLVSVVREVSVFRLFTPTRQRLGRNLILKTSPQ

SEQ ID NO: 26 - protein, 218 aa, Trypanosoma cruzi
MDADMDAALLQILRELYGPENVVTLVFPMPEYDFSKLTDEQLVEIRWFILDMVRARLEECAVLSAGSVDASVSRHSGVCEAVITNRTETVISHPPFNIQSEYEVFVPSRYNFNASLCLDGIDWAVLEEVIKNYTEENKPRHKSACDRSCIIGLAVLAALVLTALIAVVVVLTSKRRRLAAVVAPVHPKFKSTLDEDEEEIETTNPLELKEEQRARDMY

SEQ ID NO: 27 - protein, 712 aa, Trypanosoma cruzi
MFRFQSLLFALFTGLLFSFTSSVVVAMPLRYMVETVSGITGSIGHVNGGPGASLLTRPSAICQGRNEDELLFGTQGYFRNFSRSTKMTGILIGDGTAQILDGTWSQARIDGPRGCVRGIFNQKMIVYFVEGQSSLRYFTSNYVHTVIISINLSFTDVKLYEGKLYITEQTKDEVWVCDIDADGAPVSCALKTGFKCDYGKYHGITVTKLGVFVVGESAAGICHFDMNGNKISVLGGNYIDVFSLPSDELYIMSYTELLHLRVTGSAMAVEKFAGRADATCPPLIDGYDFTLCKNLRLFVIEQSEMYLATTLNTVRSVTLPPAIVWMELPPPPLPIGYPDDNEVMKKIIQLMNEELNEHLKTNGTYVSQENMYVDANTWATKFAVMVQQHDFENATTPGEVLTTHFAQTKQFVKDYYDRVDEVLYMDTSIMPFCNDTMLNAVMHRLVSVVREVLGFPLIYANPPEARKEFNFENITTMKLLMPASFNNDTTRDALMDADMDAALLQILRELYGPENVVTLVFPMPEYDFSKLTDEQLVEIRWFILDMVRARLEECAVLSAGSVDASVSRHSGVCEAVITNRTETVISHPPFNIQSEYEVFVPSRYNFNASLCLDGIDWAVLEEVIKNYTEENKPRHKSACDRSCIIGLAVLAALVLTALIAVVVVLTSKRRRLAAVVAPVHPKFKSTLDEDEEEIETTNPLELKEEQRARDMY

SEQ ID NO: 28 - protein, 708 aa, Trypanosoma cruzi
MSRFQRLLFALFAGFLFSFTASVVVAMPLRYMVETVSGITGSIGHVNGGPGTSLLTRPSAICQGRNEDELLFGTQGYFRNFSRSTKMTGILLGDGTVQILDGTWSQARIDGPRGCVRGIFNQKMIVYFVEGQSSLRYFTSNYVHTVTISINLSFTDVKLYEGKLYITEQTKDEVWGCDIDADGAPVSCALKTGFKCDYGKYHGITVTKLGVFVVGESAAGICHFDMHGNKISVLGGNYIDVFSLPSDELYIMSYTELLHLRVIGSAMVVEKFAGRSDATCPPLIDGYDFTLCKNLRLFVIEQSEMYLATTLNTVRSVTLPPAIVWIELPPPPLPIGYPNDNEVMKKIIQLMNEELNKHLGTNGTYVSQETMHVDANTWATKFAVMVQQQDFENATTPGEVLTTHFARTKQFVKDYYDRVNEVLYMDTSIMPFCNDTMLNAVMHRLVTVVREVLSFPLIYANPPEVRKEFDFENITTMKLLMPASFNNDTTREALMDADMDAALLQILRELYGPEHVVTLVFPMPQYDFSKLTDEQLVEVRWFILDLVRARLEECAVLSVDGVGASVSSHSSVCEAVITNRTETVVSHPPFNIQSEYEVFVPSRYKFNASLCLDGIDWTVLEELIKNYTEENKPRHKSACDRSCIIGLAVLAALVLTALIAVMVVLTSKRRRLAAVVAPVHPKFKSTLDEDEEEMETTNPLEVKDEQRA

SEQ ID NO: 29 - protein, 366 aa, Trypanosoma cruzi
MFWFQSLLFALFAGLLFSFTSSVVVAMPLRYMVETVSGITGSIGHANGGPGTSLLTRPSAICQGRNEDELLFGTQGYFRNFSRSTKMTGILIGDGTAQILDGTWSQARIDGPRGCVRGIFSQKMIVYFVEGQSSLRYFTSDYVHTVMISINLFFTDVKLYEGKLYMTEQTKDEVWGCDIDADGAPVSCALKTGFKCDYGKYHGITVTKQGVFVVGESAAGICHFDMHGNKLSVLGGNYIDVFSLPSDELYIMSYTELFHLRVIGSAMVVEKFAGRADATCPPLIDGYDFTLCKNLRLFVIDQSEMYLATTLNTVRSVTLPPAIVWMELPPPPLPIGYPDEYEVMKRIIQLMNEELNEHLRTNGTYV

SEQ ID NO: 30 - protein, 712 aa, Trypanosoma cruzi
MFRFQSLLFALFTGLLFSFTSSVVVAMPLRYMVETVSGITGSIGHVNGGPGASLLTRPSAICKGRNEDELLFGTQGYFRNFSRSTKMTGILIGDGTAQILDGTWSQARIDGPRGCVRGIFNQKMIVYFVEGQSSLRYFTSNYVHTVIISINLSFTDVKLYEGKLYITEQTKDEVWVCDIDADGAPVSCALKTGFKCDYGKYHGITVTKLGVFVVGESAAGICHFDMNGNKISVLGGNYIDVFSLPSDELYIMSYTELLHLRVTGSAMAVEKFAGRADATCPPLIDGYDFTLCKNLRLFVIEQSEMYLATTLNTVRSVTLPPAIVWMELPPPPLPIGYPDDNEVMKKIIQLMNEELNEHLKTNGTYVSQENMYVDANTWATKFAVMVQQHDFENATTPGEVLTTHFAQTKQFVKDYYDRVDEVLYMDTSIMPFCNDTMLNAVMHRLVSVVREVLGFPLIYANPPEARKEFNFENITTMKLLMPASFNNDTTRDALMDADMDAALLQILWELYGPENVVTLVFPMPEYDFSKLTDEQLVEIRWFILDMVRARLEECAVLSAGSVDASVSRHSGVCEAVITNRTETVISHPPFNIQSEYEVFVPSRYNFNASLCLDGIDWAVLEEVIKNYTEENKPRHKSACDRSCIIGLAVLAALVLTALIAVVVVLTSKRRRLAAVVAPVHPKFKSTLDEDEEEIETTNPLELKEEQRARDMY

SEQ ID NO: 31 - protein, 712 aa, Trypanosoma cruzi; identical to SEQ ID NO: 30

SEQ ID NO: 32 - protein, 712 aa, Trypanosoma cruzi; identical to SEQ ID NO: 30

SEQ ID NO: 33 - protein, 217 aa, Trypanosoma cruzi
MDADMDAALLQILRELYGPENVVTLVFPMPEYDFSKLTDEQLVEIRWFILDMVRARLEECAVLSAGSVDASVSRHSGVCEAVITNRTETVIPHPPFNIQSEYEVFVPSRYNFNASLCLDGIDWAVLEEVIKNYTEENKPRHKSACDRSCIIGLAVLAALVLTALIAVVVVLTSKRRRLAAVVAPVHPKFKSTLDEDEEEIETTNPLELKEEQRARDM

SEQ ID NO: 34 - protein, 353 aa, Trypanosoma cruzi
MIVYFVEGQSSLRYFTSNYVHTVIISINLSFTDVKLYEGKLYITEQTKDEVWVCDIDADGAPVSCALKTGFKCDYGKYHGITVTKLGVFVVGESAAGICHFDMNGNKISVLGGNYIDVFSLPSDELYIMSYTELLHLRVTGSAMAVEKFAGRADATCPPLIDGYDFTLCKNLRLFVIEQSEMYLATTLNTVRSVTLPPAIVWMELPPPPLPIGYPDDNEVMKKIIQLMNEELNEHLKTNGTYVSQENMYVDDNTWATKFAVMVQQHDFENATTPGEVLTTHFAQTKQFVKDYYDRVDEVLYMDTSIMPFCNDTMLNAVMHRLVSVVREVLVFRLFTPTRQRLGRNLILKTSPQ

SEQ ID NO: 35 - protein, 711 aa, Trypanosoma cruzi
MFRFQSLLFALFTGLLFSFTSSVVVAMPLRYMVETVSGITGSIGHVNGGPGASLLTRPSAICQGRNEEELLFGTQGYFRNFSRSTKMTGILIGDGTAQILDGTWSQARIDGPRGCVRGIFNQKMIVYFVEGQSSLRYFTSNYVHTVIISINLSFTDVKLYEGKLYITEQTKDEVWVCDIDADGAPVSCALKTGFKCDYGKYHGITVTKLGVFVVGESAAGICHFDMNGNKISVLGGNYIDVFSLPSDELYIMSYTELLHLRVTGSAMAVEKFAGRADATCPPLIDGYDFTLCKNLRLFVIEQSEMYLATTLNTVRSVTLPPAIVWMELPPPPLPIGYPDDNEVMKKIIQLMNEELNEHLKTNGTYVSQENMYVDANTWATKFAVMVQQHDFENATTPGEVLTTHFAQTKQFVKDYYDRVDEVLYMDTSIMPFCNDTMLNAVMHRLVSVVREVLGFPLIYANPPEARKEFNFENITTMKLLMPASFNNDTTRDALMDADMDAALLQILRELYGPENVVTLVFPMPEYDFSKLTDEQLVEIRWFILDMVRARLEECAVLSAGSVDASVSRHSGVCEAVITNRTETVIPHPPFNIQSEYEVFVPSRYNFNASLCLDGIDWAVLEEVIKNYTEENKPRHKSACDRSCIIGLAVLAALVLTALIAVVVVLTSKRRRLAAVVAPVHPKFKSTLDEDEEEIETTNPLELKEEQRARDM

SEQ ID NO: 36 - protein, 712 aa, Trypanosoma cruzi
MSRFQRLLFALFAGFLFSFTASVVVAMPLRYMVETVSGITGSIGHVNGGPGTSLLTRPSAICQGRNEDELLFGTQGYFRNFSRSTKMTGILLGDGTVQILDGTWSQARIDGPRGCVRGIFNQKMIVYFVEGQSSLRYFTSNYVHTVTISINLSFTDVKLYEGKLYITEQTKDEVWGCDIDADGAPVSCALKTGFKCDYGKYHGITVTKLGVFVVGESAAGICHFDMHGNKISVLGGNYIDVFSLPSDELYIMSYTELLHLRVIGSAMVVEKFAGRSDATCPPLIDGYDFTLCKNLRLFVIEQSEMYLATTLNTVRSVTLPPAIVWIELPPPPLPIGYPNDNEVMKKIIQLMNEELNKHLGTNGTYVSQETMHVDANTWATKFAVMVQQQDFENATTPGEVLTTHFARTKQFVKDYYDRVNEVLYMDTSIMPFCNDTMLNAVMHRLVTVVREVLSFPLIYANPPEVRKEFDFENITTMKLLMPASFNNDTTREALMDADMDAALLQILRELYGPEHVVTLVFPMPQYDFSKLTDEQLVEVRWFILDLVRARLEECAVLSVDGVGASVSSHSSVCEAVITNRTETVVSHPPFNIQSEYEVFVPSRYKFNASLCLDGIDWTVLEELIKNYTEENKPRHKSACDRSCIIGLAVLAALVLTALIAVMVVLTSKRRRLAAVVAPVHPKFKSTLDEDEEEMETTNPLEVKDEQRARDMY

SEQ ID NO: 37 - protein, 712 aa, Trypanosoma cruzi
MFWFQSLLFALFAGLLFSFTSSVVVAMPLRYMVETVSGITGSIGHANGGPGTSLLTRPSAICQGRNEDELLFGTQGYFRNFSRSTKMTGILIGDGTAQILDGTWSQARIDGPRGCVRGIFSQKMIVYFVEGQSSLRYFTSDYVHTVMISINLFFTDVKLYEGKLYMTEQTKDEVWGCDIDADGAPVSCALKTGFKCDYGKYHGITVTKQGVFVVGESAAGICHFDMHGNKLSVLGGNYIDVFSLPSDELYIMSYTELFHLRVIGSAMVVEKFAGRADATCPPLIDGYDFTLCKNLRLFVIDQSEMYLATTLNTVRSVTLPPAIVWMELPPPPLPIGYPDDNEVMKRIIQLMNEELNEHLRTNGTYVSQENMHVDADTWATKFAVMVQQHDFENATTPGEVLTTHFAQTTQFVKDYYDRVDEVLYMDTSIMPFCNDTMLNAVMHRLVSVVREVLGFPLIYANPPEARKELNFENITTMKLLMPASFNNDTTRDALMDADMDAALLQILRELYGPENVVTLVFPMPQYDFSKLTDEQLVEVRWFILDLVRARLEECDVLSASSVDASVSSHSGVCEAVITNRTETVVSHPPFNVQSEYEVFVPLRYKFNASLCLDGIDWAVLEEIIKNYTEENKPRRKSACDRSCIIGLAVLAALVLTALIAVVVVLTSKRRRLAAVVAPVHPKFKSTLDEDEEEIETTNPLEVKDEQRARDMY

SEQ ID NO: 38 - protein, 342 aa, Trypanosoma cruzi
MHVDANTWATKFAVMVQQQDFENATTPGEVLTTHFTRTKQFVKDYYDRVNEVLYMDTSIMPFCNDTMLNAVMHRLVSVVREVLSFPLIYANPPEVRKEFDFENITTMKLLMPASFNNDTTREALMDADMDAALLQILRELYGPEHVVTLVFPMPQYDFSKLTDEQLVEVRWFILDLVRARLEECAVLSVDGVGASVSSHSSVCEAVITNRTETVVSHPPFNIQSEYEVFVPSRYKFNASLCLDGIDWAVLEELIKNYTEENKPRHKSACDRSCIIGLAVLAALVLTALIAVMVVLTSKRRRLAAVVAPVHPKIKSTLDEDEEEMETTNPLEVKDEQRARDMY

SEQ ID NO: 39 - protein, 712 aa, Trypanosoma cruzi
MSRFQSLLFALFAGFLFSFTASVVVAMPLRYMVETVSGITGSIGHVNGGPGTSLLTRPSAICQGRNEDELLFGTQGYFRNFSRSTKMTGILLGDGTAQILDGTWSQARIDGPRGCVRGIFNQKMIVYFVEGQSSLRYFTSNYVHTVTISINLSFTDVKLYEGKLYITEQTKDEVWGCDIDADGAPVSCALKTGFKCDYGKYHGITVTKLGVFVVGESAAGICHFDMHGNKISVLGGNYIDVFSLPSDELYIMSYTELLHLRVIGSAMVVEKFAGRSDATCPPLTDGYDFTLCKNLRLFVIEQSEMYLATTLNTVRSVTLPPAIVWIELPPPPLPIGYPNDNEVMKKIIQLMNEELNKHLGTNGTYVSQETMHVDANTWATKFAVMVQQQDFENATTPGEVLTTHFTRTKQFVKDYYDRVNEVLYMDTSIMPFCNDTMLNAVMHRLVSVVREVLSFPLIYANPPEVRKEFDFENITTMKLLMPASFNNDTTREALMDADMDAALLQILRELYGPEHVVTLVFPMPQYDFSKLTDEQLVEVRWFILDLVRARLEECAVLSVDGVGASVSSHSSVCEAVITNRTETVVSHPPFNIQSEYEVFVPSRYKFNASLCLDGIDWAVLEELIKNYTEENKPRHKSACDRSCIIGLAVLAALVLTALIAVMVVLTSKRRRLAAVVAPVHPKIKSTLDEDEEEMETTNPLEVKDEQRARDMY

SEQ ID NO: 40 - protein, 599 aa, Trypanosoma cruzi
QGIINDKTIIYFVEGQSSLRYITADYVHTVTISTKLSFTDVKLYGGKLYMTEQTKDEIWVCDIDMNGVPVTCVLQNGFKCDYGKYHGITVTKLGVFVVGESASGICHFDMHGNKISVLGGNYVDVYSLPTDTLFVMSFTELLHLRVVGSMMVVEKFAGRVDATCPPLLDGYDFTLCMNLRLFVIDQNEMYLATKLNTVRSITLPPVVVWMELPPPPFPLGFPDDDEKKENVMHKIIQLMNEELNAHLRTQGTYVSLETMQVNDDTWNTKFAVMVQQQDFESATTPAEVLSTDFVQTKKFIMDYYNRVNEVLYMDTSIIPFCDQTMLLQMMHKLVAIVRNVLGFPLIYANPPEALKDFHIENITIMKLLMPASFNNDTTRDALMDTDMDAALLQVLRELYGPDHVVTLIFPMPKYEFSKLTDEQLIQVRWFILDLVRARLAECEILSVDSMDTSSQGTMCEATITNRTETVVPTPPFNLQSEYEVFVPSRYKFNVSVCLDGIDWVALEELIANYTEENKPRHKSACDRSCIIGLAVLTALVLTALIAVLVVLTSKRRRLAAVVAPVHPKFKSTLDEEEEEEMETSNPLEVKEEERTRDTY

SEQ ID NO: 41 - protein, 807 aa, Trypanosoma evansi
MCFIFGVEMSNLAKRPMSLRKLPQLFLLIMIGIAFVAVECIGTPVKLPRRVDTVAGQFGVEGETNGYPNTTRLTEPYALCRGRTNDEILVGSSNSFRNYSRKTKETGTYLRYNVGDSVISGSSTINKPRSCVRRGSGNHTIIYFVDDQKDIKYIVGDDVSSFSVPTRGSLNAVAVHEGSLYVTDQNNKSVWKCGLGGAGKPQSCEEKKFTSVTLDAKPEGIAVTSKGIFVTARDSSNKGALLWLDMSGSNRKGNVSGGFVDVFSTESGMLYAATEKELYTVTATDTSLSVTLFAGKNTSQCYFPTNGEDIVLCDNSRLLVIEEYEMYVTSRAKHTMRALTLPPVNLTAIFRGRPAPVGYPNTTIMEQFVASLTEDVNKALGTNDSYVDPDSVRVDPDTWETNYTVFVQQTRFDNTTEEKLRSLTYTQTDKTVDEYYGLTDEYVYIDTVLVPFCDDASLVTVDGRRAGYADKPITFGSDVAENVTAVKLLMPHSFKNATTPKQLSAANLTDFAHNLVKDLRASDTRVDITFPDPPFNFSAVVPEREQEVRWFVHGKVMKQLEICERLGSQGDAAVIAAAADATARGKANVTLNTSGVKANDTGVGPNTTNTAGGANTTANVATNGTANVIVNPSTNATPTGTTNASVTNTTERAVPVVAPTQPSNGYAECRSAITNRTETQNMEPPYDRKHRYEVFLPKKYDFNVSWCVDIIDWRDLDEMLNNRTDEVVEKSLSWCGHGCIIAFAVVGSLIAACLVVLAVVLTSKRRRLAAVVAPPRPKFVSTVEDDEEDRVSNIGMPLTDGKGTTAP

SEQ ID NO: 42 - protein, 818 aa, Trypanosoma evansi
MCFIFGVEMSNLAKRPMSLRKLPQLLLLIMIGIAFVAVECIGAPVKLPRRVDTVAGQFGFDGTTDGSSNVSMLSSPYALCRGRTNDEILVGSSNSFRNYSRKTKETGTFLRGGPTGGLVSADAKISKPRSCVRRGSGNHTIIYFVDDQNGLKYINDNEIQHVTVGNGLSLTSVAIYEKDLYVTDQNNKSVWRCNVGGAGKPQNCEEKKFTGLTFTAKPEGIAVTSKGIFVAARDSSNKGALLWLDMNGGGSKGNVSGGFVDVFSTESGMLYAATEKELYTVTATDTSFSVTSFAGKNTSSCYSHANGEDIVLCDNSRLLVIEEYEMYVTSKEKHTMRALTLPPVNLTAIFRGRPAPVGYPNTTIMEQFVASLTEDVNKALGTNDSYVDPDSVRVDPDTWETNYTVFVQQTRFDNTTEEKLRSLTYTQTDKTVDEYYGLTDEYVYIDTVLVPFCDDASLVTIQRALAREAGRALNFSLIYADKPITFGSDVAENVTAVKLLMPHSFKNATTPKQLSAANLTDFAHNLVKDLRASDTRVDITFPDPPFNFSAVVPEREQEVRWFVHGKVMKQLEICERLGSQGDAAVIAAAADATARGKANVTLNTSGVKANDTGVGPNTTNTAGGANTTANVVANGTANVIVNPSTNATPTGTSNASVTNTTERAVPVVAPTQPSNGYAECRSAITNRTETQNMEPPYDRKHRYEVFLPKKYDFNVSWCVDIIDWRDLDEMLNNRTDEVVEKSLSWCGHGCIIAFAVVGSLIAACLVVLAVVLTSKRRRLAAVVAPPRPKFVSTVEDDEEDRVSNIGVPLTDGKGTTAP

SEQ ID NO: 43 - protein, 750 aa, Trypanosoma evansi; identical to SEQ ID NO: 13

SEQ ID NO: 44 - protein, 750 aa, Trypanosoma evansi; identical to SEQ ID NO: 13

SEQ ID NO: 45 - protein, 756 aa, Leishmania aethiopica
MGRCIRRVPAAAAAALLLALVAAAAVSTTTARAYDHAGITVAGAIMVGQNLQGKAGASRILNPFAICANFDTADVEDTTLLIGGASYFFTLNRYSTYLGFWYGQGSVNLNSGPIDKVRLTGVFGCVTLRPNSSNSLVTSTVYYVQNDGMLYWVSNSVVYLTPVKHGISFVDVTVHDNNVYLLSTQNHIYRCGIGAGGAVVGSACTQITLTGSTKFDQLITTPSDFRGFVVSSCGIFIAPNSDLYWFNLSGVFIAKSAGVTFVDIKLTSNRDTANRGTPVLMAASTSAVYAVTASSATISYTLVSGKETKSCNPALNNVDSDTSPTFCGIARIYPLSTDMVYMTTGGASVVRAIIVGNTTISDTITRTPFPVYFLDNPSIIPLILDGMNYELVGNSNIPFPYVAINHSTPEVDDGTWDTTFSVDVSNRFFSTVSSAAVVSTPFMGSLHGLQAYYNRTNQILFGDPNVLPMCNLTKMQMIERAVAADARAALQYPYIYTSKAQNFTVNAHPNLTLLKLLMPYPFGEILNESGFFENTTTPAALANVHFNTTMLAAVRNAYTPDFVYDCIFAGNAFPFHILTAAQQQLVRWIIYTAIQEQLAKCAENSPSYTGSDSSSSDSHDDMVPGCVPRVGIGNLTELVMPGMPYSNYNITVFIPEGLHYNFSISRCLDGTDWTNVTDYLQHATTPRTRQCGTGCIVSIAVVSAVVAAILVVAIVIATSKRRRLATVVAPAFTVEPKFASTLDMSSEEGSRNPLNG

SEQ ID NO: 46 - protein, 755 aa, Leishmania amazonensis
MGRSIRRVSAAAAALLMALVAAAAVAPTTARAYDHAGITVAGALMVGQNLQGTAATSRILNPFAICANFDTADVEDTTLLIGGASYFFTLNRYSTYLGFWYGQGSMNLNSGPIDKVRLTGVFGCVTLRPNPSNGLPTSIVYYVQNDGFLYWVSNSIVYLTQVESGISLFDVTVYNNSVYLLSAQNVIYRCGIGAGGAVVGSACTQILLTGSPAFHQLIAVSSDFRGFAVSASGIVVAPTADLFWFNLSGAFISKSAGVTFVDAKFTTNRDTANRGAPVLMAASTSAVYTVATSGPSITYTLVSGEETGRCNPALNNVDSDTSPTFCGIARIYPLSTDMVYMTTGGASVVRAILVGNTTVHDTITRTPFPVYFLDNSSIMPLMLDGMNYELVANSDIPFPYVAINEFTPEVGDSTWDTSFSVDVSNRFFSTASSAAVISTPFMGSLHGLQAYYNRTNQILFGDPNVLPMCNLTKMHMIERAVAADARAALQYPYIYTSRAQNFTVNAQPNLTLLKLLMPYPFGEILNESGFFENTTTPAALANVHFNKTMLAAVRNAYAPDFVYDSIFAGNAFPFHILTAAQQQVVRWVIYMAIQEQLAKCAENTPSYPDSDSSSSDSHDDMVPGCVPRVGISNLTEQMIPGLPYSNFNITFFIPESLHYTFSISRCLDGTDWTNVTDYLQNATITSTRECGTGCIVSIAVASAVVAAILVVVIVIVTSKRRRLATVVAPALTVEPKFASTLDVTSEEGSRNPLNG

SEQ ID NO: 47 - protein, 756 aa, Leishmania braziliensis
MGRCVYRVSSAAATLLTVLIAAVTVAATTARAYDHAGVTVAGALLVGQNEEGKLGTNRILNPFALCANFDTTDVTDTTLLIGGASYFFTFDRHSTYLSFWYGQGSMNLNSGPIDQVRLTGVFGCTTVRTTSSSGSPISAVYYVQNDGFLYWVMNSVVYVTLVKNGISLFDVTVYKGNLYLLSAQNRIYKCLIGPGGAVTGSACTQVMLTGSTAYANLSETSTSEFKGFAVSSAGIFIAPSSSLYWFNLAGGYIASTTTAVVFVDVKFTSNRDTANPGIPTLMAASTSAVYRVSTAGTSITYTLIAGKETATCNLALDNVDSLTDPSFCGIARIYPLSLDVVYMTTAGASVVRAIVVSNTTIRDTITRTPFPLYFLDRASTIPVLLDGMNYEIVGHSNVPFPYVAIDQSTPEVNDTTWDTSFGVDISNRFFSMASSAAVTSTPFMGSLHGLETYYNRTNQIIFGDPNVLPMCNLTKMLMIERAVATAARAALKYPYIYTSNAQNFTVNAQPNLTLVKLLMPYAFGEILNELGFFENTTTAAALAAVQFNTTMLAAVRSAYMMDRVYDCIFSGNAYPFHVLTAAQQQEVRWIIYSAIQNQLARCNQSIPYLVPDSNSSNSYNNMAPGCVPRIGINNLTEMLLPGLPYSNFNITVFIPESLYYNFGISRCLDGTDWTDVMGYLINATTRNNRKCNTGCIVGIAVASALVASFLVVAIVIMTSKRRRLATVVAPAATSEPKFISTLDMTSEEGSRNPLTR

SEQ ID NO: 48 - protein, 756 aa, Leishmania braziliensis
MGRCVYRVSSAAATLLTVLIAAVTVAATTARAYDHAGVTVAGALLVGQNEEGKLGTNRILNPFALCANFDTTDVTDTTLLIGGASYFFTFDRHSTYLSFWYGQGSMNLNSGPIDQVRLTGVFGCTTVRTTSSSGSPISTVYYVQNDGFLYWVMNSVVYVTLVKNGISLFDVTVYKGNLYLLSAQNRIYKCLIGPGGAVTGSACTQVMLAGSTAYANLSETSTSEFKGFAVSSAGIFIAPSSSLYWFNLAGGYIASTTTAVVFVDVKFTSNRDTANPGIPTLMAASTSAVYRVSTAGTSITYTLIAGKETATCNLALDNVDSLTDPSFCGIARIYPLSLDVVYMTTAGASVVRAIVVSNTTIRDTITRTPFPLYFLDRASTIPVLLDGMNYEIVGHSNVPFPYVAIDQSTPEVNDTTWDTSFGVDISNRFFSMASSAAVTSTPFMGSLHGLETYYNRTNQIIFGDPNVLPMCNLTKMLMIERAVATAARAALKYPYIYTSNAQNFTVNAQPNLTLVKLLMPYAFGEILNELGFFENTTTAAALAAVQFNTTMLAAVRSAYMMDRVYDCIFSGNAYPFHVLTAAQQQEVRWIIYSAIQNQLARCNQSIPYLGPDSNSSNSYNNMAPGCVPRIGINNLTEMLLPGLPYSNFNITVFIPESLYYNFGISRCLDGTDWTDVMGYLINATTRNNRKCNTGCIVGIAVASALVASFLVVAIVIMTSKRRRLATVVAPAATSEPKFISTLDMTSEEGSRNPLTR

SEQ ID NO: 49 - protein, 756 aa, Leishmania braziliensis; identical to SEQ ID NO: 48

SEQ ID NO: 50 - protein, 756 aa, Leishmania donovani
MGRCIRRVSSAAAAALLIALAAAAAVATTTARAYDHAGITVAGALLVGQNLQGKAGASRILNPFAICADFDTADVEDTTLLIGGASYFFSFNRYSTYLGFWYGQGSMNLNSGPIDKVRLTGVFGCVTLRPNSSNGLVTSTVYYVQNDGMLYWVSNSVVYLTQVKHGISFVDVTVHDNNVYLLSTQNGIYQCGIGAGGAVVGSACTQITLTGSTEFHQLIPISSDFRGFAVSSSGIFITPTSDLYWFNLSGAFIAKSVGVTFVDTKFTSSRDTANRGTSVLMAASTSAVYTVTTSDATISYALVSGKEVKSCNPALNNVDSDTSPTFCGIARIYPLSTDMVYMTTGGASVVRAIIVSNTTISDTITRTPFPVYFLDNSSIMPLILDGMNYELVSNSNIPFPYVAINHSTPKVDDSTWDTIFSVDVSNRFFSTVSRAAVIGTPFMSSLHGLQAYYNRTNHILFGDPNVLPMCNLTKMQMIERAVAADARAALQYPYIYTSKAQNFTVNAHPNLTLLKLLMPYPFGEILNESGFFENTTTPASLANVHFNTTMLAAVRNAYTPDFVYDCIFAGNAFPFHILTAAQQQLVRWIIYTAIQEQLAKCAENSPSYTGSGSSSSDSHDDMVPGCVPRVGIDNLTELVMPGMPYSNYNITVFIPESLHYNFSISRCLDGTDWTNVTDYLQNATTTRTRKCGTGCIVSIAVVSAVVAAILVVAIVIATSKRRRLATVVAPALTVEPKFASTLDMGSEEGSRNPLNG

SEQ ID NO: 51 - protein, 756 aa, Leishmania donovani
MGRCIRRVSSAAAAALLIALAAAAAVATTTARAYDHAGITVAGALLVGQNLQGKAGASRILNPFAICADFDTADVEDTTLLIGGASYFFSFNRYSTYLGFWYGQGSMNLNSGPIDKVRLTGVFGCVTLRPNSSNGLVTSTVYYVQNDGMLYWVSNSVVYLTQVKHGISFVDVTVHDNNVYLLSTQNGIYQCGIGAGGAVVGSACTQITLTGSTEFHQLIPISSDFRGFAVSSSGIFITPTSDLYWFNLSGAFIAKSVGVTFVDTKFTSSRDTANRGTSVLMAASTSAVYTVTTSDATISYALVSGKEVKSCNPALNNVDSDTSPTFCGITRIYPLSTDMVYMTTGGASVVRAIIVSNTTISDTITRTPFPVYFLDNSSIMPLILDGMNYELVSNSNIPFPYVAINHSTPKVDDSTWDTIFSVDVSNRFFSTVSRAAVIGTPFMSSLHGLQAYYNRTNHILFGDPNVLPMCNLTKMQMIERAVAADARAALQYPYIYTSKAQNFTVNAHPNLTLLKLLMPYPFGEILNESGFFENTTTPASLANVHFNTTMLAAVRNAYTPDFVYDCIFAGNAFPFHILTAAQQQLVRWIIYTAIQEQLAKCAENSPSYTGSGSSSSDSHDDMVPGCVPRVGIDNLTELVMPGMPYSNYNITVFIPESVHYNFSISRCLDGTDWTNVTDYLQNATTTRTRKCGTGCIVSIAVVSAVVAAILVVAIVIATSKRRRLATVVAPALTVEPKFASTLDMGSEEGSRNPLNG

SEQ ID NO: 52 - protein, 756 aa, Leishmania donovani
MGRCIRRVSSAAAAALLIALAAAAAVATTTARAYDHAGITVAGALLVGQNLQGKAGASRILNPFAICADFDTADVEDTTLLIGGASYFFSFNRYSTYLGFWYGQGSMNLNSGPIDKVRLTGVFGCVTLRPNSSNGLVTSTVYYVQNDGMLYWVSNSVVYLTQVKHGISFVDVTVHDNNVYLLSTQNGIYQCGIGAGGAVVGSACTQITLTGSTEFHQLIPISSDFRGFAVSSSGIFITPTSDLYWFNLSGAFIAKSVGVTFVDTKFTSSRDTANRGTSVLMAASTSAVYTVTTSDATISYALVSGKEVKSCNPALNNVDSDTSPTFCGIARIYPLSTDMVYMTTGGASVVRAIIVSNTTVSDTITRTPFPVYFLDNSSIMPLILDGMDYELVSNSNIPFPYVAINHSTPKVDDSTWDTIFSVDVSNRFFSTVSRAAVIGTPFMSSLHGLQAYYNRTNHILFGDPNVLPMCNLTKMQMIERAVAADARAALQYPYIYTSKAQNFTVNAHPNLTLLKLLMPYPFGEILNESGFFENTTTPASLANVHFNTTMLAAVRNAYTPDFVYDCIFAGNAFPFHILTAAQQQLVRWIIYTAIQEQLAKCAENSPSYTGSGSSSSDSHDDMVPGCVPRVGIDNLTELVMPGMPYSNYNITVFIPESLHYNFSISRCLDGTDWTNVTDYLQNATTTRTRKCGTGCIVSIAVVSAVVAAILVVAIVIATSKRRRLATVVAPALTVEPKFASTLDMGSEEGSRNPLNG

SEQ ID NO: 53 - protein, 756 aa, Leishmania infantum
MGRCIRRVSSAAAAALLIALAAAAAVATTTARAYDHAGITVAGALLVGQNLQGKAGASRILNPFAICADFDTADVEDTTLLIGGASYFFSFNRYSTYLGFWYGQGSMNLNSGPIDKVRLTGVFGCVTLRPNSSNGLVTSTVYYVQNDGMLYWVSNSVVYLTQVKHGISFVDVTVHDNNVYLLSTQNGIYQCGIGAGGAVVGSACTQITLTGSTEFHQLIPISSDFRGFAVSSSGIFITPTSDLYWFNLSGAFIAKSVGVTFVDTKFTSSRDTANRGTSVLMAASTSAVYTVTTSDATISYALVSGKEVKSCNPALNNVDSDTSPTFCGIARIYPLSTDMVYMTTGGASVVRAIIVSNTTVSDTITRTPFPVYFLDNSSIMPLILDGMNYELVSNSNIPFPYVAINHSTPKVDDSTWDTIFSVDVSNRFFSTVSRAAVIGTPFMSSLHGLQAYYNRTNHILFGDPNVLPMCNLTKMQMIERAVAADARAALQYPYIYTSKAQNFTVNAHPNLTLLKLLMPYPFGEILNESGFFENTTTPASLANVHFNTTMLAAVRNAYTPDFVYDCIFAGNAFPFHILTAAQQQLVRWIIYTAIQEQLAKCAENSPSYTGSGSSSSDSHDDMVPGCVPRVGIDNLTELVMPGMPYSNYNITVFIPESLHYNFSISRCLDGTDWTNVTDYLQNATTTRTRKCGTGCIVSIAVVSAVVAAILVVAIVIATSKRRRLATVVAPALTVEPKFASTLDMGSEEGSRNPLNG

SEQ ID NO: 54 - protein, 757 aa, Leishmania major
MGRCIRRVSALKAAAALLLALVAAAAVATTTARAYDHAGITVAGAILVGQNLQGKAGASRILNPFAICANFDTTDAEDTTLLIGGASYFFTLNRYSTYLGFWYGQGSMNLNNGPIDKVRLTGVFGCVTLRPNSSNGLVTSTVYYVQNDGMLYWVSNSVVYLTQVKYGISFVDVTVHDNNVYLLSNQNEIYWCGIGAGGTVVGSACTHITLTGSTEFHQLITVSSDFRGFAVSSSGIFIAPTSDLYWFNLSGVFIAKSAGVTFVDTKFTSSRDTVNRETPVLMAASTSAVYSVTTSGATISYTLVSGKETKSCNPALSNVDSDTSPTFCGIARIYPLNTDTVYMTTGGASVVRAIIVSNTTISDTITRTPFPVYFLDNSSIIPLILDGMNYELVGNSNIPFPYVAINHSTPEVDDSTWDTTFSVDVSNRFFSSVSSAAVVSTPFMGSLHGLQAYYNRTNQILFGDPNVLPMCNLTKMLMIERAVAADARAALQYPYIYTSKAQNFTVNAHPNLTLLKLLMPYPFGEILNESGFFENTTTPAALANVHFNTTMLAAVRNAYTPDFVYDCIFAGNAFPFYILTAAQQQLVRWIIYTAIQEQLAKCAENNPSYTGSDSSSSDSHDDMVPGCVPRVGISNLTELVMPGMPYSNYNIAVFIPESLHYNFSISRCLDGTDWTNVTEYLQQATVARTRKCGTGCIVSIAVVSAVVAAILVVAIVIATSKRRRLATVVAPALTVEPKFASTLDVSSEEGSRNPLNG

SEQ ID NO: 55 - protein, 757 aa, Leishmania major; identical to SEQ ID NO: 54

SEQ ID NO: 56 - protein, 757 aa, Leishmania major; identical to SEQ ID NO: 54

SEQ ID NO: 57 - protein, 755 aa, Leishmania mexicana
MGRCIRRVSAAAAALLMALVAAAAVAPTTARAYDHAGITVAGALMVGQNLQGTAGTSRILNPFAICANFDTADVEDTTLLIGGASYFFTLNRYSTYLGFWYGQGSMNLNSGPIDKVRLTGVFGCVTLRPNPSNALPTSIVYYVQNDGFLYWVSNSIVYLTQLESGISLFDVTVYNNSVYLLSAQNVIYRCGIGAGGAVVGSACTQIPLTGSPAFHQLITVSSDFRGFAVSASGIVVAPTADLFWFDLSGAFISKSAGVTFVDAKFTTNRDTANRGAPVLMAASTSAVYTVATSGASITYTLVSGEETGRCNPALNNVDSDTSPTFCGIARIYPLSTDMVYMTTGGASVVRAILVGNTTVRDTITRTPFPVYFLDNSSIMPLMLDGMNYELVANSDIPFPYVAINESTPEVADSTWDTSFSVDVSNRFFSTASSAAVTSTPFMGSLHGLQAYYNRTNQILFGDPNVLPMCNLTKMHMIERAVAADARAALQYPYIYTSRAQNFTVNAQPNLTLLKLLMPYPFGEILNESGFFENTTTPAALANVHFNKTMLAAVRNAYAPDFVYDSIFAGNAFPFHILTAAQQQVVRWVIYMAIQEQLAKCAENTPSYPGSDSSSSDSQDDMVPGCVPRVGISNLTEQMIPGLPYSNFNITVFIPESLHYTFSISRCLDGTDWTNVTDYLQNATITTTRKCGTGCIVSIAVASAVVAAILVVAIVIVTSKRRRLATVVAPALTVEPKFASTLDATSEEGSRNPLNG

SEQ ID NO: 58 - protein, 756 aa, Leishmania panamensis
MGRCVYRVSSAAATLLTVLIAAVTVAATTARAYDHAGVTVAGALLVGQNEEGKLGTNRILNPFALCANFDTTDVTDTALLIGGASYFFTFDRHSTYLSFWYGQGSMNLNSGPIDQVRLTGVFGCTTVRTTSSSGSPISTVYYVQNDGFLYWVMNSVVYVTLVKNGISLFDVTVYKGNLYLLSAQNSIYKCLIGPGGAVTGSACTQVMLAGSTAYANLSETSTSEFKGFAVSSAGIFIAPSSSLYWFNLAGGYIASTTTAVVFVDVKLTSNRDTANPGIPTLMAASTSAVYRVSTAGTSITYTLIAGKETATCNLALDNVDSLTDPSFCGIARIYPLSLDVVYMTTAGASVVRAIVVSNTTIRDTITRTPFPLYFLDRASTIPVLLDGMNYEIVGHSNVPFPYVAIDQSTPEVNDTTWDTSFGVDISNRFFSMASSAAVTSTPFMGSLHGLETYYNRTNQIIFGDPNVLPMCNLTKMLMIERAVATAARAALKYPYIYTSNAQNFTVNAQPNLTLVKLLMPYAFGEILNELGFFENTTTAAALAAVQFNTTMLAAVRSAYMMDRVYDCIFSGNAYPFHVLTAAQQQEVRWIIYSAIQNQLARCNQSIPYLGPDSNSSNSYNNMAPGCVPRIGINNLTEMLLPGLPYSNFNITVFIPESLYYNFGISRCLDGTDWTDVMGYLINATTRNNRKCNTGCIVGIAVASALVASFLVVAIVIMTSKRRRLATVVAPAATSEPKFISTLDMTSEEGSRNPLTR

SEQ ID NO: 59 - protein, 756 aa, Leishmania panamensis; identical to SEQ ID NO: 58

SEQ ID NO: 60 - protein, 757 aa, Leishmania tropica
MGRCIRRVPAAAAAAALLLALVAAAAVSTTTARAYDHAGITVAGAIMVGQNLQGKAGASRILNPFAICANFDTADVEDTTLLIGGASYFFTLNRYSTYLGFWYGQGSMNLNSGPIDKVRLTGVFGCVTLRPNSSNGLVTSTVYYVQNDGMLYWVSNSVVYLTQVTHGISFVDVTVHDNNVYLLSTQNRIYRCSIGTGGAVVGSACTQITLTGSTEFDQLITVPSDFRGFAVSSCGIFIAPTSDLYWFSLSGVFITKSAGVTFVDIKLTSSSDTANTGTSVFMAASTSAVYAVTASSATISYALVSGKETKSCNPALNNVDSDTSPTFCGIARIYPLNTDMVYMTTGVASVVRAIIVSNTTISDTITRTPFPVYFLDNSSIIPLILDGMNYELVGNSNIPFPYVAINHSTPEVDDSTWDTTFSVDVSNRFFSTVSSAAVVSTPFMGSLHGLQAYYNRTNQILFGDPNVLPMCNLTKMQRIERAVAADARAALQYPYIYTSKAQNFTVNAHPNLTLLKLLMPYPFGEILNESGFFENTTTPAVLANVHFNTTMLAAVRNAYTPDFVYDCIFAGNAFPFQILTAAQQQLVRWIIYTAIQEQLAKCAENSPSYTGSDSSSSDSHDDMVPGCVPRVGIGNLTELVMPGMPYSNYNITVFIPEGLHYNFSISRCLDGTDWTNVTDYLQHATTPRTRKCGTGCIVSIAVVSAVVAAILVVAIVIATSKRRRLATVVAPAFTVEPKFASTLDMSSEEGSRNPLNR

SEQ ID NO: 61 - protein, 532 aa, Trypanosoma vivax
MRCHEPPTPPQLSATCCVAEEIDTYNKHLDALMQIIGDAIKNISTNEDNARARAEGLKGCNLHYVQFAVAHTEGSVVAARREAVKAQNTIKGSTSLLKKVTIDISNSFRNISSKCNELREKYPSLIPADKNSPPNITFKKAVQLYVKNFSTCNVMYAKKLLRLVAQSEKIEAEVSRAVERTNASTMELAKLDKVAVQLNKDITSNRTWAGCKLAEYHGQMNFVFMGFYVLLSDILDELHSLLKKSKSMQPTRLTQEEVRRALSKAEQVCHDVSRFVKSLGSTLRDFTNFVHRLRKEYLHGILRNASGFRESFERCYKVATNNSVTRLESTVEEITANNENIAAWESMTVHQWKDVSKKLRQSLLTVLGGSNEYILLYGYFQEFDSMSVREFSNTVRAFRQSITEMSVARNVVGVAAKTVAADRKRILCRSVLMFNKGTAGSESARKLYELCKTRMPVEEPDSSREDGVVGTSGSEEEISGKDGGTSFSVSDADYWEWDVPPKVLEESSGDLLYDTAVDLHTKRKSPFYQVGS

SEQ ID NO: 62 - protein, 607 aa, Trypanosoma vivax
MEVMLFDYFHVLPISCKPRNFCIAFMLMFLRFCPVFAMRCHEPPTPPQLSATCCVAEEIDTYNKHLDALMQIIGDAIKNISTNEDNARARAEGLKGCNLHYVQFAVAHTEGSVVAARREAVKAQNTIKGSTSLLKKVTIDISNSFRNISSKCNELREKYPSLIPADKNSPPNITFKKAVQLYVKNFSTCNVMYAKKLLRLVAQSEKIEAEVSRAVERTNASTMELAKLDKVAVQLNKDITSNRTWAGCKLAEYHGQMNFVFMGFYVLLSDILDELHSLLKKSKSMQPTRLTQEEVRRALSKAEQVCHDVSRFVKSLGSTLRDFTNFVHRLRKEYLHGILRNASGFRESFERCYKVATNNSVTRLESTVEEITANNENIAAWESMTVHQWKDVSKKLRQSLLTVLGGSNEYILLYGYFQEFDSMSVREFSNTVRAFRQSITEMSVARNVVGVAAKTVAADRKRILCRSVLMFNKGTAGSESARKLYELCKTRMPVEEPDSSREDGVVGTSGSEEEISGKDGGTSFSVSDADYWEWDVPPKVLEESSGDLLYDTAVDLHTKRKSPFYQVGSIAFGVFLLVVSCGVGILMFVRRWYAACVARSADGGTDC